(19) United States
(12) Patent Application Publication
(10) Pub. No.: US 2014/0081017 A1
(43) Pub. Date: Mar. 20, 2014

(54) Histone Deacetylase Inhibitors for Enhancing Activity of Antifungal Agents

(71) Applicant: METHYLGENE INC., (US)

(72) Inventors: Franck Raeppel, Montreal (CA); Stephane Raeppel, St-Lazare (CA); Arkadii Vaisburg, Kirkland (CA)

(73) Assignee: METHYLGENE INC., Montreal (CA)

(21) Appl. No.: 13/827,708

(22) Filed: Mar. 14, 2013

Related U.S. Application Data
(60) Provisional application No. 61/701,337, filed on Sep. 14, 2012.

(51) Int. Cl. C07C 259/06; C07C 311/46; C07D 295/088; C07D 233/61; C07D 295/13; C07D 211/62; C07D 295/15; C07D 333/24; C07C 323/41; C07C 271/28; C07D 209/08; C07D 273/01

(52) U.S. Cl. CPC C07C 259/06; C07D 209/08; C07C 311/46; C07D 295/088; C07D 233/61; C07D 273/01; C07D 211/62; C07D 295/15; C07D 333/24; C07C 323/41; C07C 271/28; C07D 295/13
USPC 540/467; 562/621; 548/469; 544/168; 548/575; 544/370; 548/338.1; 546/245; 544/165; 544/159; 549/77; 562/442; 544/391; 560/27; 544/400

(57) ABSTRACT

The present invention relates to compositions and methods to selectively treat fungal infection. More particularly, the invention relates to compounds, compositions thereof, and methods for selectively enhancing fungal sensitivity to antifungal compounds. The compositions of the invention are comprised of a combination of a histone deacetylase inhibitor, or an N-oxide, hydrate, solvate, pharmaceutically acceptable salt, agricultural formulation, prodrug or complex thereof, and an antifungal agent, the histone deacetylase inhibitor being a compound of Formula (I):

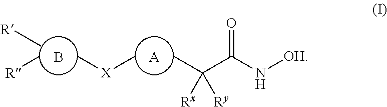

### BACKGROUND OF THE INVENTION

1. Field of the Invention

The invention relates to compounds, compositions thereof, and methods to treat fungal infection. More particularly, the invention relates to compounds, compositions thereof, and methods for enhancing fungal sensitivity to antifungal compounds.

2. Summary of the Related Art

In eukaryotic cells, nuclear DNA associates with histones to form a compact complex called chromatin. The histones constitute a family of basic proteins which are generally highly conserved across eukaryotic species. The core histones, termed H2A, H2B, H3, and H4, associate to form a protein core. DNA winds around this protein core, with the basic amino acids of the histones interacting with the negatively charged phosphate groups of the DNA. Approximately 146 base pairs of DNA wrap around a histone core to make up a nucleosome particle, the repeating structural motif of chromatin.

Csordas, (1990, Biochem. J., 286: 23-38) teaches that histones are subject to post-translational acetylation of amino groups of N-terminal lysine residues, a reaction that is catalyzed by histone acetyl transferase (HAT1). Acetylation neutralizes the positive charge of the lysine side chain, and is thought to impact chromatin structure. Indeed, Taunton et al. (1996, Science, 272: 408-411), teaches that access of transcription factors to chromatin templates is enhanced by histone hyperacetylation. Taunton et al. (supra) further teaches that an enrichment in under-acetylated histone H4 has been found in transcriptionally silent regions of the genome.

Histone acetylation is a reversible modification, with deacetylation being catalyzed by a family of enzymes termed histone deacetylases (HDACs). The molecular cloning of gene sequences encoding proteins with HDAC activity has established the existence of a set of discrete HDAC enzyme isoforms. Based on phylogenetic analyses and sequence homology to yeast Rpd3 (reduced potassium dependency 3), Hda1 and Sir2 (silent information regulator 2), HDACs are grouped into different classes (Jang and Grégoire, 2005, Molecular and Cellular Biology, 25(8):2873-2884). In humans there are 18 known HDACs, which are divided into four classes: class I (HDAC1, -2, -3 and -8; homologous to Rpd3), class II (HDAC4, -5, -6, -7, -9 and -10; related to Hda1), class III (Sirt1, -2, -3, -4, -5, -6 and -7; similar to Sir2) and class IV (HDAC11). Class I, II and IV HDACs are zinc-dependent enzymes. Class III HDACs are NAD⁺ dependent deacetylases. In Saccharomyces cerevisiae there are 10 known HDACs, which are divided into three classes: class I (Rpd3, Hos1 and Hos2), class II (Hda1 and Hos3), and class III (Sir2 and four Hst proteins, homologs of Sir2).

It has been unclear what roles these individual HDAC enzymes play. Trojer et al. (2003, Nucleic Acids Research, 31(14):3971-3981) indicate that HdaA and RpdA are major contributors to total HDAC activity of the filamentous fungus Aspergillusnidulans, with HdaA accounting for the main part of the HDAC activity.

Studies utilizing known HDAC inhibitors have established a link between acetylation and gene expression. Numerous studies have examined the relationship between HDAC and gene expression. Taunton et al., Science 272:408-411 (1996), discloses a human HDAC that is related to a yeast transcriptional regulator. Cress et al., J. Cell. Phys. 184:1-16 (2000), discloses that, in the context of human cancer, the role of HDAC is as a corepressor of transcription. Ng et al., TIBS 25: March (2000), discloses HDAC as a pervasive feature of transcriptional repressor systems. Magnaghi-Jaulin et al., Prog. Cell Cycle Res. 4:41-47 (2000), discloses HDAC as a transcriptional co-regulator important for cell cycle progression.

Numerous reports have been made describing inhibitors of HDAC activity. For example, Richon et al., Proc. Natl. Acad. Sci. USA, 95: 3003-3007 (1998), discloses that HDAC activity is inhibited by trichostatin A (TSA), a natural product isolated from Streptomyceshygroscopicus, which has been shown to inhibit histone deacetylase activity and arrest cell cycle progression in cells in the G1 and G2 phases (Yoshida et al., 1990, J. Biol. Chem. 265: 17174-17179; Yoshida et al., 1988, Exp. Cell Res. 177: 122-131), and by a synthetic compound, suberoylanilide hydroxamic acid (SAHA). Yoshida and Beppu (1988, Exper. Cell Res., 177: 122-131) teach that TSA causes arrest of rat fibroblasts at the G₁ and G₂ phases of the cell cycle, implicating HDAC in cell cycle regulation. Indeed, Finnin et al. (1999, Nature, 401:188-193), teach that TSA and SAHA inhibit cell growth, induce terminal differentiation, and prevent the formation of tumors in mice. Other non-limiting examples of compounds that serve as HDAC inhibitors include those of WO 01/38322 and WO 01/70675. The A. nidulans Hda1 enzyme is highly sensitive to the HDAC inhibitor TSA, while HosB has been shown to be highly resistant to both TSA and another HDAC inhibitor, HC toxin (Trojer et al., supra).

Smith and Edlind (2002, Antimicrobial Agents and Chemotherapy, 46(11):3532-3539) tested the ability of known HDAC pan-inhibitors TSA, apicidin, sodium butyrate and trapoxin to enhance the sensitivity of selected fungal species to azole antifungal agents. They found that only TSA was able to enhance the sensitivity of Candida albicans. However, the concentrations of TSA required were higher than those toxic to mammalian cells. TSA was not found to enhance the sensitivity of Candida glabrata.

The use of, and need for, antifungal agents is widespread and ranges from the treatment of mycotic infections in animals; to disinfectant formulations; to pharmaceuticals for human use. A major problem with current antifungal formulations is their toxicity to the infected host. This is particularly important in cases where many fungal infestations are opportunistic infections secondary to debilitating diseases, such as AIDS or from cancer chemotherapy or organ transplants. Correspondingly, at least for antifungal agents that are to be administered to humans and other animals, the therapeutic index is preferably such that toxicity is selective to the targeted fungus without being toxic to the host.

Serious fungal infections, caused mostly by opportunistic species such as Candida spp. and Aspergillus spp., are increasingly common in immunocompromised and other vulnerable patients (Georgopapadakou, 1998). They are important causes of morbidity and mortality in hospitalized patients and in HIV, cancer and transplant patients.

Infections by Candida are commonly treated with antifungal azoles which target lanosterol demethylase, an essential enzyme in ergosterol synthesis, the major component of the fungal membrane. Azoles are fungistatic and their use may be eroded by the emergence of azole-resistance, particularly in non-albicans Candida species such as Candida glabrata (Kaur et al., 2004). Further, azole treatment results in “trailing growth”, with surviving fungal cells becoming reservoirs for relapse. The major limitation of antifungal azoles is their general lack of fungicidal activity, which may contribute to treatment failures common with severely compromised patients.

Aspergillus fumigatus is the major Aspergillus species causing invasive aspergillosis (IA), a life-threatening disease with a mortality rate of 60-90%, whose incidence has increased dramatically in the past 20 years due to the increasing numbers of immunocompromised patients (Takaia et al., 2005). Current antifungal agents are limited in the treatment of IA by their poor in vivo efficacy and host toxicity (Latge 1999).

Drawbacks to current antifungal agents, such as the azoles, include development of resistance, possible drug-drug interactions and possible toxic liver effects.

An important factor in the resistance to azoles is thought to be the up-regulation of ERG genes that encode enzymes of the ergosterol biosynthetic pathway. Henry et al. demonstrated that exposure to azoles leads to the up-regulation of ERG11, the gene that encodes lanosterol demethylase, in Candida species. In the same study, up-regulation was also seen to occur in the five other ERG genes examined. Similar results were obtained with terbinafine and fenpropimorph, antifungals that act on other steps of the ergosterol pathway (Henry et al., 2000, Antimicrob. Agents Chemother. 44:2693-2700; Song et al., 2004 Antimicrob. Agents Chemother. 48(4):1136-1144).

### BRIEF SUMMARY OF THE INVENTION

It has been surprisingly found that certain inhibitors of histone deacetylase, particularly hydroxamate-based inhibitors of histone deacetylase, show synergistic activity with antifungal agents against fungal species, at concentrations of inhibitor not toxic to mammalian cells.

The present invention provides compounds, compositions thereof, and methods to selectively treat fungal infection. The present invention further provides compounds, compositions thereof, and methods for selectively enhancing fungal sensitivity to antifungal compounds. The compounds are hydroxamate-based inhibitors of histone deacetylase. The compounds of the invention are generally believed to be more active against a fungal histone deacetylase than a plant or mammalian histone deacetylase, and, generally, the inhibitory activity is believed to be specific for fungal histone deacetylase.

In a first aspect, the invention provides compounds for the selective treatment of fungal infection and enhancement of fungal sensitivity to antifungal compounds. The compounds are hydroxamate-based inhibitors of HDAC as well as N-oxides, hydrates, solvates, pharmaceutically acceptable salts, agricultural formulations, prodrugs, and complexes thereof.

In one embodiment of the first aspect, the histone deacetylase inhibitor is a compound of Formula (I):

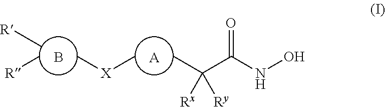

or an N-oxide, hydrate, solvate, pharmaceutically acceptable salt, agricultural formulation, prodrug or complex thereof, wherein groups A, B, R′, R″, X, R^{x} and R^{y} are defined herein.

In a second embodiment of the first aspect, the compound of Formula (I) is according to Formula (II)

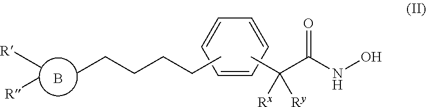

or an N-oxide hydrate, solvate, pharmaceutically acceptable salt, agricultural formulation, prodrug or complex thereof, wherein groups B, R′, R″, R^{x} and R^{y} are defined herein.

In a second aspect, the invention provides compositions comprising a histone deacetylase inhibitor and an antifungal agent for the selective enhancement of fungal sensitivity to antifungal agent. In one embodiment of the second aspect, the histone deacetylase inhibitor is a compound of Formula (I) or (II), and the antifungal agent is an azole.

In further aspects, the invention provides methods comprising contacting a fungal cell with a compound of the first aspect or a composition of the second aspect for (a) selectively sensitizing a fungal cell to an antifungal agent, (b) selectively enhancing the activity of an antifungal agent against a fungal cell, (c) selectively inhibiting fungal growth, (d) selectively treating a fungal infection, (e) selectively reducing resistance of a fungal cell to an antifungal agent, (f) selectively reducing antifungal agent-dependent upregulation of a gene in a fungal cell, (g) selectively inhibiting development of an antifungal agent-resistant fungal cell upon contacting the fungal cell with an antifungal agent, (h) selectively inhibiting expression of a gene involved in ergosterol biosynthesis or a gene encoding a multidrug transporter in a fungal cell during treatment of the fungal cell with an antifungal agent, (i) selectively promoting cidal effect of an antifungal agent on a fungal cell, or (j) selectively increasing the post-antibiotic effect of an antifungal agent on a fungal cell.

The foregoing merely summarizes certain aspects of the invention and is not intended to be limiting in nature. These aspects and other aspects and embodiments are described more fully below.

### DETAILED DESCRIPTION OF THE INVENTION

The present invention provides compounds, compositions thereof, and methods to selectively treat fungal infection. More particularly, this invention provides compounds, compositions thereof, and methods for selectively enhancing fungal sensitivity to antifungal compounds.

The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

### DEFINITIONS

For the purpose of the present invention, the following terms are defined below.

A large number of active antifungal agents have an azole functionality as part of their structure; such an antifungal agent is generally referred to as an “antifungal azole”, an “azole antifungal agent” or an “azole”.

The terms “selective”, “selectively” and “selectivity”, as used throughout herein, are intended to mean that the histone deacetylase inhibitory compounds and their use in the compositions and methods described herein achieve their purpose without being used in concentrations that are toxic to the host cells. “Host cells” are the cells of the animal or plant to be treated. Such selectivity is provided for the first time by the histone deacetylase inhibitory compounds according to the invention, and their use in the compositions and methods according to the invention.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-heteroaryl includes, for example, pyridyl and pyrimidyl.

The term “alkyl” is intended to mean a straight or branched chain aliphatic group having from 1 to 12 carbon atoms, preferably 1-8 carbon atoms, and more preferably 1-6 carbon atoms. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond.

The term “alkenyl” is intended to mean an unsaturated straight or branched chain aliphatic group with one or more carbon-carbon double bonds, having from 2 to 12 carbon atoms, preferably 2-8 carbon atoms, and more preferably 2-6 carbon atoms. Preferred alkenyl groups include, without limitation, ethenyl, propenyl, butenyl, pentenyl, and hexenyl.

The term “alkynyl” is intended to mean an unsaturated straight or branched chain aliphatic group with one or more carbon-carbon triple bonds, having from 2 to 12 carbon atoms, preferably 2-8 carbon atoms, and more preferably 2-6 carbon atoms. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “cycloalkyl” is intended to mean a saturated or unsaturated mono-, bi, tri- or poly-cyclic hydrocarbon group having about 3 to 15 carbons, preferably having 3 to 12 carbons, preferably 3 to 8 carbons, and more preferably 3 to 6 carbons. In certain preferred embodiments, the cycloalkyl group is fused to an aryl, heteroaryl or heterocyclic group. Preferred cycloalkyl groups include, without limitation, cyclopenten-2-enone, cyclopenten-2-enol, cyclohex-2-enone, cyclohex-2-enol, cyclopropyl, cyclobutyl, cyclopentyl, cyclopentenyl, cyclohexyl, cyclohexenyl, cycloheptyl, and cyclooctyl.

The terms “heterocyclyl”, “heterocyclic” or “heterocycle” are intended to mean a group which is a mono-, bi-, or polycyclic structure having from about 3 to about 20 atoms, wherein one or more atoms are independently selected from the group consisting of N, O, and S. The ring structure may be saturated, unsaturated or partially unsaturated. In certain preferred embodiments, the heterocyclic group is non-aromatic. In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” is intended to mean a mono-, bi-, tri- or polycyclic group having 5 to 14 ring atoms, preferably 5, 6, 9, or 10 ring atoms; having 6, 10, or 14 pi electrons shared in a cyclic array; and having, in addition to carbon atoms, between one or more heteroatoms independently selected from the group consisting of N, O, and S. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The term “aryl” is intended to mean a mono-, bi-, tri- or polycyclic C₆-C₁₄ aromatic moiety, preferably comprising one to three aromatic rings. Preferably, the aryl group is a C₆-C₁₀ aryl group, more preferably a C₆ aryl group. Preferred aryl groups include, without limitation, phenyl, naphthyl, anthracenyl, and fluorenyl.

Preferred heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thianthrenyl, thiazolyl, thienyl, thienothiazolyl, thienooxazolyl, thienoimidazolyl, thiophenyl, triazinyl, triazolyl (e.g., 1,2,3-triazolyl, 1,2,4-triazolyl, 1,2,5-triazolyl, 1,3,4-triazolyl), and xanthenyl.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, C₅-C₁₅ heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³)ₛ—NR³⁰R³¹, wherein s is from 0 (in which case the nitrogen is directly bonded to the moiety that is substituted) to 6, R³² and R³³ are each independently hydrogen, halo, hydroxyl or C₁-C₄alkyl, and R³⁰ and R³¹ are each independently hydrogen, cyano, oxo, hydroxyl, —C₁-C₈ alkyl, C₁-C₈ heteroalkyl, C₁-C₈ alkenyl, carboxamido, C₁-C₃ alkyl-carboxamido, carboxamido-C₁-C₃ alkyl, amidino, C₂-C₈hydroxyalkyl, C₁-C₃ alkylaryl, aryl-C₁-C₃ alkyl, C₁-C₃ alkylheteroaryl, heteroaryl-C₁-C₃ alkyl, C₁-C₃ alkylheterocyclyl, heterocyclyl-C₁-C₃ alkyl C₁-C₃ alkylcycloalkyl, cycloalkyl-C₁-C₃ alkyl, C₂-C₈ alkoxy, C₂-C₈ alkoxy-C₁-C₄alkyl, C₁-C₈ alkoxycarbonyl, aryloxycarbonyl, aryl-C₁-C₃ alkoxycarbonyl, heteroaryloxycarbonyl, heteroaryl-C₁-C₃ alkoxycarbonyl, C₁-C₈ acyl, C₀-C₈ alkyl-carbonyl, aryl-C₀-C₈ alkyl-carbonyl, heteroaryl-C₀-C₈ alkyl-carbonyl, cycloalkyl-C₀-C₈ alkyl-carbonyl, C₀-C₈ alkyl-NH-carbonyl, aryl-C₀-C₈ alkyl-NH-carbonyl, heteroaryl-C₀-C₈ alkyl-NH-carbonyl, cycloalkyl-C₀-C₈ alkyl-NH-carbonyl, C₀-C₈ alkyl-O-carbonyl, aryl-C₀-C₈ alkyl-O-carbonyl, heteroaryl-C₀-C₈ alkyl-O-carbonyl, cycloalkyl-C₀-C₈ alkyl-O-carbonyl, C₁-C₈ alkylsulfonyl, arylalkylsulfonyl, arylsulfonyl, heteroarylalkylsulfonyl, heteroarylsulfonyl, C₁-C₈ alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃ alkyl-, cycloalkyl-C₁-C₃ alkyl-, heterocyclyl-C₁-C₃ alkyl-, heteroaryl-C₁-C₃ alkyl-, or protecting group, wherein each of the foregoing is further optionally substituted with one more moieties listed in (a), above; or
R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹—), wherein said heterocyclyl may also be bridged (forming a bicyclic moiety with a methylene, ethylene or propylene bridge); wherein

X³⁰ is selected from the group consisting of C₁-C₈alkyl, C₂-C₈alkenyl-, C₂-C₈alkynyl-, —C₀-C₃alkyl —C₂-C₈alkenyl-C₀-C₃alkyl, C₀-C₃alkyl-C₂-C₈alkynyl-C₀-C₃alkyl, C₀-C₃ alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃ alkyl-, N(R³⁰)(R³¹)—C₀-C₃alkenyl-, N(R³⁰)(R³¹)—C₀-C₃alkynyl-, (N(R³⁰)(R³¹))₂—C═N—, C₀-C₃alkyl-S(O)₀₋₂—C₀-C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocycyl are optionally substituted with from 1 to 3 substituents from (a); and Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰), —N(R³⁰)—C(S)—N(R³¹)—, —N(R³⁰)—C(O)—O—, —O—C(O)—N(R³¹)—, —N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

When there are two optional substituents bonded to adjacent atoms of a ring structure, such as for example phenyl, thiophenyl, or pyridinyl, the substituents, together with the atoms to which they are bonded, optionally form a 5- or 6-membered cycloalkyl or heterocycle having 1, 2, or 3 annular heteroatoms.

In a preferred embodiment, a heterocyclic group is substituted on carbon, nitrogen and/or sulfur at one or more positions. Preferred substituents on nitrogen include, but are not limited to N-oxide, alkyl, aryl, aralkyl, alkylcarbonyl, alkylsulfonyl, arylcarbonyl, arylsulfonyl, alkoxycarbonyl, or aralkoxycarbonyl. Preferred substituents on sulfur include, but are not limited to, oxo and C₁₋₆alkyl.

In addition, substituents on cyclic moieties (i.e., cycloalkyl, heterocyclyl, aryl, heteroaryl) include 5-6 membered mono- and 9-14 membered bi-cyclic moieties fused to the parent cyclic moiety to form a bi- or tri-cyclic fused ring system. Substituents on cyclic moieties also include 5-6 membered mono- and 9-14 membered bi-cyclic moieties attached to the parent cyclic moiety by a covalent bond to form a bi- or tri-cyclic bi-ring system. For example, an optionally substituted phenyl includes, but is not limited to, the following:

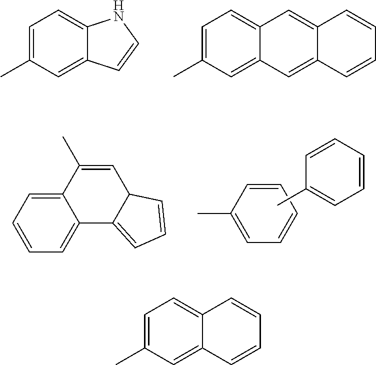

The term “polyether” is intended to mean a group comprising repeating ether units that terminate with an alkoxy group and has the general formula —O(CₓH₂ₓ)O)_{y}C_{z}H_{2z+1}, where x is 1-10, y is 1-20, and z is 1-6. The repeating units and terminating group can be optionally substituted by the replacement of any hydrogen with alkyl, alkoxy, aryl, heteroatom, alkylhalide or halogen as defined herein.

The term “pharmaceutically acceptable carrier” is intended to mean a non-toxic material that is compatible with a biological system in a cell, cell culture, tissue sample or body and that does not interfere with the effectiveness of the biological activity of the active ingredient(s). Thus, compositions according to the invention may contain, in addition to the inhibitor and antifungal agent, diluents, excipients, fillers, salts, buffers, stabilizers, solubilizers, and/or other materials well known in the art. Examples of the preparation of pharmaceutically acceptable formulations are described in, e.g., Remington's Pharmaceutical Sciences, 18th Edition, ed. A. Gennaro, Mack Publishing Co., Easton, Pa., 1990.

The active compounds of a composition of the invention are included in the pharmaceutically acceptable carrier in an amount sufficient to deliver an effective desired amount without causing serious toxic effects to an individual to which the composition is administered. The term “hydroxamate-based inhibitor of histone deacetylase” is intended to mean a compound which is an inhibitor of histone deacetylase and which includes a hydroxamate moiety.

It will be understood that the characteristics of the carrier, will depend on the route of administration for a particular application.

The term “pharmaceutically acceptable salt”, “salt”, or “salts” is intended to mean a salt that retains the desired biological activity of a compound of the present invention in an animal or plant and exhibits minimal or no undesired toxicological effects. Examples of such salts include, but are not limited to acid addition salts formed with inorganic acids, such as, for example, hydrochloric acid, hydrobromic acid, sulfuric acid, phosphoric acid, nitric acid, and the like, and salts formed with organic acids such as acetic acid, oxalic acid, tartaric acid, succinic acid, maleic acid, ascorbic acid, benzoic acid, tannic acid, pamoic acid, alginic acid, polyglutamic acid, naphthalenesulfonic acid, naphthalenedisulfonic acid, trifluoroacetic acid, toluenesulfonic acid, methanesulfonic acid, citric acid and polygalacturonic acid. The compounds can also be in the form of pharmaceutically acceptable quaternary salts known by those skilled in the art, which specifically include the quaternary ammonium salt of the formula —NR⁺+Z⁻, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). Non-toxic pharmaceutical base addition salts include salts of bases such as sodium, potassium, calcium, ammonium, and the like. Those skilled in the art will recognize a wide variety of non-toxic pharmaceutically acceptable addition salts.

The term “agricultural formulation” is intended to mean a composition comprising a compound of the invention optionally combined with an antifungal agent that is formulated with one or more additive in a manner to enhance the effectiveness, handling, stability, storage and application of the composition. The formulation can be in a solid form, such as granules, microgrannules or dust, or in powdered form which can be combined with water for spray application. Other agricultural formulations are solutions for low-volume spraying, fogging or fumigating. Commonly used additives, also referred to as adjuvants, include, but are not limited to surfacants, non-ionic surfacants, emulsifiers, wetting agents, diluents, and spreader-stickers.

The term “histone deacetylase inhibitor” is intended to mean a compound, which is capable of interacting with a histone deacetylase and inhibiting the activity of the histone deacetylase. In some preferred embodiments, such reduction of activity is at least about 50%, more preferably at least about 75%, and more preferably at least about 90%, and still more preferably at least about 95%. In some preferred embodiments of the invention the compound is a compound having a structure as defined herein.

The term “antifungal agent” or “fungicide” is intended to mean a substance capable of inhibiting or preventing the growth, viability and/or reproduction of a fungal cell. Antifungal agents are capable of preventing or treating a fungal infection in an animal or plant. An antifungal agent may be a broad spectrum antifungal agent, but can also be specific to one or more particular species of fungus.

Antifungal agents are commonly ergosterol synthesis inhibitors, and include, but are not limited to azoles, allylamines and morpholines. Antifungal agents are also substances with alternative or unknown mechanisms of action, such as, for example, echinocandins, amphotericin B, ciclopirox, chlorophetanol, chlorphensin, filipin, flucytosine, griseofulvin, haloprogin, hamycin, natamycin, nikkomycins, preferably nikkomycin Z, nystatin, pimaricin, polygodial, sulbentine, taurolidine, ticlatone, tolciclate, tolnaftate and undecylenic acid. Echinocandins include, but are not limited to anidulafungin, caspofungin and micafungin. Azole antifungal agents include imidazoles, triazoles and thiazoles. Imidazole antifungal agents include, but are not limited to binonazole, butoconazole, clomidazole, clotrimazole, croconazole, econazole, fenticonazole, isoconazole, ketoconazole, miconazole, neticonzaole, omoconazole, oxiconazole, sertazonazole, sulconazole, and tioconazole. Triazole antifungal agents include, but are not limited toalbaconazole, fluconazole, fosfluconaole, hexaconazole, isavuconazole, itraconazole, posaconazole, ravuconazole, terconazole and voriconazole. Thiazole antifungal agents include, but are not limited to abafungin and dimazole. Like azoles, fenpropimorph is an ergosterol synthesis inhibitor, but acts on the ergosterol reductase (ERG₂₄) step of the synthesis pathway. Terbinafine, is also an ergosterol inhibitor, but acts on the squalene eposidase (ERG₁) step.

The terms “histone deacetylase inhibitor” and “inhibitor of histone deacetylase” are intended to mean a compound which is capable of interacting with a histone deacetylase and inhibiting its enzymatic activity. “Inhibiting histone deacetylase enzymatic activity” means reducing the ability of a histone deacetylase to remove an acetyl group from a histone. In some preferred embodiments, such reduction of histone deacetylase activity is at least about 50%, more preferably at least about 75%, and still more preferably at least about 90%. In other preferred embodiments, histone deacetylase activity is reduced by at least 95% and more preferably by at least 99%.

The histone deacetylase inhibitor may be any molecule that effects a reduction in the activity of a histone deacetylase. This includes proteins, peptides, DNA molecules (including antisense), RNA molecules (including RNAi and antisense) and small molecules.

Preferably, such inhibition is specific, i.e., the histone deacetylase inhibitor reduces the ability of a histone deacetylase to remove an acetyl group from a histone at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

The term “effective amount” as employed herein is an amount of a compound of the invention that achieves the effect which is intended with its application. The amount of a compound of the invention which constitutes an “effective amount” will vary depending on the compound, the intended use, the disease state and its severity, the age of the patient to be treated, and the like. The effective amount can be determined routinely by one of ordinary skill in the art.

The term “patient” as employed herein for the purposes of the present invention includes humans and other animals, particularly mammals, and other organisms. Thus, the compounds, compositions and methods of the present invention are applicable to both human therapy and veterinary applications. In a preferred embodiment the patient is a mammal, and in a most preferred embodiment the patient is human.

The terms “treating” or “treatment” as used herein covers the treatment of a disease-state in an animal or plant, which disease-state is characterized by pathogen invasion and includes at least one of: (i) preventing the disease-state from occurring in an animal or plant, in particular, when such animal or plant is predisposed to the disease-state but has not yet been diagnosed as having it; (ii) inhibiting the disease-state, i.e., arresting its development; and (iii) relieving the disease-state, i.e., causing regression of the disease-state. In a preferred embodiment of the present invention the animal is a mammal, more preferably a human. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

The present invention also includes prodrugs of compounds of the invention. The term “prodrug” is intended to represent covalently bonded carriers, which are capable of releasing the active ingredient when the prodrug is administered to a mammalian subject, or to a fungal cell. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the invention include compounds wherein an amino, hydroxy, carboxylic or a similar group is modified. Examples of prodrugs include, but are not limited to esters (e.g., acetate, formate, and benzoate derivatives), carbamates (e.g., N,N-dimethylaminocarbonyl) of hydroxy or amino functional groups), amides (e.g., trifluoroacetylamino, acetylamino, and the like), and the like.

The compounds of the invention may be administered, for example, as is or as a prodrug, for example in the form of an in vivo hydrolyzable ester or in vivo hydrolyzable amide. An in vivo hydrolyzable ester of a compound of the invention containing a carboxy or hydroxy group is, for example, a pharmaceutically acceptable ester which is hydrolyzed in the organism being treated, preferably a human or animal body, to produce the parent acid or alcohol. Alternatively, hydrolysis occurs in a fungal cell. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈-cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₆-alkoxycarbonyloxyethyl esters (e.g., 1-methoxycarbonyloxyethyl) and may be formed at any appropriate carboxy group in the compounds of this invention.

An in vivo hydrolyzable ester of a compound of the invention containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an invivo hydrolyzable amide of a compound of the invention containing a carboxy group is, for example, a N—C₁₋₆-alkyl or N,N-di-C₁₋₆-alkyl amide such as N-methyl, N-ethyl, N-propyl, N,N-dimethyl, N-ethyl-N-methyl or N,N-diethyl amide.

The present invention is in no way intended to be limited to purely human applications and is intended to encompass for example veterinary, agricultural and aquatic applications, including for example methods for treating fungal infections of non-human mammals, fish and plants. Smith and Edlind (supra) for example showed that TSA reduced the minimum inhibitory concentration of the morpholine fenpropimorph, an agricultural fungicide whose enzyme targets in the ergosterol biosynthetic pathway follow those of allylamines and azoles.

### Compounds

In a first aspect, the invention provides compounds for the selective treatment of fungal infection and enhancement of fungal sensitivity to antifungal compounds. The compounds are hydroxamate-based inhibitors of HDAC, as well as N-oxides, hydrates, solvates, pharmaceutically acceptable salts, agricultural formulations, prodrugs and complexes thereof.

In one embodiment of the first aspect, the histone deacetylase inhibitor is a compound of Formula (I):

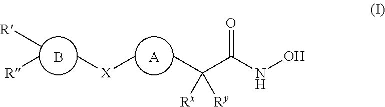

or an N-oxide, hydrate, solvate, pharmaceutically acceptable salt, agricultural formulation, prodrug or complex thereof, wherein

A is aryl, cycloalkyl, heterocycloalkyl, or heteroaryl, each of which is optionally substituted with alkyl, alkoxy, haloalkyl or halogen;

B is aryl, cycloalkyl, heterocycloalkyl, or heteroaryl, each of which is optionally substituted with alkyl, alkoxy or halogen;

R′ and R″ are each independently H, alkoxy, hydroxyl, alkyl, amino, halogen, carboxylic, N-hydroxyacetamide, phenyl, polyether, —C(O)NR¹R², —O-alkyl-NR¹R², —NHC(O)R³, —SO₂NHCH₂CH₂R³—NHC(O)NHCH₂CH₂R⁴, —NHSO₂CH₂CH₂R⁴ or CH₂C(O)NHOH where

R¹ and R² are each independently hydrogen, alkyl, thioalkyl, polyether, or combined with the nitrogen to which they are attached to form a heterocyclic ring, each of which are optionally substituted with aminoalkyl, thioalkyl, aryl, alkenyl heterocyclic, heteroaryl;

R³ is hydrogen, alkyl, thioalkyl, alkoxy, hydroxyalkyl, heterocyclic, or polyether, each of which are optionally substituted;

R⁴ is aryl, cycloalkyl, heterocycloalkyl, heteroaryl, each of which are optionally substituted; or

R′ and R″ occur on adjacent carbon atoms and combine to form a fused 1-methyl-2,3-dihydro-1H-pyrrole;

X is C₃-C₆ alkyl optionally and independently substituted at one or more positions with one or two alkyl, halo, or hydroxyl groups, or one oxo, amino, or imino group; and

R^{x} and R^{y} are each independently hydrogen or alkyl;

provided that when A is phenyl, X is unsubstituted butyl, and R^{x}, R^{Y}, R′ and R″ are H, B is not 1-H-indole; and

when A and B are phenyl and X is unsubstituted C₃-C₅ alkyl, at least one of R^{x}, R^{Y}, R′ or R″ is not H.

The invention further comprises subgenera of Formula (I) in which the substituents are selected as any and all combinations of one or more of structural Formula (I), A, B, R′, R″. R¹, R², R³, R⁴, R^{x}, R^{y} and X as defined herein, including without limitation, the following:

Structural Formula (I) is One of Formulae (1a)-(1v) where A is:

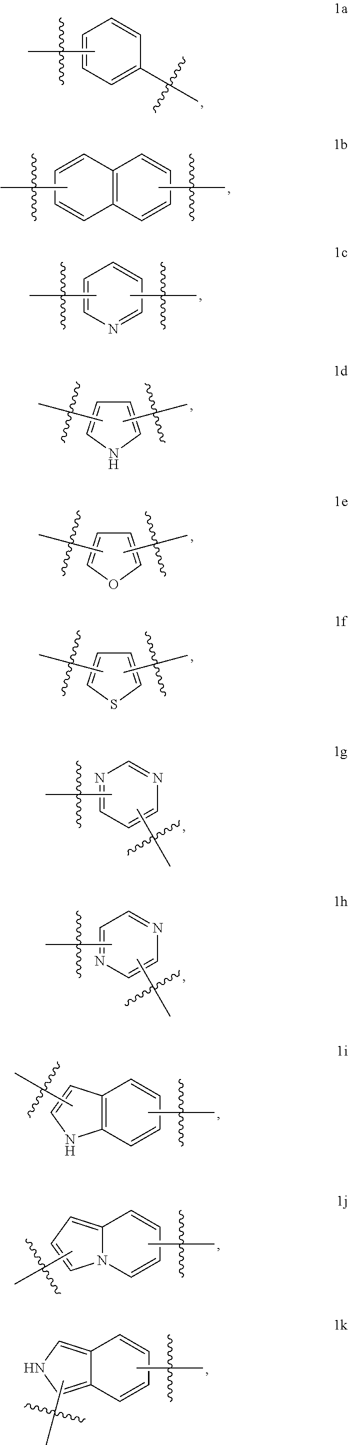

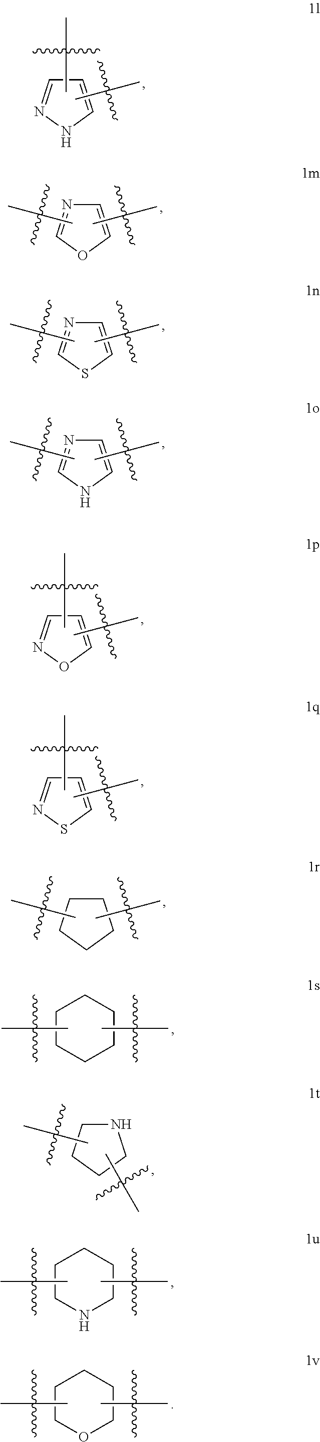

B in any of Formula (I) and (1a)-(1v) is Selected from One of the Following Groups (2a)-(2v):

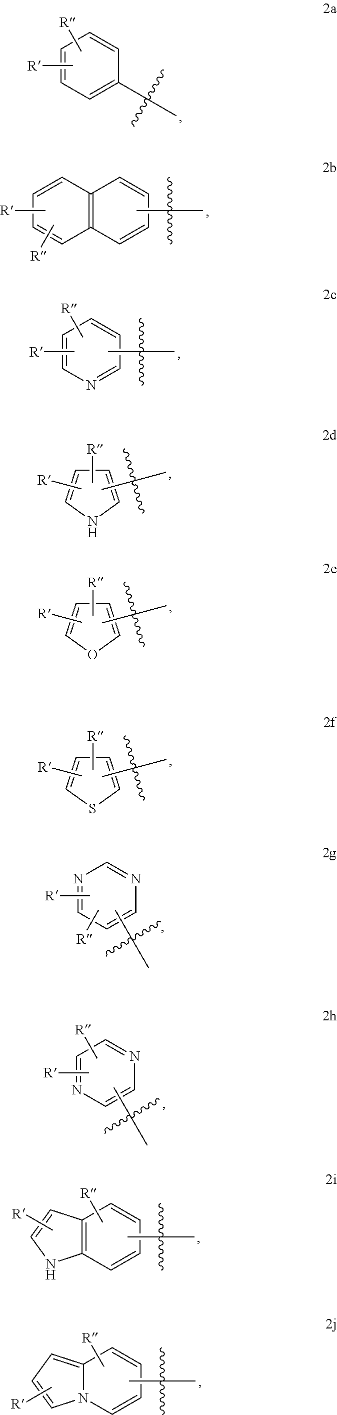

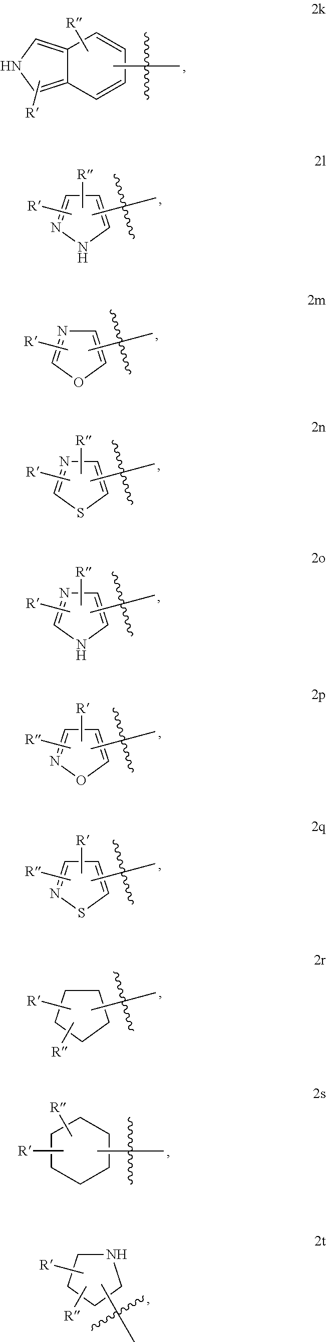

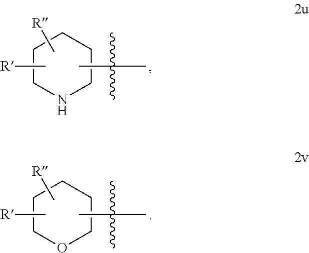

R′ and R″ in any of Formula (I) and (1a)-(1v) are Selected from One of the Following Groups (3a)-(3o):
(3a) R′ and R″ are each independently H, —C(O)NR¹R², —O-alkyl-NR¹R², —NHC(O)R³, —SO₂NHCH₂CH₂R³, —NHSO₂CH₂CH₂R⁴, —NHC(O)NHCH₂CH₂R⁴ or CH₂C(O)NHOH where R¹ and Rare defined according to groups (4a)-(4k), R³ is defined according to groups (5a)-(5e), and R⁴ is defined according to groups (6a)-(6f) below.
(3b) R′ and R″ are each independently H, —NHC(O)R³, —SO₂NHCH₂CH₂R³, —NHSO₂CH₂CH₂R⁴, —NHC(O)NHCH₂CH₂R⁴, or where R³ is defined according to groups (5a)-(5e), and R⁴ is defined according to groups (6a)-(6f) below.
(3c) R′ and R″ are each independently H, —C(O)NR¹R² or —O-alkyl-NR¹R², where R¹ and R² are defined according to groups (4a)-(4k) below.
(3d) R′ is H and R″ is —C(O)NR¹R², —O-alkyl-NR¹R², —NHC(O)R³, —SO₂NHCH₂CH₂R³, —NHSO₂CH₂CH₂R⁴, or —NHC(O)NHCH₂CH₂R⁴, where R′, R², R³ and R⁴ are as described below.
(3e) R′ is H and R″ is —NHC(O)R³, —NHC(O)NHCH₂CH₂R⁴, —SO₂NHCH₂CH₂R³, or —NHSO₂CH₂CH₂R⁴ where R³ is defined according to groups (5a)-(5e), and R⁴ is defined according to groups (6a)-(6f) below.
(3f) R′ is H and R″ is —C(O)NR¹R² or —O-alkyl-NR¹R², where R¹ and R² are defined according to groups (4a)-(4k) below.
(3g) R′ and R″ are each independently hydrogen, alkoxy, hydroxyl, alkyl, amino, halogen, carboxylic, N-hydroxyacetamide, phenyl, polyether, or R′ and R″ occur on adjacent carbon atoms and combine to form a fused 1-methyl-2,3-dihydro-1H-pyrrole.
(3h) R′ and R″ are each independently hydrogen, alkoxy, hydroxyl, alkyl, amino, halogen, carboxylic, N-hydroxyacetamide, phenyl, or

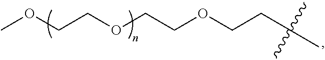

where n is 1-20.
(3i) R′ and R″ are each independently hydrogen, methoxy, ethoxy, hydroxyl, methyl, ethyl, fluoro, chloro, or bromo.
(3j) R′ and R″ occur on adjacent carbon atoms and combine to form a fused 1-methyl-2,3-dihydro-1H-pyrrole.
(3k) R′ is hydrogen and R″ is alkoxy, hydroxyl, alkyl, amino, halogen, carboxylic, N-hydroxyacetamide, phenyl, or polyether.
(3l) R′ is hydrogen and R″ is alkoxy, hydroxyl, alkyl, amino, halogen, carboxylic, N-hydroxyacetamide, phenyl, or

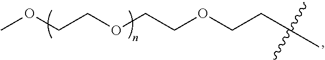

where n is 2-5.
(3m) R′ is hydrogen and R″ is methoxy, ethoxy, hydroxyl, methyl, ethyl, fluoro, chloro, or bromo.
(3n) R′ and R″ are both fluoro, hydroxyl, or methoxy.
(3o) R′ and R″ combine with group 2a, where B is substituted phenyl, to form:

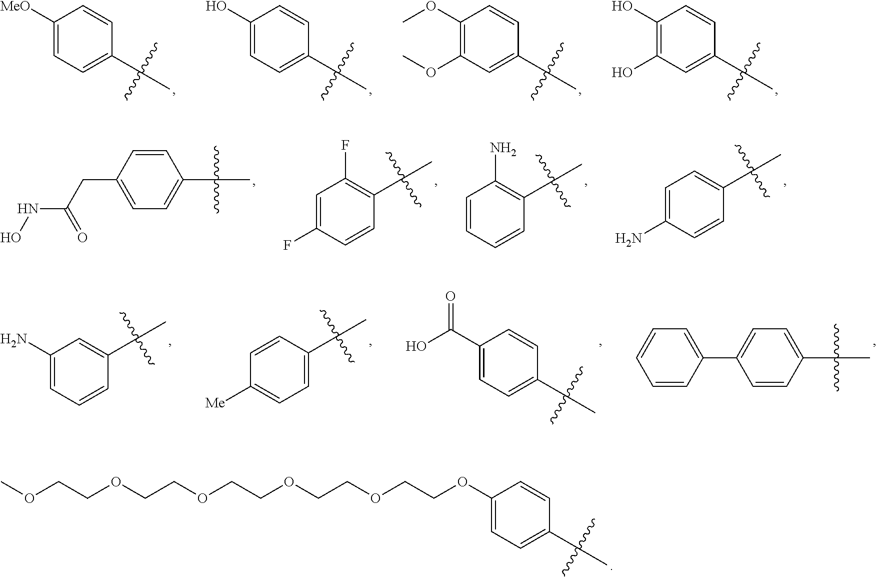

R¹ and R² in any of Formula (I) and (1a)-(1v) are Selected from One of the Following Groups (4a)-(4k):
(4a) R¹ and R² are each independently hydrogen, alkyl, thioalkyl, polyether, or combine with the nitrogen to which they are attached to form a heterocyclic ring, each of which are optionally substituted.
(4b) R¹ and R² are each independently hydrogen, alkyl, thioalkyl, polyether, or combine with the nitrogen to which they are attached to form morpholine, pyrrolidine, piperazine, piperidine,

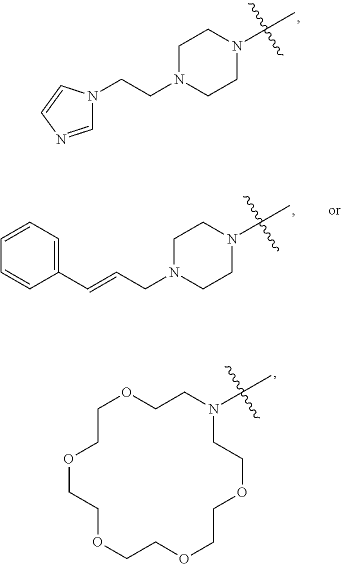 [SMILES: *N1CCN(CCn2ccnc2)CC1 ; *N1CCN(C/C=C/c2ccccc2)CC1 ; *N1CCOCCOCCOCCOCCOCC1]

each of which are optionally substituted.
(4c) R¹ and R² are each independently hydrogen, alkyl, thioalkyl, polyether,

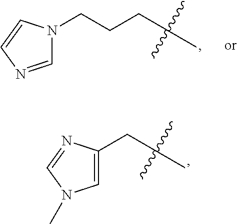

each of which are optionally substituted.
(4d) R¹ and R² are each independently hydrogen, alkyl, thioalkyl, or

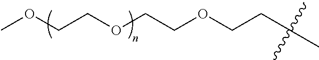

where n is 1-20 and each of which are optionally substituted.
(4e) R¹ and R² are each independently methyl, ethyl, or isopropyl.
(4f) R¹ and R² are each independently hydrogen,

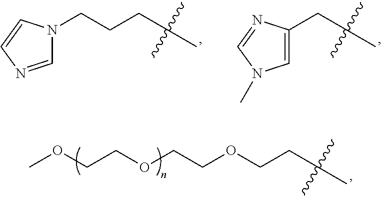

where n is 1-20, or combine with the nitrogen to which they are attached to form

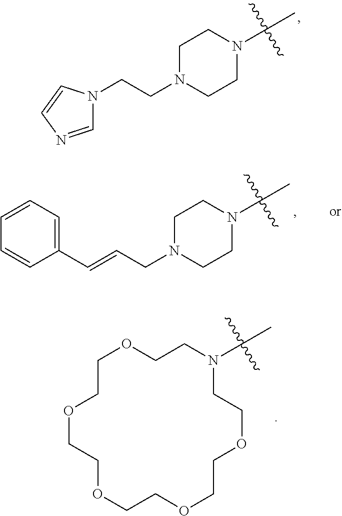 [SMILES: *N1CCN(CCn2ccnc2)CC1 ; *N1CCN(C/C=C/c2ccccc2)CC1 ; *N1CCOCCOCCOCCOCCOCC1]

(4g) R¹ is hydrogen and R² is alkyl, thioalkyl, polyether, or combine with the nitrogen to which they are attached to form a heterocyclic ring, each of which are optionally substituted with aminoalkyl, thioalkyl, aryl, alkenyl heterocyclic, heteroaryl.
(4h) R¹ is hydrogen and R² is alkyl, thioalkyl, polyether,

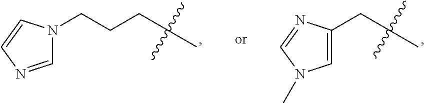

each of which are optionally substituted.
(4i) R¹ is hydrogen and R² is alkyl, thioalkyl, or

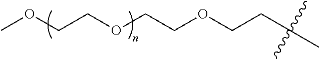

where n is 2-10 and each of which are optionally substituted.
(4j) R¹ is hydrogen and R² is methyl, ethyl, or isopropyl.
(4k) R¹ is hydrogen and R² is

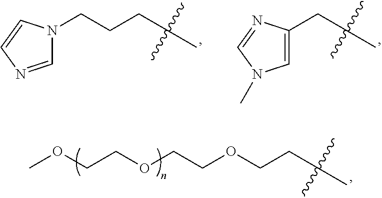

where n is 2-6.

R³ in any of Formula (I) and (1a)-(1v) is Selected from One of the Following Groups (5a)-(5e):
(5a) R³ is hydrogen, alkyl, thioalkyl, alkoxy, hydroxyalkyl, heterocyclic, or polyether, each of which are optionally substituted.
(5b) R³ is hydrogen, alkyl, thioalkyl, alkoxy, or hydroxyalkyl, each of which are optionally substituted, or

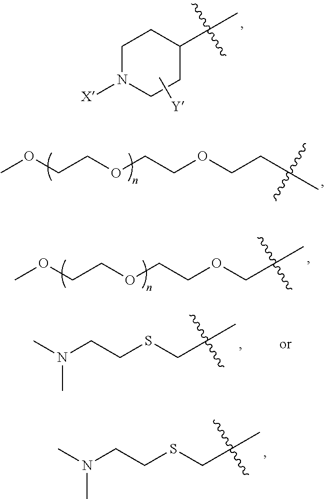

where n is 1-20, X′ is hydrogen, alkyl, hydroxyl, haloalkyl, phenyl or benzyl, and Y′ is hydrogen, alkyl, hydroxyl, haloalkyl or halogen.
(5c) R³ is hydrogen, methyl, ethyl, hydroxylmethyl, methoxy, ethoxy, or N-methyl piperdyl.
(5d) R³ is

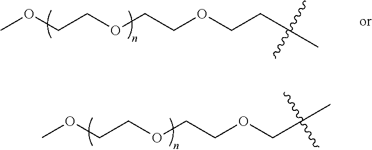

where n is 2-6.
(5e) R³ is methyl.

R⁴ in any of Formula (I) and (1a)-(1v) is Selected from One of the Following Groups (6a)-(6f):
(6a) R⁴ is aryl, cycloalkyl, heterocycloalkyl, heteroaryl, each of which are optionally substituted.
(6b) R⁴ is aryl, cycloalkyl, heterocycloalkyl, thiazolyl, ozazolyl, pyridyl, morpholine, pyrrolidine, piperazine, or piperidine, each of which is optionally substituted.
(6c) R⁴ is aryl, cycloalkyl,

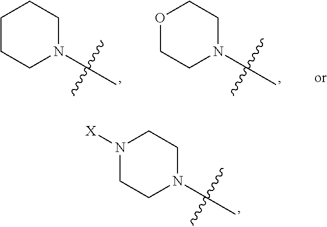

each of which are optionally substituted, and where X is hydrogen, alkyl, hydroxyl, haloalkyl, phenyl or benzyl.
(6d) R⁴ is aryl, cyclohexyl, or cyclopentyl, each of which is optionally substituted.
(6e) R⁴ is optionally substituted phenyl.
(6f) R⁴ is

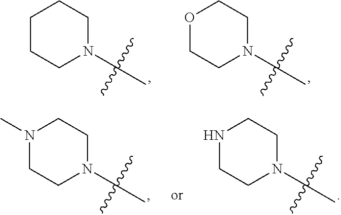

X in any of Formula (I) and (1a)-(1v) is Selected from One of the Following Groups (7a)-(7i):
(7a) X is C₃-C₆ alkyl, optionally and independently substituted at one or more positions with one or two alkyl, halo, or hydroxyl groups, or one oxo, amino, or imino group.
(7b) X is propyl, butyl, pentyl or hexyl, each independently substituted at one or more positions with one alkyl, halo, or hydroxyl groups, or one oxo, amino, or imino group.
(7c) X is propyl, butyl, pentyl or hexyl, each independently substituted at one or more positions with two methyl, ethyl, fluoro, chloro, bromo or hydroxyl groups, or one oxime group.
(7d) X is propyl, butyl, pentyl or hexyl, each independently substituted at one or more positions with one methyl, ethyl, fluoro, chloro, bromo or hydroxyl group.
(7e) X is propyl, butyl, pentyl or hexyl, each optionally and independently substituted at one or more positions with two methyl, ethyl, fluoro, chloro, bromo or hydroxyl groups.
(7f) X is butyl, independently substituted at one or more positions with one or two methyl, ethyl, fluoro, chloro, bromo or hydroxyl groups, or one oxime group.
(7g) X is butyl, independently substituted at one or more positions with one methyl, ethyl, fluoro, chloro, bromo or hydroxyl group.
(7h) X is butyl, optionally and independently substituted at one or more positions with two methyl, ethyl, fluoro, chloro, bromo or hydroxyl groups.
(7i) X is:

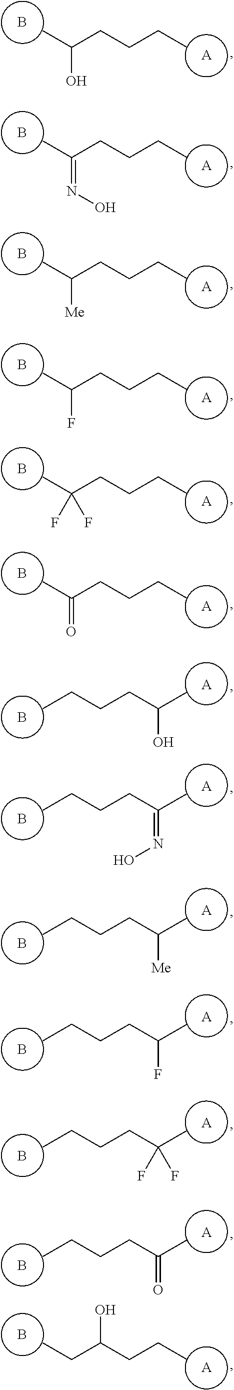

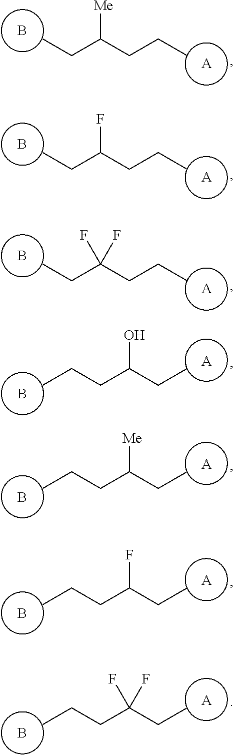

(7j) X is unsubstituted propyl.
(7k) X is unsubstituted butyl.
(7l) X is unsubstituted pentyl.
(7m) X is unsubstituted hexyl.

R^{x} and R^{y} in any of Formula (I) and (1a)-(1v) are Selected from One of the Following Groups (8a)-(8h):
(8a) R^{x} and R^{y} are each independently hydrogen or alkyl.
(8b) R^{x} is hydrogen and R^{y} is alkyl.
(8c) R^{x} and R^{y} are both hydrogen.
(8d) R^{x} and R^{y} are both alkyl.
(8e) R^{x} is hydrogen and R^{y} is methyl, ethyl, propyl, isopropyl, butyl, or tert-butyl.
(8f) R^{x} is hydrogen and R^{y} is methyl.
(8g) R^{x} and R^{y} are both methyl, ethyl, propyl, or butyl.
(8h) R^{x} and R^{y} are both methyl.

Particular embodiments of the first aspect of the invention include compounds of Formula (I) composed of any combination of chemical groups as defined. These compounds are represented by Formulae I-A-1-30 in Table 1, wherein each entry is a group number as defined above (e.g., (8c) refers to R^{x} and R^{y} both being hydrogen). A dash “-” indicates that the variable is as defined in Formula (I) or defined according to any one of the applicable variable definitions (1a)-(8h) [e.g., when A is a dash, it can be either as defined for Formula (I) or any one of definitions [(1a)-(1v)]. A “X” indicates that the group is not applicable to the formula (e.g., when R′/R″ is —C(O)NR¹R², R³ is not applicable).

TABLE 1
Embodiments of Formula (I).

| Formula | A | B | R′/R″ | R¹/R² | R³ | R⁴ | X | R^{x}/R^{y} |
|---|---|---|---|---|---|---|---|---|
| I-A-1 | 1c | 2c | 3a | 4f | 5b | 6d | 7e | 8b |
| I-A-2 | 1c | 2g | 3a | 4f | 5c | 6f | 7i | 8c |
| I-A-3 | 1c | 2s | 3i | — | — | — | 7i | 8h |
| I-A-4 | 1g | 2c | 3a | 4f | 5b | 6d | 7e | 8b |
| I-A-5 | 1g | 2g | 3a | 4f | 5c | 6f | 7i | 8c |
| I-A-6 | 1g | 2s | 3i | — | — | — | 7i | 8h |
| I-A-7 | 1m | 2c | 3a | 4f | 5b | 6d | 7e | 8b |
| I-A-8 | 1m | 2g | 3a | 4f | 5c | 6f | 7i | 8c |
| I-A-9 | 1m | 2s | 3i | — | — | — | 7i | 8h |
| I-A-10 | 1n | 2c | 3a | 4f | 5b | 6d | 7e | 8b |
| I-A-11 | 1n | 2g | 3a | 4f | 5c | 6f | 7i | 8c |
| I-A-12 | 1n | 2s | 3i | — | — | — | 7i | 8h |
| I-A-13 | 1q | 2c | 3a | 4f | 5b | 6d | 7e | 8b |
| I-A-14 | 1q | 2g | 3a | 4f | 5c | 6f | 7i | 8c |
| I-A-15 | 1q | 2s | 3i | — | — | — | 7i | 8h |
| I-A-16 | 1c | 2c | 3a | 4f | 5c | 6d | 7i | 8b |
| I-A-17 | 1c | 2s | 3a | 4f | 5b | 6f | 7e | 8h |
| I-A-18 | 1c | 2g | 3i | — | — | — | 7i | 8c |
| I-A-19 | 1g | 2c | 3a | 4f | 5c | 6d | 7i | 8b |
| I-A-20 | 1g | 2s | 3a | 4f | 5b | 6f | 7e | 8h |
| I-A-21 | 1g | 2g | 3i | — | — | — | 7i | 8c |
| I-A-22 | 1m | 2c | 3a | 4f | 5c | 6d | 7i | 8b |
| I-A-23 | 1m | 2g | 3a | 4f | 5b | 6f | 7e | 8h |
| I-A-24 | 1m | 2s | 3i | — | — | — | 7i | 8c |
| I-A-25 | 1n | 2c | 3a | 4f | 5c | 6d | 7i | 8b |
| I-A-26 | 1n | 2g | 3a | 4f | 5b | 6f | 7e | 8h |
| I-A-27 | 1n | 2s | 3i | — | — | — | 7i | 8c |
| I-A-28 | 1q | 2c | 3a | 4f | 5c | 6d | 7i | 8b |
| I-A-29 | 1q | 2g | 3a | 4f | 5b | 6f | 7e | 8h |
| I-A-30 | 1q | 2s | 3i | — | — | — | 7i | 8c |
| I-A-31 | 1c | 2c | 3a | 4f | 5b | 6d | 7k | 8b |
| I-A-32 | 1c | 2g | 3a | 4f | 5c | 6f | 7l | 8c |
| I-A-33 | 1c | 2s | 3i | — | — | — | 7m | 8h |
| I-A-34 | 1g | 2c | 3a | 4f | 5b | 6d | 7k | 8b |
| I-A-35 | 1g | 2g | 3a | 4f | 5c | 6f | 7l | 8c |
| I-A-36 | 1g | 2s | 3i | — | — | — | 7m | 8h |
| I-A-37 | 1m | 2c | 3a | 4f | 5b | 6d | 7k | 8b |
| I-A-38 | 1m | 2g | 3a | 4f | 5c | 6f | 7l | 8c |
| I-A-39 | 1m | 2s | 3i | — | — | — | 7m | 8h |
| I-A-40 | 1n | 2c | 3a | 4f | 5b | 6d | 7k | 8b |
| I-A-41 | 1n | 2g | 3a | 4f | 5c | 6f | 7l | 8c |
| I-A-42 | 1n | 2s | 3i | — | — | — | 7m | 8h |
| I-A-43 | 1q | 2c | 3a | 4f | 5b | 6d | 7k | 8b |
| I-A-44 | 1q | 2g | 3a | 4f | 5c | 6f | 7l | 8c |
| I-A-45 | 1q | 2s | 3i | — | — | — | 7m | 8h |
| I-A-46 | 1c | 2c | 3a | 4f | 5c | 6d | 7k | 8b |
| I-A-47 | 1c | 2s | 3a | 4f | 5b | 6f | 7l | 8h |
| I-A-48 | 1c | 2g | 3i | — | — | — | 7m | 8c |
| I-A-49 | 1g | 2c | 3a | 4f | 5c | 6d | 7k | 8b |
| I-A-50 | 1g | 2s | 3a | 4f | 5b | 6f | 7l | 8h |
| I-A-51 | 1g | 2g | 3i | — | — | — | 7m | 8c |
| I-A-52 | 1m | 2c | 3a | 4f | 5c | 6d | 7k | 8b |
| I-A-53 | 1m | 2g | 3a | 4f | 5b | 6f | 7l | 8h |
| I-A-54 | 1m | 2s | 3i | — | — | — | 7m | 8c |
| I-A-55 | 1n | 2c | 3a | 4f | 5c | 6d | 7k | 8b |
| I-A-56 | 1n | 2g | 3a | 4f | 5b | 6f | 7l | 8h |
| I-A-57 | 1n | 2s | 3i | — | — | — | 7m | 8c |
| I-A-58 | 1q | 2c | 3a | 4f | 5c | 6d | 7k | 8b |
| I-A-59 | 1q | 2g | 3a | 4f | 5b | 6f | 7l | 8h |
| I-A-60 | 1q | 2s | 3i | — | — | — | 7m | 8c |

In another embodiment of the first aspect, the compound of Formula (I) is according to Formula (II)

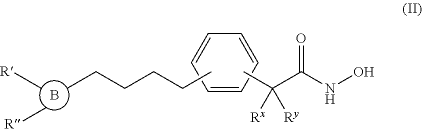

or an N-oxide, hydrate, solvate, pharmaceutically acceptable salt, agricultural formulation, prodrug or complex thereof, wherein

B is aryl, heteroaryl, heterocyclic or cycloalkyl;

R′ and R″ are each independently hydrogen, alkoxy, hydroxyl, alkyl, amino, halogen, polyether, —C(O)NR¹R², —O-alkyl-NR¹R², or CH₂C(O)NHOH where

R¹ and R² combine with the nitrogen to which they are attached to form an optionally substituted heterocyclic ring; or

R′ and R″ occur on adjacent carbon atoms and combine to form a fused 1-methyl-2,3-dihydro-1H-pyrrole;

the butyl group is optionally and independently substituted at one or more positions with one or more alkyl, halo or hydroxyl groups, or one oxo, amino or imino group; and

R^{x} and R^{y} are each independently hydrogen or alkyl;

provided that when R^{x} and R^{Y} are hydrogen and the butyl group is unsubstituted, B is not 1-H-indole; and

when B is phenyl and the butyl group is unsubstituted, R^{x}, R^{Y}, R′ and R″ are not all hydrogen.

The invention further comprises subgenera of Formula (II) in which the substituents are selected as any and all combinations of one or more of structural Formula (II), B, R′, R″, R′, R², R^{x}, and R^{y} as defined herein, including without limitation, the following:

B in Formula (II) is Selected from One of the Following Groups (9a)-(9h):

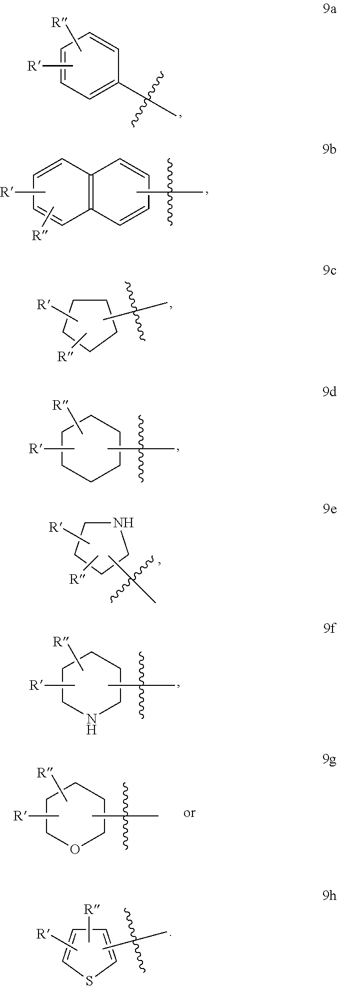

R′ and R″ in Formula (II) are Selected from One of the Following Groups (10a)-(10k):
(10a) R′ and R″ are each independently hydrogen, alkoxy, hydroxyl, alkyl, amino, halogen, polyether, —O-alkyl-NR¹R², or —CH₂C(O)NHOH where R¹ and R² are as described below, or R′ and R″ occur on adjacent carbon atoms and combine to form a fused 1-methyl-2,3-dihydro-1H-pyrrole.
(10b) R′ and R″ are each independently hydrogen, alkoxy, hydroxyl, alkyl, amino, halogen, or

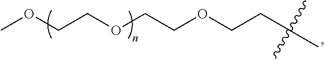

where n is 1-20.
(10c) R′ and R″ are each independently hydrogen, methoxy, ethoxy, hydroxyl, methyl, ethyl, fluoro, chloro, or bromo.
(10d) R′ and R″ occur on adjacent carbon atoms and combine to form a fused 1-methyl-2,3-dihydro-1H-pyrrole.
(10e) R′ is hydrogen and R″ is alkoxy, hydroxyl, alkyl, amino, halogen, carboxylic, N-hydroxyacetamide, phenyl, or polyether.
(10f) R′ is hydrogen and R″ is alkoxy, hydroxyl, alkyl, amino, halogen, carboxylic, N-hydroxyacetamide, or

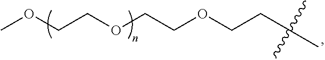

where n is 2-5.
(10g) R′ is hydrogen and R″ is methoxy, ethoxy, hydroxyl, methyl, ethyl, fluoro, chloro, or bromo.
(10h) R′ is hydrogen and R″ is

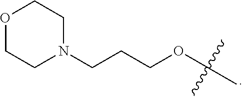 [SMILES: *OCCCN1CCOCC1]

(10i) R′ and R″ are both fluoro, hydroxyl, or methoxy.
(10j) R′ and R″ are both hydrogen.
(10k) R′ and R″ combine with group 9a, where B is substituted phenyl, to form:

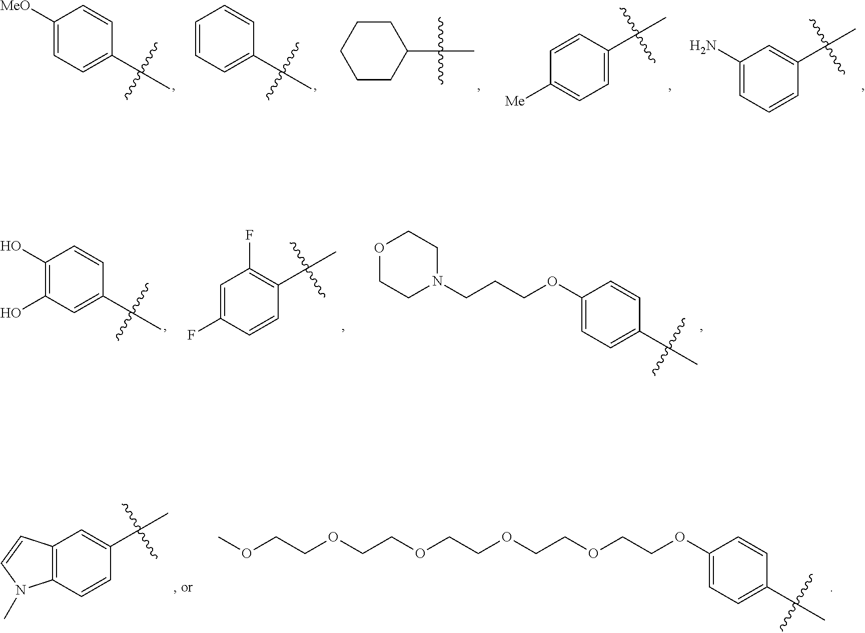

R¹ and R² in Formula (II) are Selected from One of the Following Groups (11a)-(11d):
(11a) R¹ and R² combine with the nitrogen to which they are attached to form an optionally substituted heterocyclic ring.
(11b) R¹ and R² combine with the nitrogen to which they are attached to form morpholine, pyrrolidine, piperazine, piperidine,

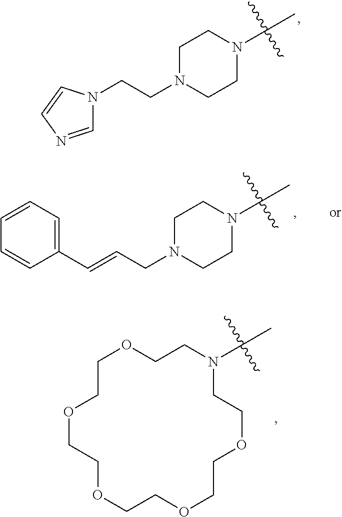 [SMILES: *N1CCN(CCn2ccnc2)CC1 ; *N1CCN(C/C=C/c2ccccc2)CC1 ; *N1CCOCCOCCOCCOCCOCC1]

each of which are optionally substituted.
(11c) R¹ and R² combine with the nitrogen to which they are attached to form

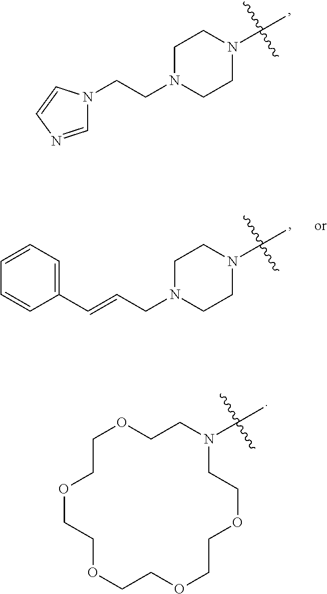

(11d) R¹ and R² combine with the nitrogen to which they are attached to form

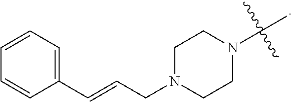

The butyl group in Formula (II) is selected from one of the following groups (12a)-(12h):
(12a) The butyl group is optionally and independently substituted at one or more positions with one or more alkyl, halo or hydroxyl groups, or one oxo, amino or imino group
(12b) The butyl group is unsubstituted.
(12c) The butyl group is substituted at one or more positions with an oxo or imino group.
(12d) The butyl group is substituted at one or more positions with one alkyl, halo or hydroxyl group.
(12e) The butyl group is substituted at one position with one alkyl, halo or hydroxyl group.
(12f) The butyl group is substituted at one position with one methyl, ethyl, fluoro, chloro, or bromo groups.
(12g) The butyl group is substituted at one position with two methyl, ethyl, fluoro, chloro, or bromo groups.
(12h) The butyl group is of the structure:

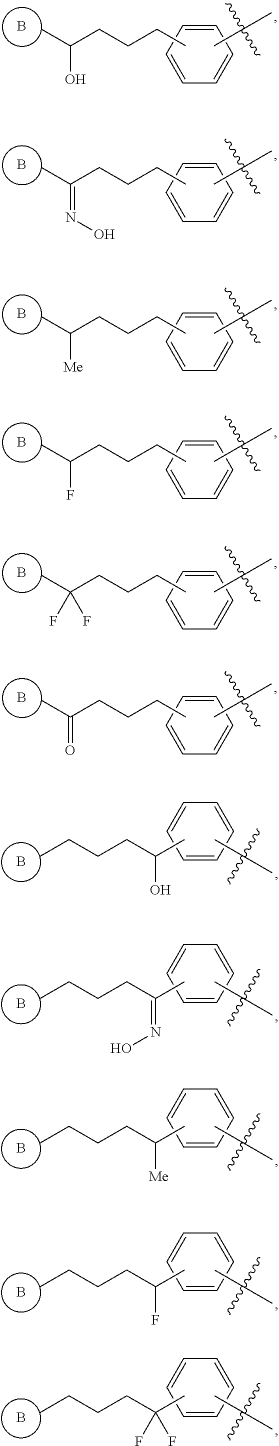

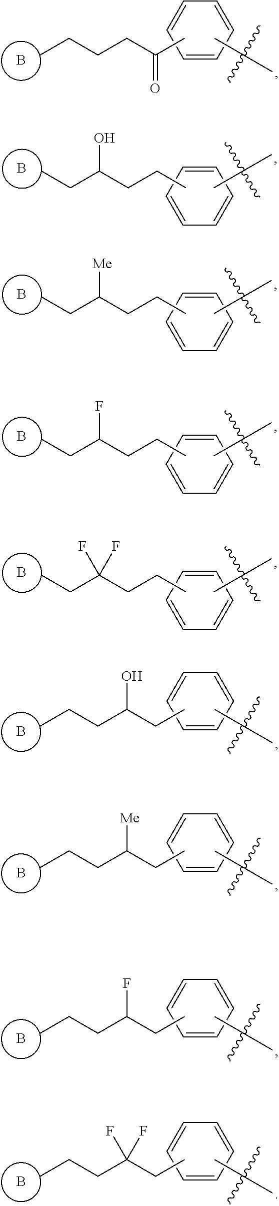

R^{x} and R^{y} in Formula (II) is selected from one of the following groups (13a)-(13h):
(13a) R^{x} and R^{y} are each independently hydrogen or alkyl.
(13b) R^{x} is hydrogen and R^{y} is alkyl.
(13c) R^{x} and R^{y} are both hydrogen.
(13d) R^{x} and R^{y} are both alkyl.
(13e) R^{x} is hydrogen and R^{y} is methyl, ethyl, propyl, isopropyl, butyl, or tert-butyl.
(13f) R^{x} is hydrogen and R^{y} is methyl.
(13g) R^{x} and R^{y} are both methyl, ethyl, propyl, or butyl.
(13h) R^{x} and R^{y} are both methyl.

Particular embodiments of the first aspect of the invention include compounds of Formula (II) composed of any combination of chemical groups as defined. These compounds are represented by Formulae II-A-1-36 in Table 2, wherein each entry is a group number as defined above (e.g., (13c) refers to R^{x} and R^{y} both being hydrogen). A dash “-” indicates that the variable is as defined in Formula (I) or defined according to any one of the applicable variable definitions (9a)-(13h) [e.g., when B is a dash, it can be either as defined for Formula (II) or any one of definitions (9a)-(9g)].

TABLE 2
Embodiments of Formula (II).

| Formula | B | butyl | R′/R″ | R¹/R² | R^{x}/R^{y} |
|---|---|---|---|---|---|
| II-A-1 | 9a | 12a | 10a | 11b | 13c |
| II-A-2 | 9a | 12b | 10a | 11d | 13f |
| II-A-3 | 9a | 12h | 10e | — | 13h |
| II-A-4 | 9c | 12a | 10a | 11b | 13c |
| II-A-5 | 9c | 12b | 10a | 11d | 13f |
| II-A-6 | 9c | 12h | 10e | — | 13h |
| II-A-7 | 9d | 12a | 10a | 11b | 13c |
| II-A-8 | 9d | 12b | 10a | 11d | 13f |
| II-A-9 | 9d | 12h | 10e | — | 13h |
| II-A-10 | 9a | 12a | 10a | 11d | 13c |
| II-A-11 | 9a | 12b | 10a | 11b | 13f |
| II-A-12 | 9a | 12h | 10e | — | 13h |
| II-A-13 | 9c | 12a | 10a | 11d | 13c |
| II-A-14 | 9c | 12b | 10a | 11b | 13f |
| II-A-15 | 9c | 12h | 10e | — | 13h |
| II-A-16 | 9d | 12a | 10a | 11d | 13c |
| II-A-17 | 9d | 12b | 10a | 11b | 13f |
| II-A-18 | 9d | 12h | 10e | — | 13h |
| II-A-19 | 9a | 12a | 10a | 11d | 13f |
| II-A-20 | 9a | 12b | 10a | 11b | 13c |
| II-A-21 | 9a | 12h | 10e | — | 13h |
| II-A-22 | 9c | 12a | 10a | 11d | 13f |
| II-A-23 | 9c | 12b | 10a | 11b | 13c |
| II-A-24 | 9c | 12h | 10e | — | 13h |
| II-A-25 | 9d | 12a | 10a | 11d | 13f |
| II-A-26 | 9d | 12b | 10a | 11b | 13c |
| II-A-27 | 9d | 12h | 10e | — | 13h |
| II-A-28 | 9a | 12a | 10a | 11d | 13h |
| II-A-29 | 9a | 12b | 10a | 11b | 13c |
| II-A-30 | 9a | 12h | 10e | — | 13f |
| II-A-31 | 9c | 12a | 10a | 11d | 13h |
| II-A-32 | 9c | 12b | 10a | 11b | 13c |
| II-A-33 | 9c | 12h | 10e | — | 13f |
| II-A-34 | 9d | 12a | 10a | 11d | 13h |
| II-A-35 | 9d | 12b | 10a | 11b | 13c |
| II-A-36 | 9d | 12h | 10e | — | 13f |
| II-A-37 | — | 12a | 10k | — | 13c |
| II-A-38 | — | 12b | 10k | — | 13f |
| II-A-39 | — | 12h | 10k | — | 13h |
| II-A-40 | — | 12a | 10k | — | 13c |
| II-A-41 | — | 12b | 10k | — | 13f |
| II-A-42 | — | 12h | 10k | — | 13h |
| II-A-43 | — | 12a | 10k | — | 13c |
| II-A-44 | — | 12b | 10k | — | 13f |
| II-A-45 | — | 12h | 10k | — | 13h |

In another embodiment of the first aspect, Formula (I) represents a prodrug of Formula (A-I), and Formula (II) represents a prodrug of Formula (A-II):

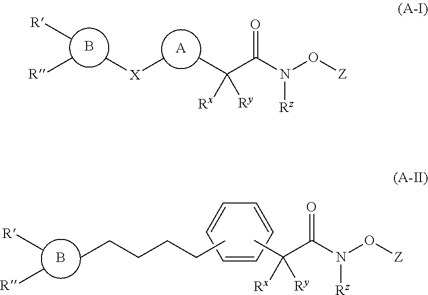

wherein

A, B, X, R′, R″, R^{x}, and R^{y} for Formula (A-I) are as defined for Formula (I);

B, R′, R″, R^{x}, R^{y} and the butyl group for Formula (A-II) are as defined for Formula (II);

R^{z} for Formulae (A-I) and (A-II) is H or —OH;

Z for Formulae (A-I) and (A-II) is R²⁰, —OR²⁰, R²¹, or

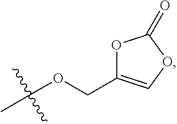

wherein R²⁰ is selected from the group consisting of —C(O)R¹⁰, —C(O)OR¹⁰, R¹¹, —CH(R¹²)OC(O)R¹⁰, —C(O)[C(R¹⁰)(R^{10′})]₁₋₄NH(R¹³), —S(O₂)R¹⁰, —P(O)(OR¹⁰)(OR¹⁰), —C(O)(CH₂)ₙCH(OH)CH₂OR¹⁰, —C(O)O(CH₂)ₙCH(OH)CH₂OR¹⁰ and —C(O)(CH₂)ₙC(O)OR¹⁰, provided that the N to which Z is bound is not directly bound to two oxygen atoms; or

R^{z} is absent and R²⁰ forms an optionally substituted heterocyclic ring with the N to which it is attached;

n is 1-4;

R¹⁰ is selected from the group consisting of hydrogen, optionally substituted C₁-C₂₀ alkyl, optionally substituted C₂-C₂₀ alkenyl, optionally substituted C₂-C₂₀ alkynyl, optionally substituted C₁-C₂₀ alkoxycarbonyl, optionally substituted cycloalkyl, optionally substituted heterocycloalkyl, optionally substituted aryl, optionally substituted heteroaryl, optionally substituted cycloalkylalkyl, optionally substituted heterocycloalkylalkyl, optionally substituted arylalkyl, optionally substituted heteroarylalkyl, optionally substituted cycloalkylalkenyl, optionally substituted heterocycloalkylalkenyl, optionally substituted arylalkenyl, optionally substituted heteroarylalkenyl, optionally substituted cycloalkylalkynyl, optionally substituted heterocycloalkylalkynyl, optionally substituted arylalkynl, optionally substituted heteroarylalkynyl, a sugar residue and an amino acid residue (preferably bonded through the carboxy terminus of the amino acid); or

R^{10′} is hydrogen; or

R¹⁰ and R^{10′} together with the carbon atom to which they are attached form an optionally substituted spirocycloalkyl;

R²¹ is -(amino acid)-R¹³, wherein R¹³ is covalently bound to the N-terminus;

R¹¹ is selected from the group consisting of hydrogen, optionally substituted alkyl, optionally substituted cycloalkyl, optionally substituted heterocycloalkyl, optionally substituted aryl, and optionally substituted heteroaryl;

R¹² is selected from hydrogen or alkyl; and

R¹³ is selected from the group consisting of hydrogen, an amino protecting group and R¹⁰;

with the provisos that when x is 4, n is not 2, and when x is 3, n is not 3.

In certain embodiments, Z is —O—C(O)—R¹⁰, —O—C(O)-[C(R¹⁰)(R^{10′})]₁₋₄—NH(R¹³) or —OR¹¹.

In other embodiments, the amino acid is an L-amino acid.

In another embodiment, the sugar residue is a saccharide selected from the group consisting of glucose, galactose, mannose, gulose, idose, talose, allose, altrose, fructose, rhamnose, ribose and xylose.

In one embodiment of the compounds according to the present invention, the prodrug is selected from the group where R^{z} is hydrogen and Z is of the structure:

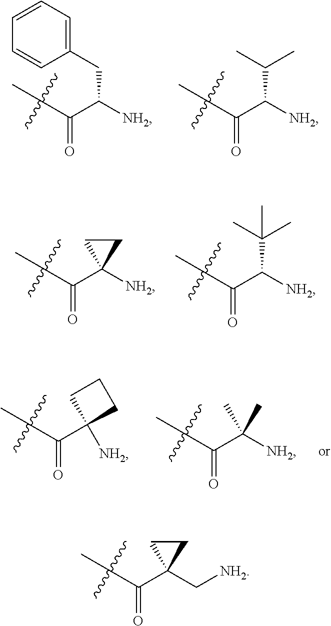

In other embodiments of the first aspect, naturally-occurring or non-naturally occurring amino acids are used to prepare the prodrugs of the invention. In particular, standard amino acids suitable as a prodrug moiety include valine, leucine, isoleucine, methionine, phenylalanine, asparagine, glutamic acid, glutamine, histidine, lysine, arginine, aspartic acid, glycine, alanine, serine, threonine, tyrosine, tryptophan, cysteine and proline, particularly the L isomers. Optionally an included amino acid is an α-, β-, or γ-amino acid. Also, naturally-occurring, non-standard amino acids can be utilized in the compositions and methods of the invention. For example, in addition to the standard naturally occurring amino acids commonly found in proteins, naturally occurring amino acids also illustratively include 4-hydroxyproline, δ-carboxyglutamic acid, selenocysteine, desmosine, 6-N-methyllysine, ε-N,N,N-trimethyllysine, 3-methylhistidine, O-phosphoserine, 5-hydroxylysine, δ-N-acetyllysine, θ-N-methylarginine, N-acetylserine, δ-aminobutyric acid, citrulline, ornithine, azaserine, homocysteine, β-cyanoalanine and S-adenosylmethionine. Non-naturally occurring amino acids include phenyl glycine, meta-tyrosine, para-amino phenylalanine, 3-(3-pyridyl)-L-alanine-, 4-(trifluoromethyl)-D-phenylalanine, and the like.

In other embodiments, the compounds of invention comprise those of Formulae (A-I) and (A-II) as defined above, except that R²⁰ of Z is described in U.S. Pat. No. 4,443,435 (incorporated by reference in its entirety) as comprising —CH(R¹³⁰)—X¹⁵—C(O)—R¹³¹ wherein

X¹⁵ is O, S, or NR¹³²;

R¹³¹ is

(a) straight or branched chain alkyl having from 1 to 20 carbon atoms especially methyl, ethyl, isopropyl, t-butyl, pentyl or hexyl;

(b) aryl having from 6 to 10 carbon atoms especially phenyl, substituted phenyl or naphthalene;

(c) cycloalkyl having from 3 to 8 carbon atoms especially cyclopentyl, or cyclohexyl;

(d) alkenyl having from 2-20 carbon atoms especially C₂-6 alkenyl such as vinyl, allyl, or butenyl;

(e) cycloalkenyl having from 5 to 8 carbon atoms especially cyclopentenyl or cyclohexenyl;

(f) alkynyl having from 2 to 20 carbon atoms especially C₂-6 alkynyl for example, ethynyl, propynyl or hexynyl;

(g) aralkyl, alkaryl, aralkenyl, aralkynyl, alkenylaryl or alkynylaryl wherein alkyl, aryl, alkenyl and alkynyl are as previously defined;

(h) loweralkoxycarbonyl especially C₁₋₆ alkoxycarbonyl such as methoxycarbonyl, ethoxycarbonyl, t-butoxycarbonyl and cyclopentoxycarbonyl;

(i) carboxyalkyl or alkanoyloxyalkyl especially carboxy-C₁₋₆ alkyl such as formyloxymethyl and formyloxypropyl; or C₁₋₆ (alkylcarboxyalkyl) such as acetoxymethyl, n-propanoyloxyethyl and pentanoyloxybutyl;

(j) saturated or unsaturated monoheterocyclic or polyheterocyclic, or fused heterocyclic, either directly bonded to the carbonyl function or linked thereto via an alkylene bridge, containing from 1 to 3 of any one or more of the heteroatoms N, S or O in each heterocyclic ring thereof and each such ring being from 3- to 8-membered; and

(k) mono- or polysubstituted derivatives of the above, each of said substituents being selected from the group consisting of lower alkyl; lower alkoxy; lower alkanoyl; lower alkanoyloxy; halo especially bromo, chloro, or fluoro; haloloweralkyl especially fluoro, chloro or bromoloweralkyl such as trifluoromethyl and 1-chloropropyl; cyano; carbethoxy; loweralkylthio, especially C₁₋₆ loweralkylthio such as methylthio, ethylthio and n-propylthio; nitro; carboxyl; amino; loweralkylamino especially C₁₋₆ alkylamino, for example, methylamino, ethylamino and n-butylamino; diloweralkylamino especially di(C₁₋₆ loweralkyl)amino such as N,N-dimethylamino, N,N-diethylamino and N,N-dihexylamino; carbamyl; loweralkylcarbamyl especially C₁₋₆ alkylcarbamyl such as methylcarbamyl and ethyl carbamoyl; and R¹³³—X—C(O)-phenyl-, wherein R¹³³ is hydrogen or alkyl having from 1 to 10 carbons;

R¹³⁰ is hydrogen, (b) R¹³¹, lower alkanoyl, cyano, haloloweralkyl, carbamyl, loweralkylcarbamyl, or diloweralkylcarbamyl, —CH₂ONO₂, or —CH₂OCOR¹³¹;

R¹³² is hydrogen or lower alkyl; or

wherein R¹³¹ and R¹³⁰ may be taken together to form a ring cyclizing moiety selected from the group consisting of:

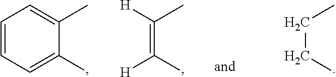

In other embodiments, the compounds of invention comprise those of Formulae (A-I) and (A-II) as defined above, except that R²⁰ of Z is described in U.S. Pat. No. 6,407,235 (incorporated by reference in its entirety) as comprising:

a) —C(O)(CH₂)ₘC(O)OR⁴⁰, wherein m is 1, 2, 3 or 4,

b)

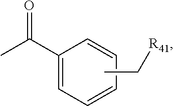

wherein R⁴¹ is —N(R⁴²)(R⁴³) and R⁴² and R⁴³ are hydrogen or lower alkyl, or is a five or six member heterocyclyl or heteroaryl optionally substituted by lower alkyl, or

c) —C(O)(CH₂)NHC(O)(CH₂)N(R⁴²)(R⁴³).

In other embodiments, the compounds of invention comprise those of Formulae (A) and (B) as defined above, except that R²⁰ of Z (of Formula A) and Z¹ (of Formula B) is described in U.S. Pat. No. 6,545,131 (incorporated by reference in its entirety) as comprising:

CO—(CH═CH)ₙ₁—(CH₂)ₙ₂—Ar—NH₂, —CO—(CH₂)ₙ₂—(CH═CH)ₙ₁—Ar—NH₂, CO—(CH₂)ₙ₂—(CH═CH)ₙ₁—CO—NH—Ar—NH₂ and CO—(CH═CH)ₙ₁—(CH₂)ₙ₂—CO—NH—Ar—NH₂ and substituted variations thereof, where n1 and n2 are from 0 to 5, Ar is a substituted or unsubstituted aryl group. In some embodiments, Z is CO—(CH₂)ₙ₃—NH₂, where n3 is from 0 to 15, preferably 3-15, and also preferably 6-12. Particularly, substituent groups within this class are 6-aminohexanoyl, 7-aminoheptanoyl, 8-aminooctanoyl, 9-aminononanoyl, 10-aminodecanoyl, 11-aminoundecanoyl, and 12-aminododecanoyl. These substituents are generally synthesized from the corresponding amino acids, for example, 6-aminohexanoic acid. The amino acids are N-terminal protected by standard methods, for example Boc protection. Dicyclohexylcarbodiimide (DCC)-promoted coupling of the N-terminal protected substituent to thapsigargin, followed by standard deprotection reactions produces primary amine-containing thapsigargin analogs.

In other embodiments, the compounds of invention comprise those of Formulae (A-I) and (A-II) as defined above, except that R²⁰ of Z is described in U.S. Pat. No. 7,115,573 (incorporated by reference in its entirety) as comprising:

(a) an oligopeptide of the formula (AA)ₙ-AA³-AA²-AA¹, wherein: each AA independently represents an amino acid, n is 0 or 1, and when n is 1, then (AA)ₙ is AA⁴ which represents any amino acid, AA³ represents isoleucine, AA² represents any amino acid, and AA′ represents any amino acid;

(b) a stabilizing group, and

(c) optionally, a linker group not cleavable by a trouase, such as TOP (described in greater detail below);

wherein

the oligopeptide is directly linked to the stabilizing group at a first attachment site of the oligopeptide and the oligopeptide is directly linked to the therapeutic agent or indirectly linked through the linker group to the therapeutic agent at a second attachment site of the oligopeptide;

the stabilizing group hinders cleavage of the compound by enzymes present in whole blood; and

the compound is cleavable by an enzyme associated with the target cell, the enzyme associated with the target cell being other than TOP (Thimet oligopeptidase). The compound preferably includes an oligopeptide that is resistant to cleavage by a trouase, particularly TOP, i.e., resistant to cleavage under physiological conditions. The optionally present linker group that is not cleavable by a trouase is not cleavable under physiological conditions.

The typical orientation of these portions of the prodrug is as follows: (stabilizing group)-(oligopeptide)-(optional linker group)-(therapeutic agent).

Direct linkage of two portions of the prodrug means a covalent bond exists between the two portions. The stabilizing group and the oligopeptide are therefore directly linked via a covalent chemical bond at the first attachment site of the oligopeptide, typically the N-terminus of the oligopeptide. When the oligopeptide and the therapeutic agent are directly linked then they are covalently bound to one another at the second attachment site of the oligopeptide. The second attachment site of the oligopeptide is typically the C-terminus of the oligopeptide, but may be elsewhere on the oligopeptide.

Indirect linkage of two portions of the prodrug means each of the two portions is covalently bound to a linker group. In an alternative embodiment, the prodrug has indirect linkage of the oligopeptide to the therapeutic agent. Thus, typically, the oligopeptide is covalently bound to the linker group which, in turn, is covalently bound to the therapeutic agent.

In an alternative embodiment, the orientation of the prodrug may be reversed so that a stabilizing group is attached to the oligopeptide at the C-terminus and the therapeutic agent is directly or indirectly linked to the N-terminus of the oligopeptide. Thus, in an alternative embodiment, the first attachment site of the oligopeptide may be the C-terminus of the oligopeptide and the second attachment site by the oligopeptide may be the N-terminus of the oligopeptide. The linker group may optimally be present between the therapeutic agent and the oligopeptide. The alternative embodiment of the prodrug of the invention functions in the same manner as does the primary embodiment.

The stabilizing group typically protects the prodrug from cleavage by proteinases and peptidases present in blood, blood serum, and normal tissue. Particularly, since the stabilizing group caps the N-terminus of the oligopeptide, and is therefore sometimes referred to as an N-cap or N-block, it serves to ward against peptidases to which the prodrug may otherwise be susceptible. A stabilizing group that hinders cleavage of the oligopeptide by enzymes present in whole blood is chosen from the following:

(a) other than an amino acid, and

(b) an amino acid that is either
(i) a non-genetically-encoded amino acid or
(ii) aspartic acid or glutamic acid attached to the N-terminus of the oligopeptide at the β-carboxyl group of aspartic acid or the γ-carboxyl group of glutamic acid.

For example, dicarboxylic (or a higher order carboxylic) acid or a pharmaceutically acceptable salt thereof may be used as a stabilizing group. Since chemical radicals having more than two carboxylic acids are also acceptable as part of the prodrug, the end group having dicarboxylic (or higher order carboxylic) acids is an exemplary N-cap. The N-cap may thus be a monoamide derivative of a chemical radical containing two or more carboxylic acids where the amide is attached onto the amino terminus of the peptide and the remaining carboxylic acids are free and uncoupled. For this purpose, the N-cap is preferably succinic acid, adipic acid, glutaric acid, or phthalic acid, with succinic acid and adipic acid being most preferred. Other examples of useful N-caps in the prodrug compound of the invention include diglycolic acid, fumaric acid, naphthalene dicarboxylic acid, pyroglutamic acid, acetic acid, 1- or 2-, naphthylcarboxylic acid, 1,8-naphthyl dicarboxylic acid, aconitic acid, carboxycinnamic acid, triazole dicarboxylic acid, gluconic acid, 4-carboxyphenyl boronic acid, a (PEG).sub.n-analog such as polyethylene glycolic acid, butane disulfonic acid, maleic acid, nipecotic acid, and isonipecotic acid.

Further, a non-genetically encoded amino acid such as one of the following may also be used as the stabilizing group: β-alanine, thiazolidine-4-carboxylic acid, 2-thienylalanine, 2-naphthylalanine, D-alanine, D-leucine, D-methionine, D-phenylalanine, 3-amino-3-phenylpropionic acid, γ-aminobutyric acid, 3-amino-4,4-diphenylbutyric acid, tetrahydroisoquinoline-3-carboxylic acid, 4-aminomethylbenzoic acid, and aminoisobutyric acid.

A linker group between the oligopeptide and the therapeutic agent may be advantageous for reasons such as the following:

(a) As a spacer for steric considerations in order to facilitate enzymatic release of the AA′ amino acid or other enzymatic activation steps;

(b) To provide an appropriate attachment chemistry between the therapeutic agent and the oligopeptide;

(c) To improve the synthetic process of making the prodrug conjugate (e.g., by pre-derivitizing the therapeutic agent or oligopeptide with the linker group before conjugation to enhance yield or specificity);

(d) To improve physical properties of the prodrug;

(e) To provide an additional mechanism for intracellular release of the drug.

Linker structures are dictated by the required functionality. Examples of potential linker chemistries are hydrazide, ester, ether, and sulfhydryl. Aminocaproic acid is an example of a bifunctional linker group. When aminocaproic acid is used as part of the linker group, it is not counted as an amino acid in the numbering scheme of the oligopeptide.

The oligopeptide moiety is linked at a first attachment site of the oligopeptide to a stabilizing group that hinders cleavage of the oligopeptide by enzymes present in whole blood, and directly or indirectly linked to a therapeutic agent at a second attachment site of the oligopeptide. The linkage of the oligopeptide to the therapeutic agent and the stabilizing group may be performed in any order or concurrently. The resulting conjugate is tested for cleavability by TOP. Test compounds resistant to cleavage by TOP are selected. The resulting conjugate may also be tested for stability in whole blood. Test compounds stable in whole blood are selected.

The combination of oligopeptide, stabilizing group, and optional linker of U.S. Pat. No. 7,115,573 is further described in US 2002-0142955, also incorporated herein by reference.

In other embodiments, the compounds of invention comprise those of Formulae (A-I) and (A-II) as defined above, except that R²⁰ of Z is described in US 2004-0019017 A1 (incorporated by reference in its entirety and which describes caspase inhibitor prodrugs), as comprising:

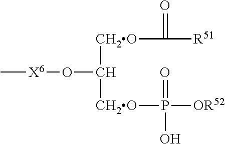

wherein

R⁵¹ is a saturated or unsaturated, straight-chain or branched, substituted or unsubstituted alkyl of 2 to 30, preferably 2 to 24, carbon atoms;

R⁵² is H or a phospholipid head group, preferably choline; and

X⁶ is a direct covalent bond or a group C(O)LR⁵³ wherein L is a saturated or unsaturated, straight-chain or branched, substituted or unsubstituted alkyl having from 2 to 15 carbon atoms, which optionally includes cyclic elements, and is optionally interrupted by one or more atoms selected from the group consisting of oxygen, sulfur and N(R⁵⁴); where
R⁵³ is selected from the group consisting of O, S and N(R⁵⁴); and
R⁵⁴ is H or a saturated or unsaturated alkyl having 1 to 6 carbon atoms.

In other embodiments, the compounds of invention comprise those of Formulae (A-I) and (A-II) as defined above, except that R²⁰ of Z is the Y moiety described in U.S. Pat. No. 7,115,573 (incorporated by reference in its entirety).

In other embodiments, the compounds of invention comprise those of Formulae (A-I) and (A-II) as defined above, except that R²⁰ of Z is described in US 2006-0166903 A1 (incorporated by reference in its entirety, as comprising-X-L-O—P(O)(O⁻)—O—CH₂—CH₂—N(CH₃)₃⁺, wherein X and L are as described in US 2006-0166903A1.

In other embodiments, the compounds of the invention comprise those of Formulae (A-I) and (A-II) as defined above, except Z is one of the cleavable prodrug moieties described in U.S. Pat. No. 6,855,702, US 2005-0137141, and US 2006-0135594, all hereby incorporated by reference in their entirety.

In certain embodiments of the first aspect, the compound of the invention is one of the compounds of Table 3, and certain embodiments of the invention are compositions comprising a compound of Table 3:

| | Structure | Name |
|---|---|---|
| 1 | 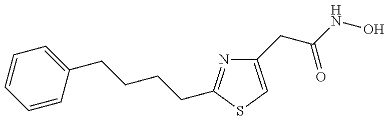 [SMILES: O=C(Cc1csc(CCCCc2ccccc2)n1)NO] | N-hydroxy-2-(2-(4-phenylbutyl)thiazol-4-yl)acetamide |
| 2 | 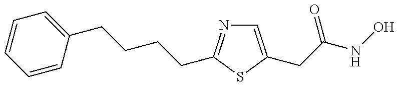 [SMILES: O=C(Cc1cnc(CCCCc2ccccc2)s1)NO] | N-hydroxy-2-(2-(4-phenylbutyl)thiazol-5-yl)acetamide |
| 3 | 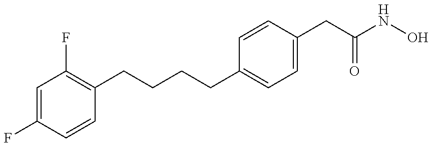 [SMILES: O=C(Cc1ccc(CCCCc2ccc(F)cc2F)cc1)NO] | 2-(4-(4-(2,4-difluorophenyl)butyl)phenyl)-N-hydroxyacetamide |
| 4 | 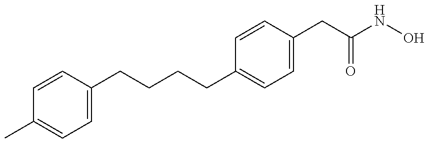 [SMILES: Cc1ccc(CCCCc2ccc(CC(=O)NO)cc2)cc1] | N-hydroxy-2-(4-(4-p-tolylbutyl)phenyl)acetamide |
| 5 | 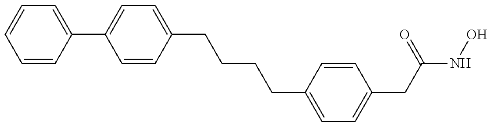 [SMILES: O=C(Cc1ccc(CCCCc2ccc(-c3ccccc3)cc2)cc1)NO] | 2-(4-(4-(biphenyl-4-yl)butyl)phenyl)-N-hydroxyacetamide |
| 6 | 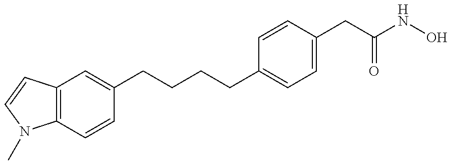 [SMILES: Cn1ccc2cc(CCCCc3ccc(CC(=O)NO)cc3)ccc21] | N-hydroxy-2-(4-(4-(1-methyl-1H-indol-5-yl)butyl)phenyl) acetamide |
| 7 | 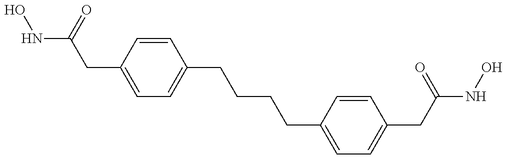 [SMILES: O=C(Cc1ccc(CCCCc2ccc(CC(=O)NO)cc2)cc1)NO] | 2,2′-(4,4′-(butane-1,4-diyl)bis(4,1-phenylene))bis(N- hydroxyacetamide) |
| 8 | 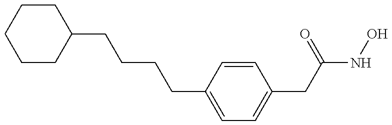 [SMILES: O=C(Cc1ccc(CCCCC2CCCCC2)cc1)NO] | 2-(4-(4-cyclohexylbutyl)phenyl)-N-hydroxyacetamide |
| 9 | 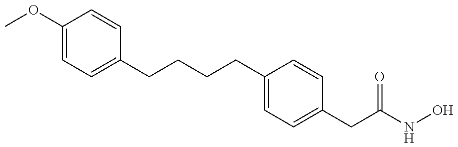 [SMILES: COc1ccc(CCCCc2ccc(CC(=O)NO)cc2)cc1] | N-hydroxy-2-(4-(4-(4-methoxyphenyl)butyl)phenyl)acetamide |
| 10 | 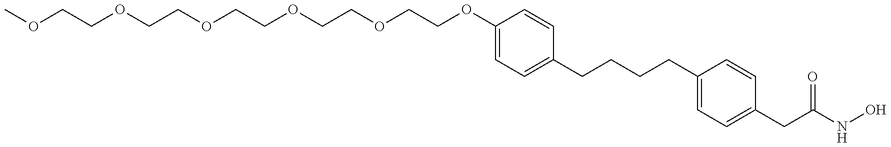 [SMILES: COCCOCCOCCOCCOCCOc1ccc(CCCCc2ccc(CC(=O)NO)cc2)cc1] | 2-(4-(4-(4-(2,5,8,11,14-pentaoxahexadecan-16-yloxy)phenyl)butyl) phenyl)-N-hydroxyacetamide |
| 11 | 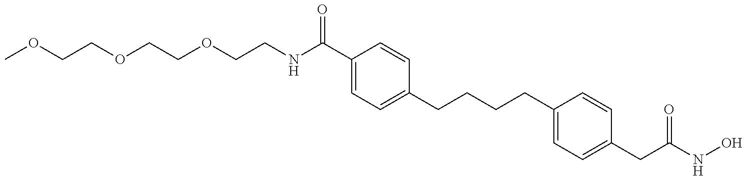 [SMILES: COCCOCCOCCNC(=O)c1ccc(CCCCc2ccc(CC(=O)NO)cc2)cc1] | 4-(4-(4-(2-(hydroxyamino)-2-oxoethyl)phenyl)butyl)-N-(2-(2-(2- methoxyethoxy)ethoxy)ethyl)benzamide |
| 12 | 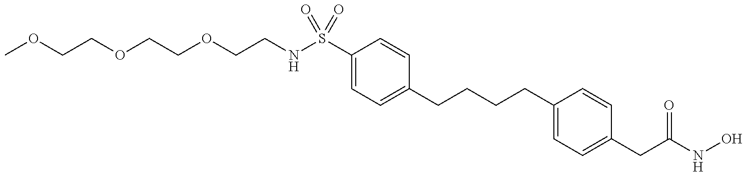 [SMILES: COCCOCCOCCNS(=O)(=O)c1ccc(CCCCc2ccc(CC(=O)NO)cc2)cc1] | N-hydroxy-2-(4-(4-(4-(N-(2-(2-(2-methoxyethoxy)ethoxy)ethyl) sulfamoyl)phenyl)butyl)phenyl)acetamide |
| 13 | 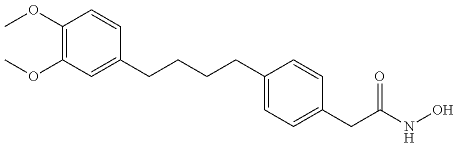 [SMILES: COc1ccc(CCCCc2ccc(CC(=O)NO)cc2)cc1OC] | 2-(4-(4-(3,4-dimethoxyphenyl)butyl)phenyl)-N-hydroxyacetamide |
| 14 | 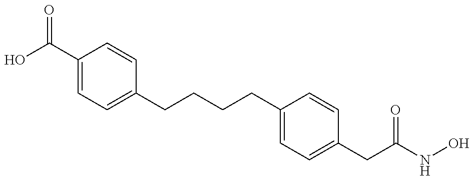 [SMILES: O=C(Cc1ccc(CCCCc2ccc(C(=O)O)cc2)cc1)NO] | 4-(4-(4-(2-(hydroxyamino)-2-oxoethyl)phenyl)butyl)benzoic acid |
| 15 | 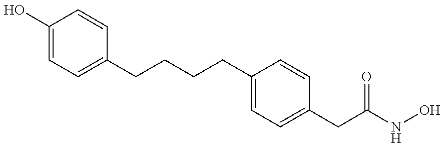 [SMILES: O=C(Cc1ccc(CCCCc2ccc(O)cc2)cc1)NO] | N-hydroxy-2-(4-(4-(4-hydroxyphenyl)butyl)phenyl)acetamide |
| 16 | 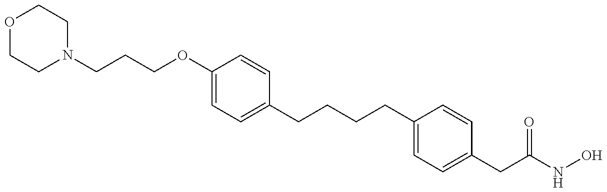 [SMILES: O=C(Cc1ccc(CCCCc2ccc(OCCCN3CCOCC3)cc2)cc1)NO] | N-hydroxy-2-(4-(4-(4-(3-morpholinopropoxy) phenyl)butyl)phenyl)acetamide |
| 17 | 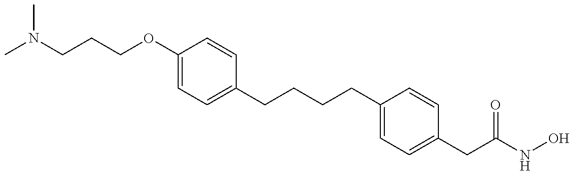 [SMILES: CN(C)CCCOc1ccc(CCCCc2ccc(CC(=O)NO)cc2)cc1] | 2-(4-(4-(4-(3-(dimethylamino)propoxy)phenyl)butyl)phe- nyl)-N-hydroxyacetamide |
| 18 | 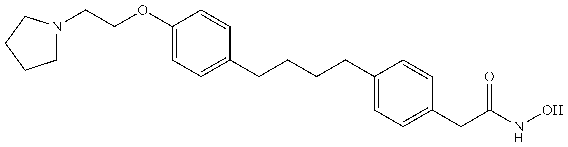 [SMILES: O=C(Cc1ccc(CCCCc2ccc(OCCN3CCCC3)cc2)cc1)NO] | N-hydroxy-2-(4-(4-(4-(2-(pyrrolidin-1-yl)ethoxy)phenyl)butyl) phenyl)acetamide |
| 19 | 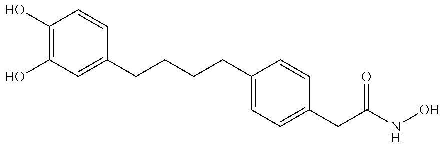 [SMILES: O=C(Cc1ccc(CCCCc2ccc(O)c(O)c2)cc1)NO] | 2-(4-(4-(3,4-dihydroxyphenyl)butyl)phenyl)-N-hydroxyacetamide |
| 20 | 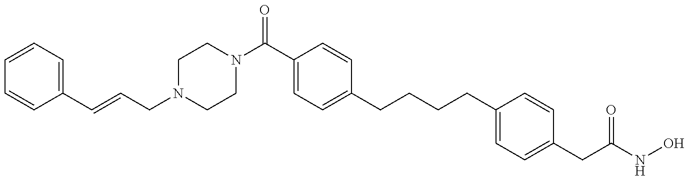 [SMILES: O=C(Cc1ccc(CCCCc2ccc(C(=O)N3CCN(C/C=C/c4ccccc4)CC3)cc2)cc1)NO] | (E)-2-(4-(4-(4-(4-cinnamylpiperazine-1-carbonyl)phenyl)butyl) phenyl)-N-hydroxyacetamide |
| 21 | 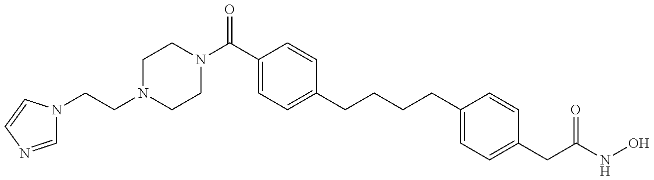 [SMILES: O=C(Cc1ccc(CCCCc2ccc(C(=O)N3CCN(CCn4ccnc4)CC3)cc2)cc1)NO] | 2-(4-(4-(4-(4-(2-(1H-imidazol-1-yl)ethyl)piperazine-1- carbonyl)phenyl)butyl)phenyl)-N-hydroxyacetamide |
| 22 | 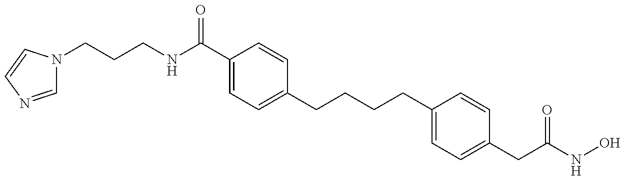 [SMILES: O=C(Cc1ccc(CCCCc2ccc(C(=O)NCCCn3ccnc3)cc2)cc1)NO] | N-(3-(1H-imidazol-1-yl)propyl)-4-(4-(4-(2-(hydroxyamino)-2- oxoethyl)phenyl)butyl)benzamide |
| 23 | 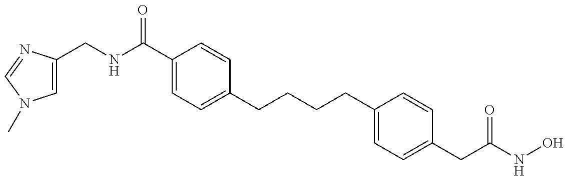 [SMILES: Cn1cnc(CNC(=O)c2ccc(CCCCc3ccc(CC(=O)NO)cc3)cc2)c1] | 4-(4-(4-(2-(hydroxyamino)-2-oxoethyl)phenyl)butyl)- N-((1-methyl-1H-imidazol-4-yl)methyl)benzamide |
| 24 | 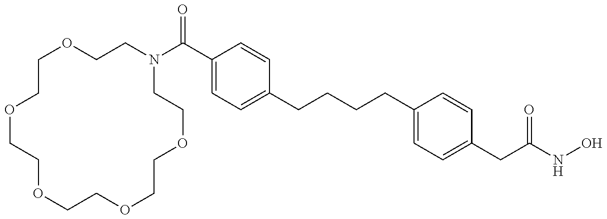 [SMILES: O=C(Cc1ccc(CCCCc2ccc(C(=O)N3CCOCCOCCOCCOCCOCC3)cc2)cc1)NO] | 2-(4-(4-(4-(1,4,7,10,13-pentaoxa-16-azacyclooctadecane- 16-carbonyl)phenyl)butyl)phenyl)-N-hydroxyacetamide |
| 25 | 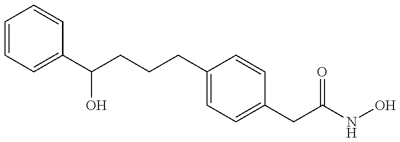 [SMILES: O=C(Cc1ccc(CCCC(O)c2ccccc2)cc1)NO] | N-hydroxy-2-(4-(4-hydroxy-4-phenylbutyl)phenyl)acetamide |
| 26 | 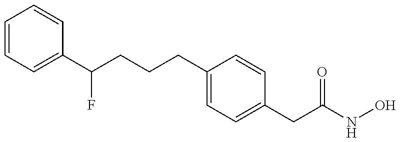 [SMILES: O=C(Cc1ccc(CCCC(F)c2ccccc2)cc1)NO] | 2-(4-(4-fluoro-4-phenylbutyl)phenyl)-N-hydroxyacetamide |
| 27 | 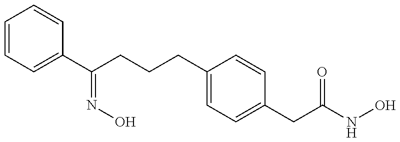 [SMILES: O=C(Cc1ccc(CCC/C(=N\O)c2ccccc2)cc1)NO] | (E)-N-hydroxy-2-(4-(4-(hydroxyimino)-4-phenylbutyl)phenyl) acetamide |
| 28 | 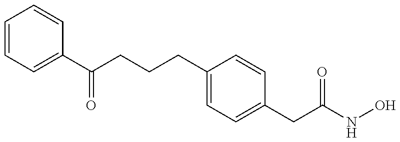 [SMILES: O=C(Cc1ccc(CCCC(=O)c2ccccc2)cc1)NO] | N-hydroxy-2-(4-(4-oxo-4-phenylbutyl)phenyl)acetamide |
| 29 | 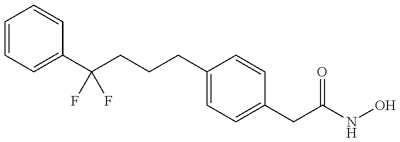 [SMILES: O=C(Cc1ccc(CCCC(F)(F)c2ccccc2)cc1)NO] | 2-(4-(4,4-difluoro-4-phenylbutyl)phenyl)-N-hydroxyacetamide |
| 30 | 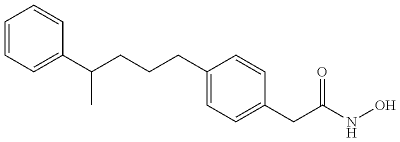 [SMILES: CC(CCCc1ccc(CC(=O)NO)cc1)c1ccccc1] | N-hydroxy-2-(4-(4-phenylpentyl)phenyl)acetamide |
| 31 | 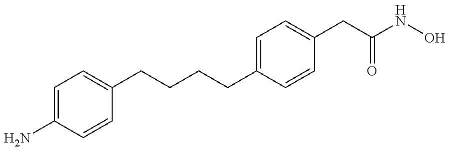 [SMILES: Nc1ccc(CCCCc2ccc(CC(=O)NO)cc2)cc1] | 2-(4-(4-(4-aminophenyl)butyl)phenyl)-N-hydroxyacetamide |
| 32 | 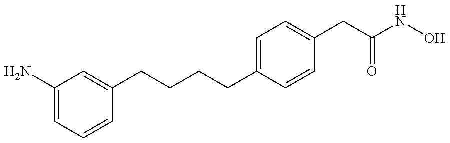 [SMILES: Nc1cccc(CCCCc2ccc(CC(=O)NO)cc2)c1] | 2-(4-(4-(3-aminophenyl)butyl)phenyl)-N-hydroxyacetamide |
| 33 | 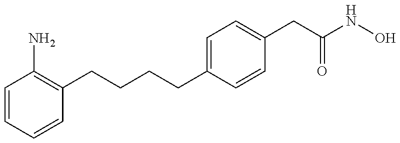 [SMILES: Nc1ccccc1CCCCc1ccc(CC(=O)NO)cc1] | 2-(4-(4-(2-aminophenyl)butyl)phenyl)-N-hydroxyacetamide |
| 34 | 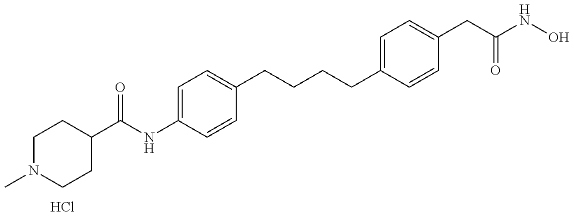 [SMILES: CN1CCC(C(=O)Nc2ccc(CCCCc3ccc(CC(=O)NO)cc3)cc2)CC1 ; Cl] | N-(4-(4-(4-(2-(hydroxyamino)-2-oxoethyl)phenyl)butyl) phenyl)-1-methylpiperidine-4-carboxamide hydrochloride |
| 35 | 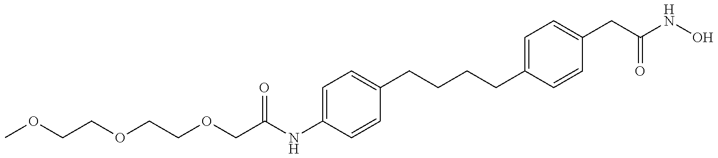 [SMILES: COCCOCCOCC(=O)Nc1ccc(CCCCc2ccc(CC(=O)NO)cc2)cc1] | N-hydroxy-2-(4-(4-(4-(2-(2-(2-methoxyethoxy)ethoxy) acetamido)phenyl)butyl)phenyl)acetamide |
| 36 | 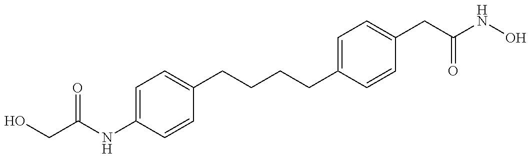 [SMILES: O=C(Cc1ccc(CCCCc2ccc(NC(=O)CO)cc2)cc1)NO] | N-hydroxy-2-(4-(4-(4-(2-hydroxyacetamido)phenyl) butyl)phenyl)acetamide |
| 37 | 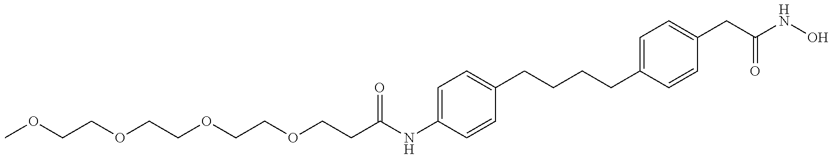 [SMILES: COCCOCCOCCOCCC(=O)Nc1ccc(CCCCc2ccc(CC(=O)NO)cc2)cc1] | N-(4-(4-(4-(2-(hydroxyamino)-2-oxoethyl)phenyl)butyl) phenyl)-2,5,8,11-tetraoxatetradecan-14-amide |
| 38 | 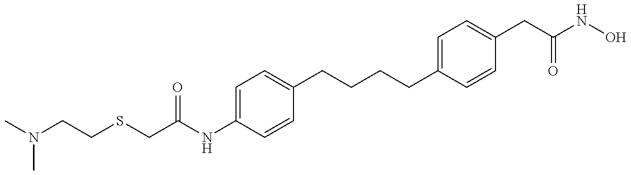 [SMILES: CN(C)CCSCC(=O)Nc1ccc(CCCCc2ccc(CC(=O)NO)cc2)cc1] | 2-(2-(dimethylamino)ethylthio)-N-(4-(4-(4-(2- (hydroxyamino)-2-oxoethyl)phenyl)butyl)phenyl)acetamide |
| 39 | 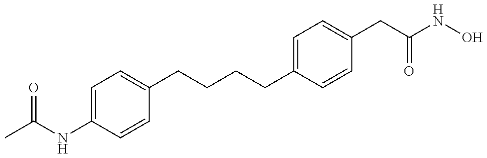 [SMILES: CC(=O)Nc1ccc(CCCCc2ccc(CC(=O)NO)cc2)cc1] | 2-(4-(4-(4-acetamidophenyl)butyl)phenyl)-N-hydroxyacetamide |
| 40 | 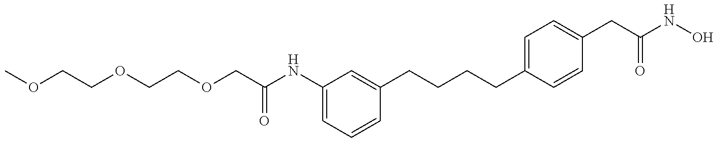 [SMILES: COCCOCCOCC(=O)Nc1cccc(CCCCc2ccc(CC(=O)NO)cc2)c1] | N-hydroxy-2-(4-(4-(3-(2-(2-(2-methoxyethoxy)ethoxy) acetamido)phenyl)butyl)phenyl)acetamide |
| 41 | 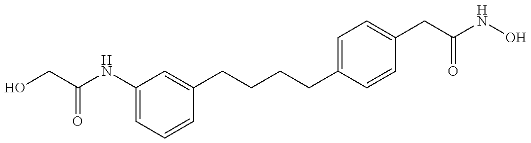 [SMILES: O=C(Cc1ccc(CCCCc2cccc(NC(=O)CO)c2)cc1)NO] | N-hydroxy-2-(4-(4-(3-(2-hydroxyacetamido)phe- nyl)butyl)phenyl)acetamide |
| 42 | 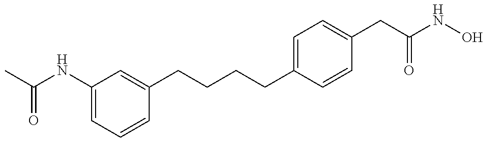 [SMILES: CC(=O)Nc1cccc(CCCCc2ccc(CC(=O)NO)cc2)c1] | 2-(4-(4-(3-acetamidophenyl)butyl)phenyl)-N-hydroxyacetamide |
| 43 | 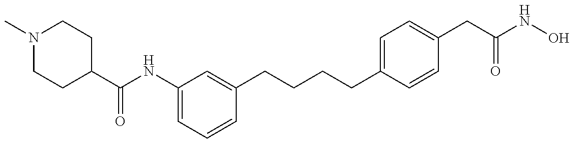 [SMILES: CN1CCC(C(=O)Nc2cccc(CCCCc3ccc(CC(=O)NO)cc3)c2)CC1] | N-(3-(4-(4-(2-(hydroxyamino)-2-oxoethyl) phenyl)butyl)phenyl)-1-methylpiperidine-4-carboxamide |
| 44 | 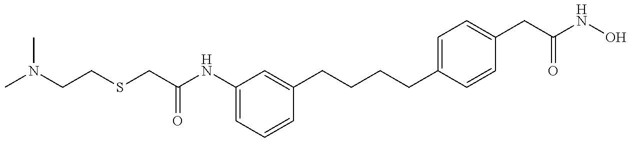 [SMILES: CN(C)CCSCC(=O)Nc1cccc(CCCCc2ccc(CC(=O)NO)cc2)c1] | 2-(2-(dimethylamino)ethylthio)-N-(3-(4-(4-(2- (hydroxyamino)-2-oxoethyl)phenyl)butyl)phenyl)acetamide |
| 45 | 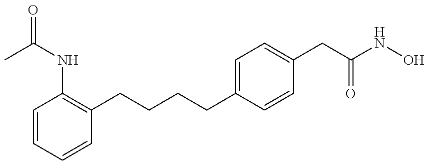 [SMILES: CC(=O)Nc1ccccc1CCCCc1ccc(CC(=O)NO)cc1] | 2-(4-(4-(2-acetamidophenyl)butyl)phenyl)-N-hydroxyacetamide |
| 46 | 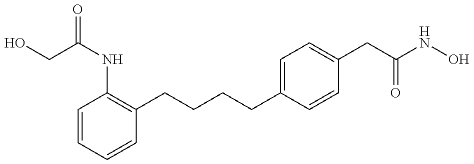 [SMILES: O=C(Cc1ccc(CCCCc2ccccc2NC(=O)CO)cc1)NO] | N-hydroxy-2-(4-(4-(2-(2-hydroxyacetamido)phe- nyl)butyl)phenyl)acetamide |
| 47 | 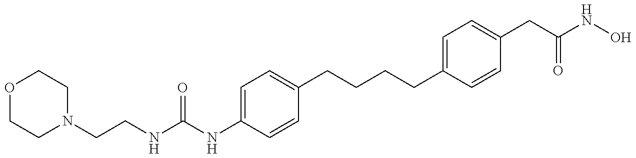 [SMILES: O=C(Cc1ccc(CCCCc2ccc(NC(=O)NCCN3CCOCC3)cc2)cc1)NO] | N-hydroxy-2-(4-(4-(4-(3-(2- morpholinoethyl)ureido)phenyl)butyl)phenyl)acetamide |
| 48 | 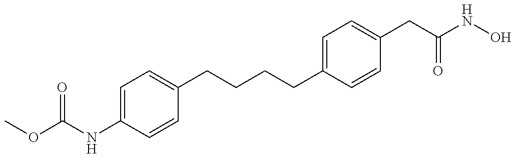 [SMILES: COC(=O)Nc1ccc(CCCCc2ccc(CC(=O)NO)cc2)cc1] | methyl 4-(4-(4-(2-(hydroxyamino)-2-oxoethyl)phenyl)butyl) phenylcarbamate |
| 49 | 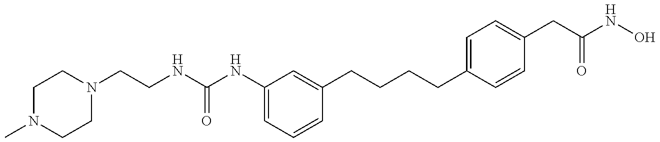 [SMILES: CN1CCN(CCNC(=O)Nc2cccc(CCCCc3ccc(CC(=O)NO)cc3)c2)CC1] | N-hydroxy-2-(4-(4-(3-(3-(2-(4-methylpiperazin-1- yl)ethyl)ureido)phenyl)butyl)phenyl)acetamide |
| 50 | 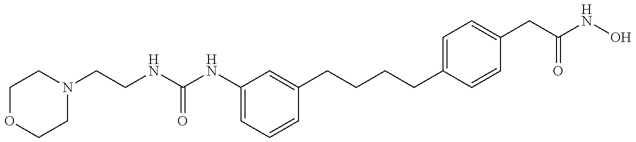 [SMILES: O=C(Cc1ccc(CCCCc2cccc(NC(=O)NCCN3CCOCC3)c2)cc1)NO] | N-hydroxy-2-(4-(4-(3-(3-(2-morpholinoethyl)urei- do)phenyl)butyl)phenyl)acetamide |
| 51 | 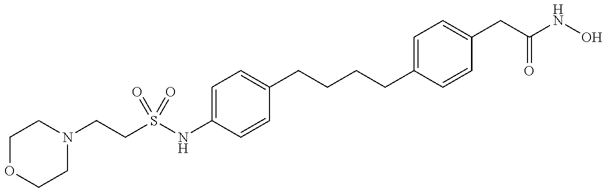 [SMILES: O=C(Cc1ccc(CCCCc2ccc(NS(=O)(=O)CCN3CCOCC3)cc2)cc1)NO] | N-hydroxy-2-(4-(4-(4-(2-morpholinoethylsulfona- mido)phenyl)butyl)phenyl)acetamide |
| 52 | 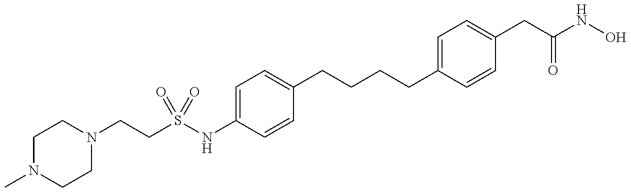 [SMILES: CN1CCN(CCS(=O)(=O)Nc2ccc(CCCCc3ccc(CC(=O)NO)cc3)cc2)CC1] | N-hydroxy-2-(4-(4-(4-(2-(4-methylpiperazin-1- yl)ethylsulfonamido)phenyl)butyl)phenyl)acetamide |
| 53 | 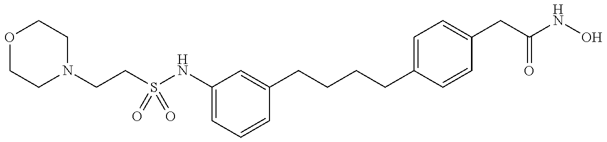 [SMILES: O=C(Cc1ccc(CCCCc2cccc(NS(=O)(=O)CCN3CCOCC3)c2)cc1)NO] | N-hydroxy-2-(4-(4-(3-(2-morpholinoethylsulfona- mido)phenyl)butyl)phenyl)acetamide |
| 54 | 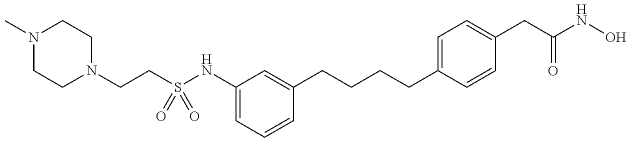 [SMILES: CN1CCN(CCS(=O)(=O)Nc2cccc(CCCCc3ccc(CC(=O)NO)cc3)c2)CC1] | N-hydroxy-2-(4-(4-(3-(2-(4-methylpiperazin-1-yl) ethylsulfonamido)phenyl)butyl)phenyl)acetamide |
| 55 | 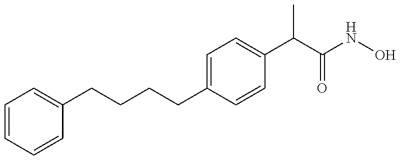 [SMILES: CC(C(=O)NO)c1ccc(CCCCc2ccccc2)cc1] | N-hydroxy-2-(4-(4-phenylbutyl)phenyl)propanamide |
| 56 | 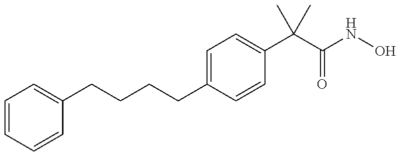 [SMILES: CC(C)(C(=O)NO)c1ccc(CCCCc2ccccc2)cc1] | N-hydroxy-2-methyl-2-(4-(4-phenylbutyl)phenyl)propanamide |
| 57 | 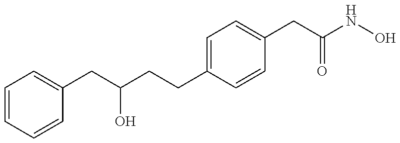 [SMILES: O=C(Cc1ccc(CCC(O)Cc2ccccc2)cc1)NO] | N-hydroxy-2-(4-(3-hydroxy-4-phenylbutyl)phenyl)acetamide |
| 58 | 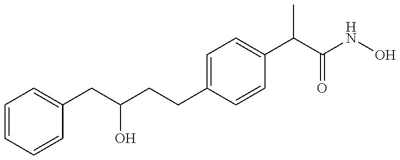 [SMILES: CC(C(=O)NO)c1ccc(CCC(O)Cc2ccccc2)cc1] | N-hydroxy-2-(4-(3-hydroxy-4-phenylbutyl)phenyl)propanamide |
| 59 | 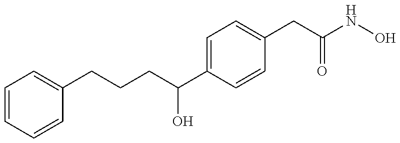 [SMILES: O=C(Cc1ccc(C(O)CCCc2ccccc2)cc1)NO] | N-hydroxy-2-(4-(1-hydroxy-4-phenylbutyl)phenyl)acetamide |
| 60 | 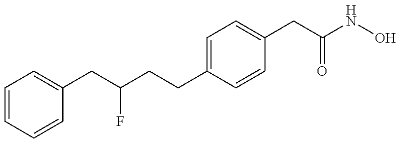 [SMILES: O=C(Cc1ccc(CCC(F)Cc2ccccc2)cc1)NO] | 2-(4-(3-fluoro-4-phenylbutyl)phenyl)-N-hydroxyacetamide |
| 61 | 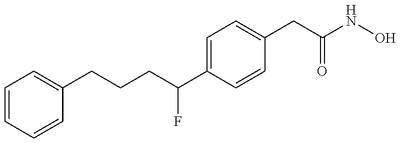 [SMILES: O=C(Cc1ccc(C(F)CCCc2ccccc2)cc1)NO] | 2-(4-(1-fluoro-4-phenylbutyl)phenyl)-N-hydroxyacetamide |
| 62 | 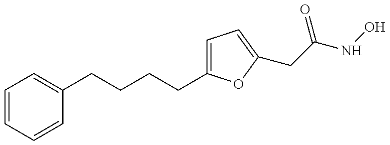 [SMILES: O=C(Cc1ccc(CCCCc2ccccc2)o1)NO] | N-hydroxy-2-(5-(4-phenylbutyl)furan-2-yl)acetamide |
| 63 | 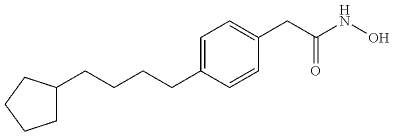 [SMILES: O=C(Cc1ccc(CCCCC2CCCC2)cc1)NO] | 2-(4-(4-Cyclopentylbutyl)phenyl)-N-hydroxyacetamide |
| 64 | 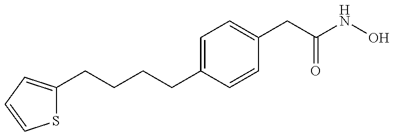 [SMILES: O=C(Cc1ccc(CCCCc2cccs2)cc1)NO] | N-Hydroxy-2-(4-(4-(thiophen-2-yl)butyl)phenyl)acetamide |
| 65 | 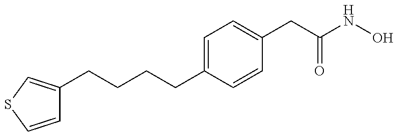 [SMILES: O=C(Cc1ccc(CCCCc2ccsc2)cc1)NO] | N-Hydroxy-2-(4-(4-(thiophen-3-yl)butyl)phenyl)acetamide |

or an N-oxide, hydrate, solvate, pharmaceutically acceptable salt, agricultural formulation, prodrug or complex thereof.

In certain embodiments of the first aspect, the compound of the invention is one of compounds 3, 4, 6, 8, 9, 10, 16, 19, 20, 29, 30, 32, 55, 56, 60, 63, 64, 65 or an N-oxide, hydrate, solvate, pharmaceutically acceptable salt, agricultural formulation, prodrug or complex thereof.

In certain embodiments of the first aspect, the compound of the invention is one of compounds 3, 4, 6, 8, 9, 10, 16, 19, 20, 29, 30, 32, 55, 56, 60 or an N-oxide, hydrate, solvate, pharmaceutically acceptable salt, agricultural formulation, prodrug or complex thereof.

### Compositions

In a second aspect, the invention provides a composition comprising an inhibitor of histone deacetylase, or an N-oxide, hydrate, solvate, pharmaceutically acceptable salt, agricultural formulation, prodrug or complex thereof, an antifungal agent, and a pharmaceutically acceptable carrier, excipient, or diluent. In one embodiment, the inhibitor is a hydroxamate-based inhibitor of histone deacetylase, more preferably a compound of Formula (I) or Formula (II). In certain embodiments the inhibitor is a prodrug of Formula (A-I) or Formula (A-II). In other embodiments, the composition comprises a selective and synergistic amount of the inhibitor of histone deacetylase, or an N-oxide, hydrate, solvate, pharmaceutically acceptable salt, agricultural formulation, prodrug or complex thereof, an antifungal effective amount of an antifungal agent, and a pharmaceutically acceptable carrier, excipient, or diluent.

In some embodiments, the antifungal agent inhibits a step in the ergosterol synthesis pathway or the synthesis of a multidrug transporter. In one embodiment, inhibiting ergosterol biosynthesis comprises inhibiting ERG₁ or ERG₁₁. In another embodiment, the multidrug transporter is CDR₁ or CDR₂.

In some embodiments, the antifungal agent is an azole selected from the group consisting of binonazole, butoconazole, clomidazole, clotrimazole, croconazole, econazole, fenticonazole, isoconazole, ketoconazole, miconazole, neticonzaole, omoconazole, oxiconazole, sertazonazole, sulconazole, tioconazole, albaconazole, fluconazole, fosfluconaole, hexaconazole, isavuconazole, itraconazole, posaconazole, ravuconazole, terconazole, voriconazole, abafungin and dimazole.

In some embodiments, the antifungal agent is selected from the group consisting of echinocandin, amphotericin B, ciclopirox, chlorophetanol, chlorphensin, filipin, flucytosine, griseofulvin, haloprogin, hamycin, natamycin, a nikkomycin, nystatin, pimaricin, polygodial, sulbentine, taurolidine, ticlatone, tolciclate, tolnaftate, undecylenic acid, amorolfin, butenafine, naftifine, terbinafine and fenpropimorph. In other embodiments, the antifungal agent is a combination of two or more antifungal agents as defined herein.

The characteristics of the pharmaceutically acceptable carrier and agricultural formulation will depend on the route of administration. Compositions of the invention may be formulated by any method well known in the pharmaceutical and agricultural arts. For pharmaceutical use, the composition may be prepared for administration by any route, including, without limitation, parenteral, oral, sublingual, transdermal, topical, intranasal, intratracheal, or intrarectal. In some embodiments, compositions of the invention are administered intravenously in a hospital setting. In certain embodiments, administration may preferably be by the oral route. For agricultural use, the compositions may be prepared as a solid or solution. In some embodiments, the solid is applied directly to the plant. In other embodiments, the solid is dissolved in a solution for spray application.

### Methods of Treating Disease

In a third aspect, the invention provides a method of selectively sensitizing a fungal cell to an antifungal agent comprising contacting the fungal cell with an antifungal effective amount of the compound or composition as described above, where the selectively sensitizing effective amount of the histone deacetylase inhibitor or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In a forth aspect, the invention provides a method of selectively enhancing the activity of an antifungal agent against a fungal cell comprising contacting the fungal cell with an antifungal effective amount of the compound or composition as described above, where the selectively enhancing effective amount of the histone deacetylase inhibitor, or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In a fifth aspect, the invention provides a method of selectively inhibiting fungal growth, comprising contacting a fungus with an antifungal effective amount of the compound or composition as described above, where the selectively inhibiting effective amount of the histone deacetylase inhibitor, or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In a sixth aspect, the invention provides a method of selectively treating a fungal infection comprising administering to an organism infected with at least one infectious fungal unit an antifungal effective amount of the compound or composition as described above, where the selectively treating effective amount of histone deacetylase inhibitor, or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In a seventh aspect, the invention provides a method of selectively reducing resistance of a fungal cell to an antifungal agent comprising contacting the fungal cell with an antifungal effective amount of the compound or composition as described above, where, the selectively reducing amount of the histone deacetylase inhibitor, or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In an eight aspect, the invention provides a method of selectively reducing antifungal agent-dependent upregulation of a gene in a fungal cell comprising contacting the fungal cell with an antifungal effective amount of the compound or composition as described above, where the selectively reducing amount of the histone deacetylase inhibitor, or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In a ninth aspect, the invention provides a method of selectively inhibiting development of an antifungal agent-resistant fungal cell upon contacting the fungal cell with an antifungal agent, comprising contacting the fungal cell with an antifungal effective amount of the compound or composition as described above, where the selectively inhibiting amount of the histone deacetylase inhibitor, or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In a tenth aspect, the invention provides a method of selectively inhibiting expression of a gene involved in ergosterol biosynthesis or a gene encoding a multidrug transporter in a fungal cell during treatment of the fungal cell with an antifungal agent, comprising contacting the fungal cell with an antifungal effective amount of the compound or composition as described above, where the selectively inhibiting amount of the histone deacetylase inhibitor, or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In an eleventh aspect, the invention provides a method of selectively promoting cidal effect of an antifungal agent on a fungal cell, comprising contacting the fungal cell with an antifungal effective amount of the compound or composition as described above, where, the selectively promoting amount of the histone deacetylase inhibitor, or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In a twelfth aspect, the invention provides a method of selectively increasing the post-antibiotic effect of an antifungal agent on a fungal cell, comprising contacting the fungal cell with an antifungal effective amount of the compound or composition as described above, where the selectively increasing effective amount of the histone deacetylase inhibitor, or an N-oxide, hydrate, solvate, pharmaceutically acceptable salt, agricultural formulation, prodrug or complex thereof, is synergistic with the antifungal effective amount of the antifungal agent. In one embodiment, the histone deacetylase inhibitor is a compound of Formula (I). In another embodiment, the compound is of Formula (II).

In one embodiment of a method according to the present invention, enhancing fungal sensitivity to the antifungal agent comprises inhibiting ergosterol biosynthesis, inhibiting a step in the ergosterol biosynthesis pathway, or inhibiting expression of a gene involved in ergosterol biosynthesis. In certain embodiments, the gene involved in ergosterol biosynthesis is selected from the group consisting of ERG₁ and ERG₁₁.

In another embodiment of a method according to the present invention, enhancing fungal sensitivity to the antifungal agent comprises inhibiting synthesis of a multidrug transporter, inhibiting expression of a gene encoding a multidrug transporter, or a part thereof. In certain embodiments, the gene involved in synthesis of a multidrug transporter is selected from the group consisting of CDR₁ and CDR₂.

In one embodiment of a method according to the present invention, the fungal cell is in or on another organism, such as, for example, a mammal or a plant.

In a certain embodiment of a method according to the present invention, a histone deacetylase inhibitor and antifungal agent, or a composition thereof, is administered to an organism. In one embodiment, the HDAC inhibitor and the antifungal agent are administered together. In another embodiment, the HDAC inhibitor and the antifungal agent are administered separately. In another embodiment, the HDAC inhibitor is administered prior to administration of the antifungal agent. In other embodiments, the HDAC inhibitor is administered after administration of the antifungal agent.

### Pharmaceutical Formulations, Dosage Forms and Agricultural Formulations

The pharmaceutical compositions described herein generally comprise a combination of a compound described herein and a pharmaceutically acceptable carrier, diluent, or excipient. Such compositions are substantially free of non-pharmaceutically acceptable components, i.e., contain amounts of non-pharmaceutically acceptable components lower than permitted by US regulatory requirements at the time of filing this application. In some embodiments of this aspect, if the compound is dissolved or suspended in water, the composition further optionally comprises an additional pharmaceutically acceptable carrier, diluent, or excipient. In other embodiments, the pharmaceutical compositions described herein are solid pharmaceutical compositions (e.g., tablet, capsules, etc.).

These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Also, pharmaceutical compositions can contain, as the active ingredient, one or more of the compounds described herein above in combination with one or more pharmaceutically acceptable carriers. In making the compositions described herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions described herein can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound described herein. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of a compound described herein.

The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as previously described. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. For example, the compounds described herein can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds described herein can also be formulated in combination with one or more additional active ingredients which can include any pharmaceutical agent such as anti-viral agents, vaccines, antibodies, immune enhancers, immune suppressants, anti-inflammatory agents and the like.

Agricultural formulationsmay be prepared as a solid or solution. In some embodiments, the solid is a granule, microgranule or a dust. In other embodiments, the composition is prepared as a powder to be dissolved in a solution, optionally containing an additive or adjuvant, for spray application. Commonly used additives or adjuvants, include, but are not limited to surfacants, non-ionic surfacants, emulsifiers, wetting agents, diluents, and spreader-stickers.

Synthetic Schemes and Experimental Procedures

Some examples of the compounds according to the first aspect of the invention are given below. These examples merely serve to exemplify some of the compounds of the invention and do not limit the scope of the invention.

The compounds of the invention can be prepared according to the reaction schemes or the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

All reagents and solvents were obtained from commercial sources and used as received. ¹H-NMR spectra were recorded on a Mercury Plus Varian 400 MHz instrument in the solvents indicated. Low resolution mass-spectra (LRMS) were acquired on an Agilent MSD instrument. Analytical HPLC was performed on an Agilent 1100 instrument using Zorbax 3 μm, XDB-C8, 2.1×50 mm column; eluting with methanol/water containing 0.1% formic acid, with a gradient 5-95% methanol in 15 minutes. Automated column chromatography was performed on a Biotage SP1 or Biotage SP4 instruments using Biotage® SNAP, SiliaSep™ or SiliaFlash® cartridges. Flash column chromatography was performed using silica gel (40-63 μM, pore size 60 Å, SiliCycle®).

The following abbreviations and/or acronyms are used within the examples:

| | |
|---|---|
| AcOEt | ethyl acetate |
| AcOH | acetic acid |
| aq | aqueous |
| bd | broad doublet (NMR) |
| CV | column volume |
| d | doublet (NMR) |
| dd | doublet of doublets |
| | (NMR) |
| DAST | Diethylaminosulfur |
| | trifluoride |
| DCM | dichloromethane |
| DIPEA | diisopropyl ethylamine |
| DMF | N,N-dimethylformamide |
| DMSO | dimethylsulfoxide |
| DMSO-d₆ | dimethylsulfoxide-d₆ |
| EDC | 1-(3-dimethyl |
| | aminopropyl)-3-ethyl- |
| | carbodiimide |
| Et₃N | triethylamine |
| EtOH | ethanol |
| EtOAc | ethyl acetate |
| Et₂O | diethyl ether |
| equiv | equivalent |
| g | gram (grams) |
| hr (hrs) | hour (hours) |
| HOBt | 1-hydroxybenzotriazole |
| m | multiplet (NMR) |
| mL | milliliter |
| μl | microliter |
| MeOH | methanol |
| MeOH-d₄ | methanol-d₄ |
| mg | milligram (milligrams) |
| min | minute (minutes) |
| MS | mass-spectroscopy |
| m/z | mass-to-charge ratio |

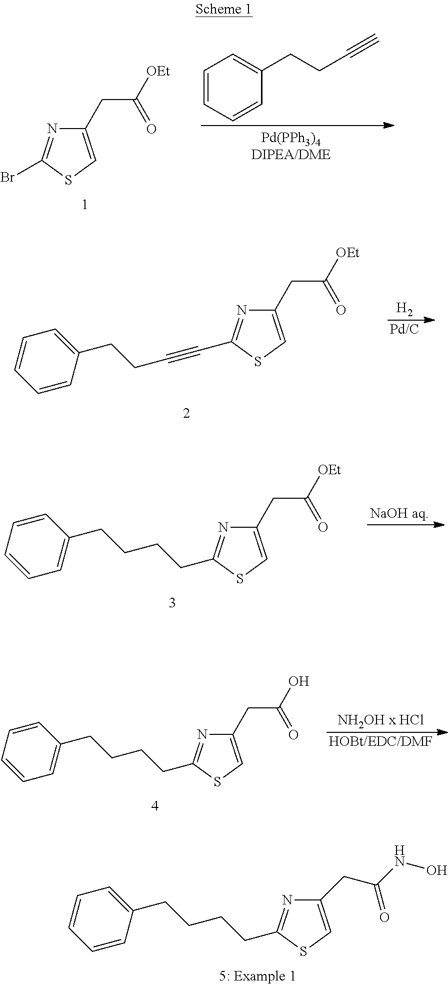

### Example 1

### N-Hydroxy-2-(2-(4-phenylbutyl)thiazol-4-yl)acetamide (5, Example 1)

### Step 1. Ethyl 2-(2-(4-phenylbut-1-ynyl)thiazol-4-yl)acetate (2)

To a degassed solution of the ethyl 2-(2-bromothiazol-4-yl)acetate (1, 0.645 g, 2.58 mmol) (obtained according to the procedure similar to the one described in WO 2006/114274 A1), Pd(PPh₃)₄ (149 mg, 0.129 mmol) and CuI (74 mg, 0.387 mmol) in DME (60 mL) was bubbled nitrogen (˜15 min). The solution was preheated to 75° C. and to the hot solution were added the DIPEA (1.35 mL, 7.74 mmol) and the phenylbutyne (0.45 mL, 3.20 mmol)). The reaction mixture was stirred for 4 hrs. Another portion of the alkyne (0.2 mL, 1.42 mmol) was added and the reaction mixture was stirred at the same conditions for an additional 20 hrs. The reaction mixture was then cooled to rt, evaporated and the residue was subjected to flash column chromatography, eluent EtOAc-Hexanes (1:4) to afford the title compound 2 (0.482 g, 62.4% yield) as an oil. MS (m/z): 300.2 (M+H).

### Step 2. Ethyl 2-(2-(4-phenylbutyl)thiazol-4-yl)acetate (3)

To a solution of the alkyne 2 (480 mg, 1.603 mmol) in EtOH (40 mL) was added Pd/C, 10%—Degussa type (50 mg). The air from reaction flask was evacuated and the contents of the flask were stirred under the atmosphere of hydrogen for 24 hrs. Two more portions of the Pd/C—50 mg each, were added to the reaction mixture after 4 and 8 hrs of the reaction course. The mixture was filtered through a celite pad and evaporated to afford the title compound 3 (480 mg, 99% yield) as an oil. MS (m/z): 304.1 (M+H).

### Step 3. 2-(2-(4-Phenylbutyl)thiazol-4-yl)acetic acid (4)

To a solution of the ester 3 (480 mg, 1.582 mmol) in THF (10 mL) was added a 3N solution of NaOH (1.582 mL). The reaction mixture was vigorously stirred for 7.5 hrs at rt. The reaction mixture was then acidified by adding 1N HCl solution to pH 7 and extracted with EtOAc. The extract was washed with water, dried over anhydrous MgSO₄, filtered and evaporated. The residue was purified via a flash column chromatography (eluent EtOAc) to afford the title compound 4 (339 mg, 78% yield) as an oil that has solidified upon standing at rt. MS (m/z): 276.1 (M+H).

### Step 4. N-Hydroxy-2-(2-(4-phenylbutyl)thiazol-4-yl)acetamide (5, Example 1)

To a stirred solution of the acid (335 mg, 1.217 mmol) in DMF (10 mL) were added HOBt×H₂O (224 mg, 1.46 mmol) and EDC x HCl (303 mg, 1.582 mmol). The reaction mixture was stirred for 1 hr at rt. Then NH₂OH x HCl (423 mg, 6.08 mmol) and Et₃N (1.273 mL, 9.12 mmol) were added and the combined mixture was stirred at rt for 20 hrs. DMF was partially evaporated; the residue was diluted with water and extracted with EtOAc. The extract was collected, dried over MgSO₄, filtered and evaporated. The residue was purified by column chromatography, eluent MeOH (10%) in DCM to afford the title compound 5 (17 mg, 4.8% yield). ¹H NMR (400 MHz, DMSO-d₆) δ (ppm): 10.60 (bs, 1H), 8.34 (bs, 1H), 7.29-7.25 (m, 2H), 7.20-7.15 (m, 4H), 3.39 (s, 2H), 2.94 (t, J=6.8 Hz, 2H), 2.61 (t, J=7.2 Hz, 2H), 1.69-1.64 (m, 4H). MS (m/z): 291.2 (M+H).

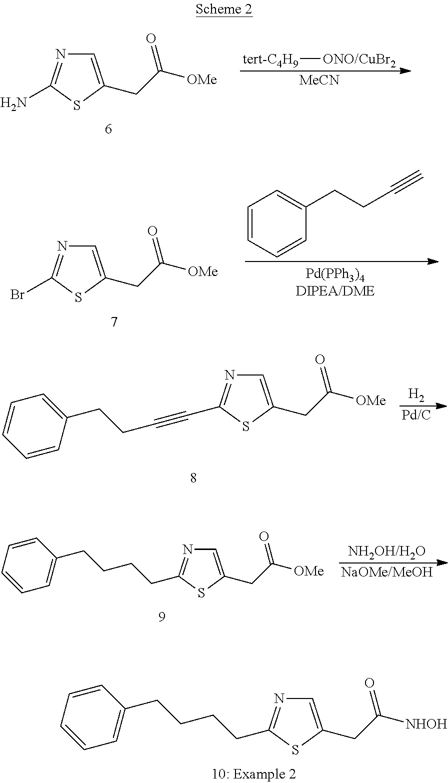

### Example 2

### N-Hydroxy-2-(2-(4-phenylbutyl)thiazol-5-yl)acetamide (10, Example 2)

### Step 1. Methyl 2-(2-bromothiazol-5-yl)acetate (7)

Title compound 7 was synthesized from the commercially available methyl 2-(2-aminothiazol-5-yl)acetate (6) by following a procedure similar to the one disclosed in WO 2006/114274 A1, in 39.8% yield. MS (m/z): 236.0 and 238.0 (M+H).

### Step 2. Methyl 2-(2-(4-phenylbut-1-ynyl)thiazol-5-yl)acetate (8)

Title compound 8 was synthesized from the bromothiazole 7 in 66.2% yield by following the procedure described above for the synthesis of compound 2 (Scheme 1). MS (m/z): 286.0 (M+H).

### Step 3. Methyl 2-(2-(4-phenylbutyl)thiazol-5-yl)acetate (9)

To a solution of the alkyne 8 (300 mg, 1.051 mmol) in a solvent mixture EtOH (40 mL) and AcOH (1 mL) was added Pd/C, 10%—Degussa type (112 mg,). The air from the reaction flask was evacuated and the contents of the flask were stirred under the atmosphere of hydrogen for 24 hrs. The reaction mixture was filtered through a celite pad, evaporated, re-dissolved in AcOEt, washed with a saturated NaHCO₃ solution, dried over anhydrous MgSO₄, filtered, evaporated then dried in vacuum. The oily yellow product was purified by a flash column chromatography, eluent EtOAc-Hexanes (1:4) to afford title compound 9 (128 mg, 42.1% yield) as a colorless oil. MS (m/z): 290.1 (M+H).

### Step 4. N-Hydroxy-2-(2-(4-phenylbutyl)thiazol-5-yl)acetamide (10, Example 2)

To a solution of the ester 9 (125 mg, 0.432 mmol) in MeOH (7.5 mL) at 0° C. was added a 25% wt/wt solution of MeONa (0.495 mL, d=0.945 g/mL, 2.16 mmol) followed by a 50% aqueous solution of hydroxylamine (0.265 mL, d=1.078 g/mL, 4.32 mmol). The reaction mixture was stirred at the same temperature for 2 hrs. The reaction mixture was treated with 1N HCl (pH 7-8), partially evaporated, diluted with brine and extracted with EtOAc. The extract was collected, dried over MgSO₄, filtered and evaporated. The residue was purified twice by flash column chromatography using 10% MeOH in DCM as the first eluent, and 5% MeOH in EtOAc as the second eluent. The isolated material was lyophilized to afford the title compound 10 (26 mg, 20.7% yield) as a white fluffy material. ¹H NMR (400 MHz, MeOH-d₄) δ (ppm): 7.43 (s, 1H), 7.25-7.22 (m, 2H), 7.16-7.13 (m, 3H), 3.60 (s, 2H), 2.98 (t, J=7.0 Hz, 2H), 2.63 (t, J=7.4 Hz, 2H), 1.79-1.66 (m, 4H). MS (m/z): 291.0 (M+H).

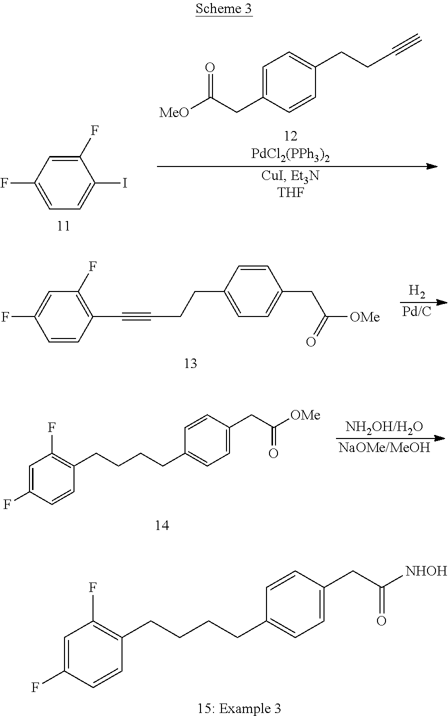

### Example 3

### 2-(4-(4-(2,4-Difluorophenyl)butyl)phenyl)-N-hydroxyacetamide (15, Example 3)

### Step 1. Methyl 2-(4-(4-(2,4-difluorophenyl)but-3-ynyl)phenyl)acetate (13)

To a degassed solution of the iodide 11 (700 mg, 2.92 mmol), methyl 2-(4-(but-3-ynyl)phenyl)acetate (12) (190 mg, 0.939 mmol, WO 2008/074132 A1) and TEA (0.39 mL, d=0.7255 g/mL, 2.82 mmol) in THF (10 mL) were added PdCl₂(PPh₃)₂ (33 mg, 0.047 mmol) and CuI (18 mg, 0.094 mmol). The reaction mixture was stirred for 18 hours at rt. The mixture was evaporated, re-dissolved in DCM, washed with 1N HCl then brine. The organic phase was dried over anhydrous MgSO₄, filtered and evaporated. The residue was purified by flash column chromatography, eluent 20% EtOAc in hexanes to afford the title compound 13 (106 mg, 35.9% yield) as an oily material. ¹H NMR (400 MHz, CDCl₃) δ (ppm): 7.36-7.29 (m, 1H), 7.23 (b s, 4H), 6.81 (t, J=8.4 Hz, 2H), 3.69 (s, 3H), 3.61 (s, 2H), 2.92 (t, J=7.4 Hz, 2H), 2.71 (t, J=7.6 Hz, 2H);

### Step 2. Methyl 2-(4-(4-(2,4-difluorophenyl)butyl)phenyl)acetate (14)

To a solution of the alkyne 13 (106 mg, 0.337 mmol) in MeOH (7 mL) was added Pd/C, 10%—Degussa type (50 mg). The air from reaction flask was evacuated and the contents of the flask were stirred under the atmosphere of hydrogen for 24 hrs. The mixture was filtered through a celite pad and evaporated to afford the title compound 3 (99 mg, 92% yield) as an oil. ¹H NMR (400 MHz, CDCl₃) δ (ppm): 7.20 (d, J=8.2 Hz, 2H), 7.13-7.07 (m, 3H), 6.80-6.73 (m, 2H), 3.69 (s, 3H), 3.59 (s, 2H), 2.61 (t, J=7.0 Hz, 4H), 1.65-1.56 (m, 4H).

### Step 3. 2-(4-(4-(2,4-Difluorophenyl)butyl)phenyl)-N-hydroxyacetamide (15, Example 3)

To a solution of the ester 14 (99 mg, 0.311 mmol) in MeOH (8.0 mL) at 0° C. was added a 25% wt/wt solution of MeONa (0.356 mL, d=0.945 g/mL, 1.555 mmol) followed by a 50% aqueous solution of hydroxylamine (0.190 mL, d=1.078 g/mL, 3.11 mmol). The reaction mixture was stirred at the same temperature for 2 hrs. The reaction mixture was then treated with 1N HCl (pH 7-8), partially evaporated, diluted with brine to form a precipitate that was collected by filtration, washed with water and dried to afford the title compound 15 (88 mg, 89% yield). ¹H NMR (400 MHz, DMSO-d₆) δ (ppm): 10.61 (bs, 1H), 8.79 (bs, 1H), 7.34-7.28 (m, 2H), 7.17-7.08 (m, 5H), 7.01-6.99 (m, 1H), 3.21 (s, 2H), 2.61-2.54 (m, 4H), 1.54 (t, J=3.5 Hz, 4H). MS (m/z): 320.0 (M+H).

Compounds 16-18 (examples 4-6) were prepared in three steps using the procedures similar to the ones described above for the synthesis of compound 15 (Scheme 3) starting from commercially available 4-iodobenzaldehyde and 4-iodobiphenyl, or known 5-iodo-1-methyl-1H-indole (WO 2008/070908 A1), respectively.

TABLE 5
Characterization of compounds 16-18 (examples 4-6).

| Cpd | Ex. | Structure | Characterization |
|---|---|---|---|
| 16 | 4 | 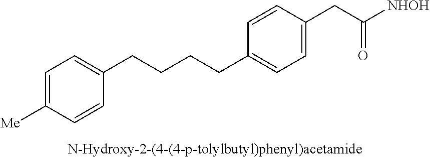 | ¹H NMR (400 MHz, DMSO-d₆) δ (ppm): 10.61 (bs, 1H), 8.79 (bs, 1H), 7.15-7.03 (m, 8H), 3.21 (s, 2H), 2.55-2.53 (m, 4H), 2.24 (s, 3H), 1.55-1.51 (m, 4H). MS (m/z): 298.2 (M + H). |
| 17 | 5 | 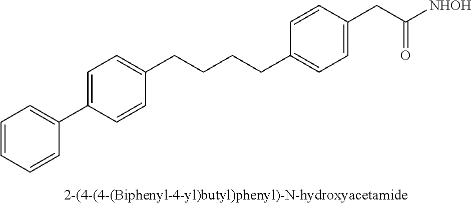 | ¹H NMR (400 MHz, DMSO-d₆) δ (ppm): 10.63 (bs, 1H), 8.81 (bs, 1H), 7.64-7.61 (m, 2H), 7.56 (dd, J = 1.8 and 6.3 Hz, 2H), 7.44 (t, J = 7.4 Hz, 2H), 7.35-7.31 (m, 1H), 7.26 (d, J = 8.0 Hz, 2H), 7.15 (d, J = 8.2 Hz, 2H), 7.11 (d, J = 8.2 Hz, 2H), 3.22 (s, 2H), 2.63 (t, J = 6.8 Hz, 2H), 2.58 (t, J = 7.0 Hz, 2H), 1.59 (t, J = 3.5 Hz, 4H). MS (m/z): 360.1 (M + H) |
| 18 | 6 | 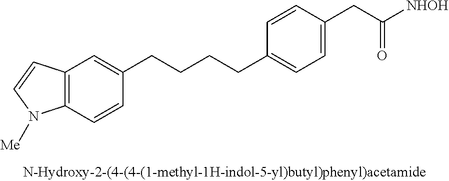 | ¹H NMR (400 MHz, DMSO-d₆) δ (ppm): signals of NH and OH protons are not seen; 7.31-7.29 (m, 2H), 7.24 (d, J = 3.1 Hz, 1H), 7.12 (d, J = 8.2 Hz, 2H), 7.06 (d, J = 8.2 Hz, 2H), 6.96 (dd, J = 1.4 and 8.4 Hz; 1H), 6.31 (dd, J = 0.8 and 3.1 Hz; 1H), 3.74 (s, 3H), 3.16 (s, 2H), 2.65 (t, J = 7.0 Hz, 2H); 2.55 (t, J = 7.3 Hz, 2H); 1.59-1.55 (m, 4H). MS (m/z): 337.2 (M + H). |

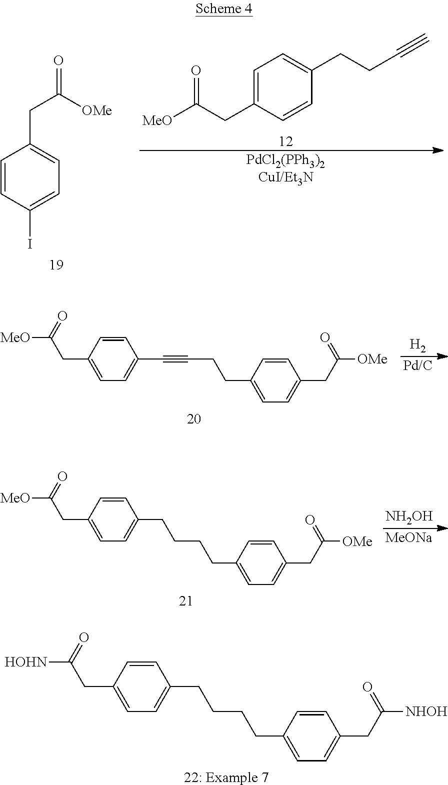

### Example 7

### 2,2′-(4,4′-(Butane-1,4-diyl)bis(4,1-phenylene))bis(N-hydroxyacetamide) (22, Example 7) Step 1. Dimethyl 2,2′-(4,4′-(but-1-yne-1,4-diyl)bis(4,1-phenylene))diacetate (20)

To a degassed solution of the iodide 19 (358 mg, 1.298 mmol, WO 2008/074132), acetylene 12 (250 mg, 1.236 mmol, Scheme 3) and TEA (0.517 mL, d=0.7255 g/L, 3.0 mmol) were added PdCl₂(PPh₃)₂ (43 mg, 0.062 mmol) and CuI (24 mg, 0124 mmol). The reaction mixture was stirred for 18 hours at rt. The mixture was evaporated, re-dissolved in DCM, washed with 1N HCl then brine. The organic phase was dried over anhydrous MgSO₄, filtered and evaporated. The residue was purified by flash column chromatography, eluent DCM to afford the title compound 20 (315 mg, 72.7% yield) as an oil. ¹H NMR (400 MHz, CDCl₃) δ (ppm): 7.32 (d, J=8.2 Hz, 2H), 7.23 (bs, 4H), 7.19 (d, J=8.4 Hz, 2H), 3.689 (s, 3H), 3.687 (s, 3H), 3.609 (s, 2H), 3.603 (s, 2H), 2.90 (t, J=7.6 Hz, 2H), 2.67 (t, J=7.4 Hz, 2H).

### Step 2. Dimethyl 2,2′-(4,4′-(butane-1,4-diyl)bis(4,1-phenylene))diacetate (21)

A solution of 20 (310 mg, 0.885 mmol) in MeOH (8 mL) was subjected to hydrogenation at 1 atm pressure over 96 hours. The reaction mixture was then filtered through a celite pad; the pad was washed with acetone and the filtrate and washings were combined and evaporated to afford the title compound 21 (310 mg, 99%) as a white solid which was used in the next step with no additional purification. ¹H NMR (400 MHz, CDCl₃) δ (ppm): 7.18 (d, J=8.2 Hz, 4H), 7.12 (d, J=8.2 Hz, 4H), 3.68 (s, 6H), 3.59 (s, 4H), 2.60 (b s, 4H), 1.66-1.62 (m, 4H).

### Step 3. 2,2′-(4,4′-(Butane-1,4-diyl)bis(4,1-phenylene))bis(N-hydroxyacetamide) (22, Example 7)

A suspension of 21 in MeOH (10 mL) was treated sequentially with 50% aqueous hydroxylamine solution (1.089 mL, d=1.078 g/mL) and 25% wt/wt NaOMe solution in MeOH (2.032 mL, d=0.945 g/mL) at 0° C. The suspension gradually turned into a solution and after ca 60 min a new precipitate was formed. The reaction mixture was stirred altogether for 2 hours, acidified with 1N HCl (pH 6-7), treated with brine and stirred overnight. The white precipitate was collected by filtration, washed with water and dried to afford the title compound 22 (314 mg, 99% yield). ¹H NMR (400 MHz, DMSO-d₆) δ (ppm): 10.62 (bs, 1H), 8.81 (bs, 1H), 7.14 (d, J=8.4 Hz, 2H), 7.09 (d, J=8.4 Hz, 2H), 3.21 (s, 2H), 2.55 (bs, 4H), 1.53 (bs, 4H).

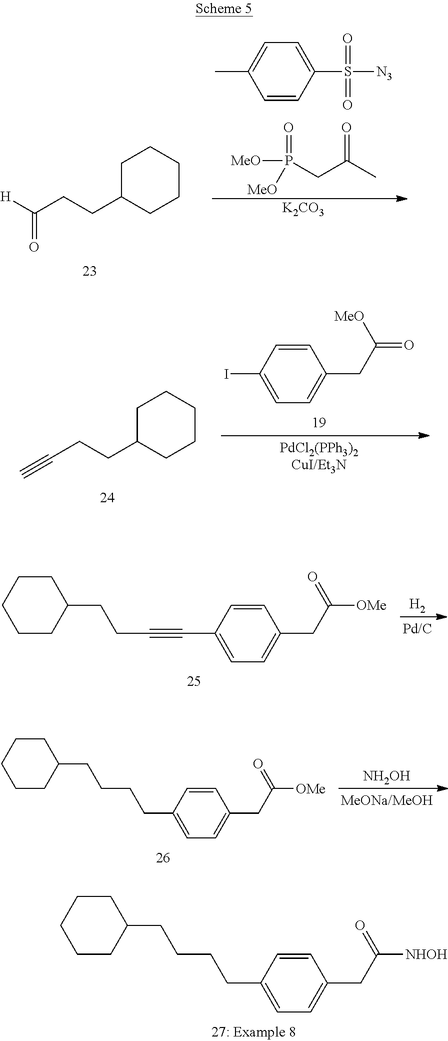

### Example 8

### 2-(4-(4-Cyclohexylbutyl)phenyl)-N-hydroxyacetamide (27, Example 8)

### Step 1. But-3-ynylcyclohexane (24)

To a suspension of K₂CO₃ (6.2 g, 44.86 mmol) in MeCN (76 mL) was added a solution of the 4-methylbenzenesulfonyl azide (3.6 g, 18.26 mmol) in MeCN (12 mL) followed by addition of the dimethyl 2-oxopropylphosphonate (3.0 g, 18.06 mmol) in MeCN (12 mL) (B. Liepold, et al. Synthesis, 2004, 1, 59-62). The reaction mixture was stirred at rt for 2 hours. A solution of the aldehyde 23 (2.0 g, 14.26 mmol) in MeOH (24 mL) was then added and the reaction mixture was stirred at rt overnight. The reaction mixture was filtered; the filtrate was collected, evaporated and partitioned between water and EtOAc. The organic phase was dried over anhydrous MgSO₄, filtered and evaporated. The residue was suspended in a mixture EtOAc/hexanes, the white precipitate was discarded and the filtrate was collected and evaporated. The remained material was purified by flash column chromatography, eluent 10% EtOAc in hexanes to afford the title compound 24 as a light colorless oil (0.32 g, 16.5% yield). ¹H NMR (400 MHz, CDCl₃) δ (ppm): 2.22-2.17 (m, 2H), 1.93 (t, J=2.5 Hz, 1H), 1.73-1.62 (m, 5H), 1.46-1.11 (m, 6H), 0.92-0.82 (m, 2H).

### Steps 2-4. 2-(4-(4-Cyclohexylbutyl)phenyl)-N-hydroxyacetamide (27, Example 8)

Title compound 27 was obtained starting from the alkyne 24 by following the procedures similar to the ones described above for the synthesis compound 15 (example 3, Scheme 3) via intermediates methyl 2-(4-(4-cyclohexylbut-1-ynyl)phenyl)acetate (25) [colorless oil, 64.4% yield; ¹H NMR (400 MHz, CDCl₃) δ (ppm): 7.34 (d, J=8.0 Hz, 2H), 7.19 (d, J=8.0 Hz, 2H), 3.69 (s, 3H), 3.60 (s, 2H), 2.40 (t, J=7.4 Hz, 2H), 1.76-1.64 (m, 4H), 1.48 (doublet of triplets, J=7.2 Hz, 2H), 1.43-1.14 (m, 4H), 0.96-0.87 (m, 3H)] and methyl 2-(4-(4-cyclohexylbutyl)phenyl)acetate (26) [colorless oil, 61.9% yield; ¹H NMR (400 MHz, CDCl₃) δ (ppm): 7.26-7.12 (m, 4H), 3.69 (s, 3H), 3.59 (s, 2H), 2.58 (t, J=7.6 Hz, 2H), 1.69-1.55 (m, 7H), 1.34-1.29 (m, 2H), 1.22-1.17 (m, 4H), 0.85 (b quartet, 2H)]. The compound 27 was isolated as a white solid in 98% yield. ¹H NMR (400 MHz, DMSO-d₆) δ (ppm): 10.62 (bs, 1H), 8.80 (bs, 1H), 7.14 (d, J=8.0 Hz, 2H), 7.09 (d, J=8.0 Hz, 2H), 3.23 (s, 2H), 2.52 (t, J=7.6 Hz, 2H), 1.66-1.47 (m, 7H), 1.30-1.08 (m, 8H), 0.86-0.81 (m, 2H). MS (m/z): 290.1 (M+H).

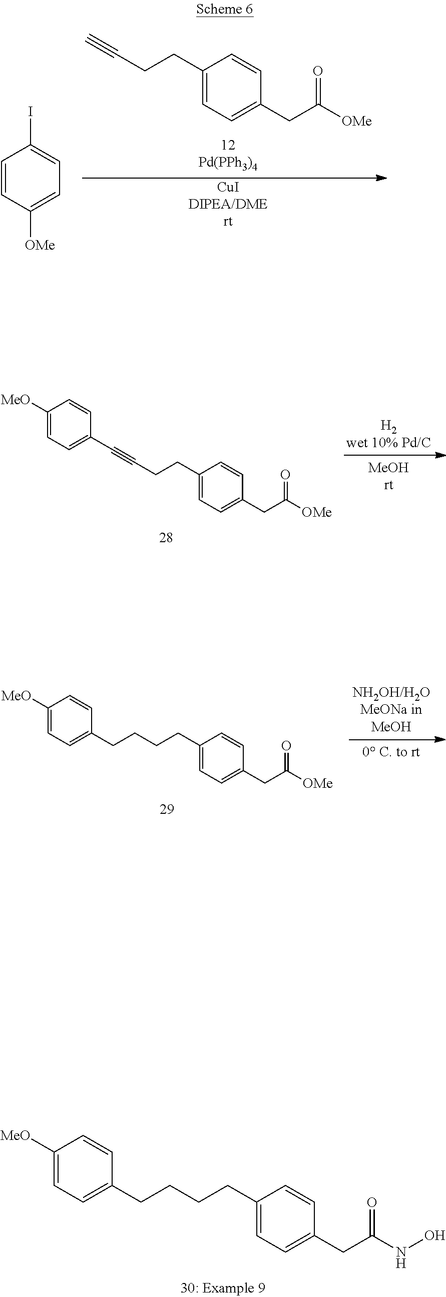 [SMILES: COc1ccc(I)cc1 ; C#CCCc1ccc(CC(=O)OC)cc1 ; "CuIDIPEA/DMErt" ; COC(=O)Cc1ccc(CCC#Cc2ccc(OC)cc2)cc1 ; COC(=O)Cc1ccc(CCCCc2ccc(OC)cc2)cc1 ; COc1ccc(CCCCc2ccc(CC(=O)NO)cc2)cc1]

### Example 9

### N-Hydroxy-2-(4-(4-(4-methoxyphenyl)butyl)phenyl)acetamide (30, Example 9)

### Step 1. Methyl 2-(4-(4-(4-methoxyphenyl)but-3-ynyl)phenyl)acetate (28)

To a degassed solution of 4-iodoanisole (555 mg, 2.37 mmol), Pd(PPh₃)₄ (114 mg, 0.10 mmol), CuI (56.5 mg, 0.30 mmol) and DIPEA (1.04 mL, 5.93 mmol) in DME (25 mL) was added methyl 2-(4-(but-3-ynyl)phenyl)acetate (12) (400 mg, 1.98 mmol, WO 2008/074132 A1). The reaction mixture was stirred at rt for 18 h, concentrated, diluted with ethyl acetate and successively washed with 0.2N HCl and brine, dried over anhydrous sodium sulfate, filtered and concentrated. The residue was purified by Biotage (Snap 50 g cartridge; AcOEt/hexanes: 0/100 to 20/80 over 15 CV), to afford the title compound 28 (484 mg, 1.57 mmol, 79% yield) as a yellow oil. MS (m/z): 331.4 (M+Na).

### Step 2. Methyl 2-(4-(4-(4-methoxyphenyl)butyl)phenyl)acetate (29)

To a solution of compound 28 (484 mg, 1.57 mmol) in MeOH (25 mL) was added wet 10% Pd/C Degussa type 101 (33 mg, 0.16 mmol). The suspension was stirred under H₂ atmosphere at rt for 17 h, filtered through a celite pad, washed with MeOH and concentrated to afford the title compound 29 (colorless oil, 439 mg, 1.41 mmol, 90% yield) that was used in the next step without any further purification. MS (m/z): 335.4 (M+Na).

### Step 3. N-Hydroxy-2-(4-(4-(4-methoxyphenyl)butyl)phenyl)acetamide (30, Example 9)

To a stirred solution of compound 29 (0.439 g, 1.41 mmol) and 50% aqueous solution of hydroxylamine (0.86 mL, 14.05 mmol) in MeOH (20 mL) at 0° C. was added a solution of 25% wt/wt solution of sodium methoxide in methanol (1.61 mL, 7.03 mmol). After 45 min stirring at 0° C., the reaction mixture was allowed to warm-up to rt over 30 min. The reaction mixture was then concentrated, diluted with water, and the pH was adjusted to 7-8 with 1N HCl to form a precipitate that was collected by filtration, rinsed with water and dried to afford the title compound 30 (321 mg, 1.02 mmol, 73% yield) as a white solid. ¹H NMR (400 MHz, DMSO-d₆) δ (ppm): 10.58 (bs, 1H), 8.75 (bs, 1H), 7.20-7.00 (m, 6H), 6.82 (d, J=8.6 Hz, 2H), 3.71 (s, 3H), 3.22 (s, 2H), 2.59-2.50 (m, 4H), 1.59-1.47 (m, 4H). MS (m/z): 314.4 (M+H) and 336.4 (M+Na).

Compounds 31-35 (examples 10-14) were prepared in three steps by coupling the functionalyzed iodonoarene (commercially available or described in Scheme 7) with methyl 2-(4-(but-3-ynyl)phenyl)acetate (12) similarly to compound 30 (Scheme 6). All the final compounds were purified by normal phase and/or C18 reverse phase preparative chromatography on Biotage.

TABLE 6
Characterization of compounds 31-35 (examples 10-14).

| Cpd | Ex. | Structure | Characterization |
|---|---|---|---|
| 31 | 10 | 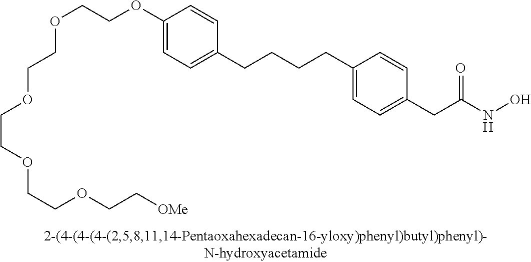 [SMILES: COCCOCCOCCOCCOCCOc1ccc(CCCCc2ccc(CC(=O)NO)cc2)cc1] | ¹H NMR (400 MHz, DMSO-d₆) δ (ppm): 10.60 (bs, 1H), 8.78 (bs, 1H), 7.20-7.00 (m, 6H), 6.82 (d, J = 8.6 Hz, 2H), 4.06-4.00 (m, 2H), 3.74-3.69 (m, 2H), 3.60-3.46 (m, 14H), 3.44- 3.39 (m, 2H), 3.31 (s, 3H), 3.23 (s, 2H), 2.59-2.49 (m, 4H, partially overlapped by the residual DMSO signal), 1.60-1.46 (m, 4H). MS (m/z): 534.3 (M + H) |
| 32 | 11 | 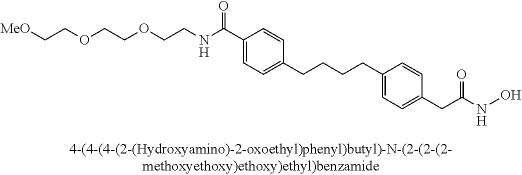 [SMILES: COCCOCCOCCNC(=O)c1ccc(CCCCc2ccc(CC(=O)NO)cc2)cc1] | ¹H NMR (400 MHz, DMSO-d₆) δ (ppm): 10.61 (bs, 1H), 8.79 (s, 1H), 8.41 (t, J = 5.6 Hz, 1H), AB system (δ_{A} = 7.74, δ_{B} = 7.25, J_{AB} = 8.2 Hz, 4H), A′B′ system (δ_{A′} = 7.14, δ_{B′} = 7.09, J_{A′B′} = 8.0 Hz, 4H), 3.55-3.47 (m, 8H), 3.42-3.36 (m, 4H), 3.23- 3.19 (m, 5H), 2.64 (t, J = 7.0 Hz, 2H), 2.56 (t, J = 6.9 Hz, 2H), 1.64-1.48 (m, 4H). MS (m/z): 473.4 (M + H). |
| 33 | 12 | 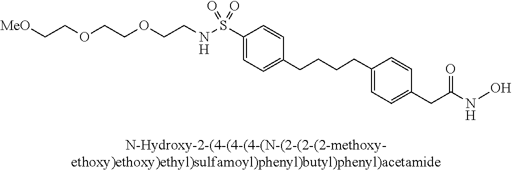 [SMILES: COCCOCCOCCNS(=O)(=O)c1ccc(CCCCc2ccc(CC(=O)NO)cc2)cc1] | ¹H NMR (400 MHz, DMSO-d₆) δ (ppm): 10.62 (bs, 1H), 8.80 (bs, 1H), AB system (δ_{A} = 7.69, δ_{B} = 7.39, J_{AB} = 8.4 Hz, 4H), 7.61 (t, J = 5.6 Hz, 1H), A′B′ system (δ_{A′} = 7.14, δ_{B′} = 7.09, J_{A′B′} = 8.1 Hz, 4H), 3.50-3.33 (m, 10H), 3.25-3.18 (m, 5H), 2.87 (q, J = 5.7 Hz, 2H), 2.67 (t, J = 7.1 Hz, 2H), 2.56 (t, J = 6.8 Hz, 2H), 1.66- 1.49 (m, 4H). MS (m/z): 509.3 (M + H). |
| 34 | 13 | 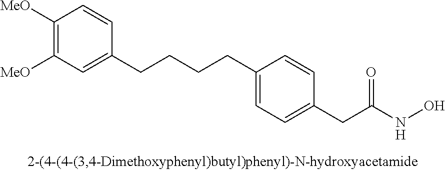 [SMILES: COc1ccc(CCCCc2ccc(CC(=O)NO)cc2)cc1OC] | ¹H NMR (400 MHz, DMSO-d₆) δ (ppm): 10.62 (bs, 1H), 8.80 (bs, 1H), AB system (δ_{A} = 7.14, δ_{B} = 7.09, J_{AB} = 8.1 Hz, 4H), ABX system (δ_{A} = 6.82, δ_{B} = 6.66, δ_{X} = 6.76, J_{AB} = 8.0 Hz, J_{BX} = 2.0 Hz, J_{AX} = 0 Hz, 3H), 3.71 and 3.69 (2s, 6H), 3.21 (s, 2H), 2.60-2.51 (m, 4H), 1.62-1.48 (m, 4H). MS (m/z): 344.1 (M + H). |
| 35 | 14 | 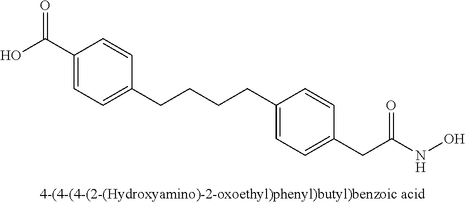 [SMILES: O=C(Cc1ccc(CCCCc2ccc(C(=O)O)cc2)cc1)NO] | ¹H NMR (400 MHz, DMSO-d₆) δ (ppm): 10.64 (bs, 1H), 2H are missing, AB system (δ_{A} = 7.83, δ_{B} = 7.26, J_{AB} = 8.2 Hz, 4H), A′B′ system (δ_{A′} = 7.14, δ_{B′} = 7.09, J_{A′B′} = 8.1 Hz, 4H), 3.21 (s, 2H), 2.65 (t, J = 6.8 Hz, 2H), 2.56 (t, J = 6.8 Hz, 2H), 1.66-1.46 (m, 4H). MS (m/z): 328.1 (M + H). |

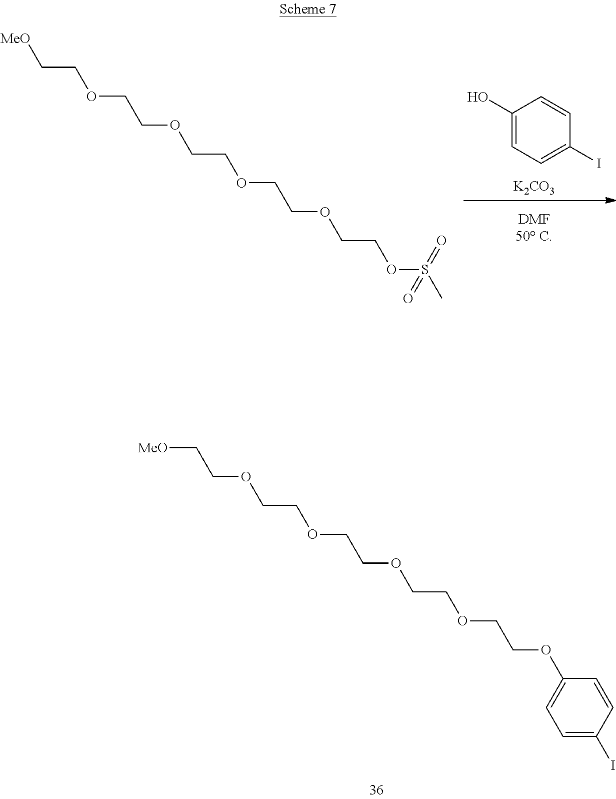 [SMILES: COCCOCCOCCOCCOCCOS(C)(=O)=O ; "K2CO3" ; Oc1ccc(I)cc1 ; COCCOCCOCCOCCOCCOc1ccc(I)cc1]

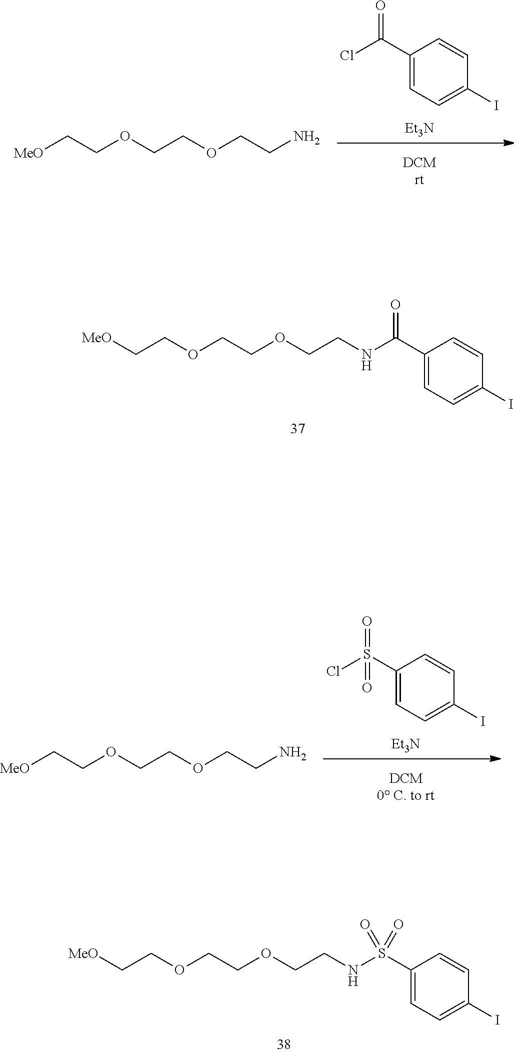

### 16-(4-Iodophenoxy)-2,5,8,11,14-pentaoxahexadecane (36)

To a stirred solution of 4-iodophenol (1.50 g, 6.82 mmol) and 2,5,8,11,14-pentaoxahexadecan-16-yl methanesulfonate (2.70 g, 8.18 mmol) in DMF (30 ml) under nitrogen at rt was added potassium carbonate (2.356 g, 17.04 mmol). The reaction mixture was heated at 50° C. overnight, cooled-down to rt, diluted with AcOEt, washed with water and brine, dried over anhydrous MgSO₄, filtered and concentrated. The residue was purified by Biotage (Snap 25 g cartridge; AcOEt/hexanes: 50/50 to 100/0 over 30 CV, 254 nm for the wavelength collection), to afford the desired product 36 (2.82 g, 6.21 mmol, 91% yield) as a pale yellow oil. MS (m/z): 477.1 (M+Na).

### 4-Iodo-N-(2-(2-(2-methoxyethoxy)ethoxy)ethyl)benzamide (37)

To a stirred solution of 4-iodobenzoyl chloride (200 mg, 0.75 mmol) in DCM (15 ml) under nitrogen at 0° C. were added slowly triethylamine (314 μl, 2.25 mmol) and a solution of 2-(2-(2-methoxyethoxy)ethoxy)ethanamine (99 mg, 0.976 mmol) in DCM (2 mL). The reaction mixture was stirred at rt overnight, quenched with MeOH, concentrated, diluted with AcOEt, and successively washed with a saturated aqueous solution of sodium bicarbonate, water and brine, dried over anhydrous MgSO₄, filtered and concentrated. The residue was purified by Biotage (Snap 25 g cartridge; MeOH/DCM: 0/100 to 5/95 over 20 CV) to afford the desired product 37 (207 mg, 0.53 mmol, 70% yield) as a colorless oil. MS (m/z): 393.98 [M+H] and 415.99 [M+Na]. The material was used in the next step with no additional purification.

### 4-Iodo-N-(2-(2-(2-methoxyethoxy)ethoxy)ethyl)benzenesulfonamide (38)

To a stirred solution of 4-iodobenzenesulfonyl chloride (300 mg, 0.99 mmol) in DCM (15 ml) under nitrogen at 0° C. were added slowly triethylamine (415 μl, 2.98 mmol) and a solution of 2-(2-(2-methoxyethoxy)ethoxy)ethanamine (151 mg, 1.49 mmol) in DCM (3 ml). The reaction mixture was stirred at rt overnight, quenched with MeOH, concentrated, diluted with AcOEt, and successively washed with a saturated aqueous solution of sodium bicarbonate, a saturated aqueous solution of ammonium chloride, water and brine, dried over anhydrous MgSO₄, filtered and concentrated. The residue was purified twice by Biotage (Snap 25 g cartridge; MeOH/DCM: 0/100 to 3/97 over 30 CV) to afford the desired product 38 (275 mg, 0.64 mmol, 64% yield) as a colorless sticky oil. MS (m/z): 430.02 [M+H] and 452.01 [M+Na].

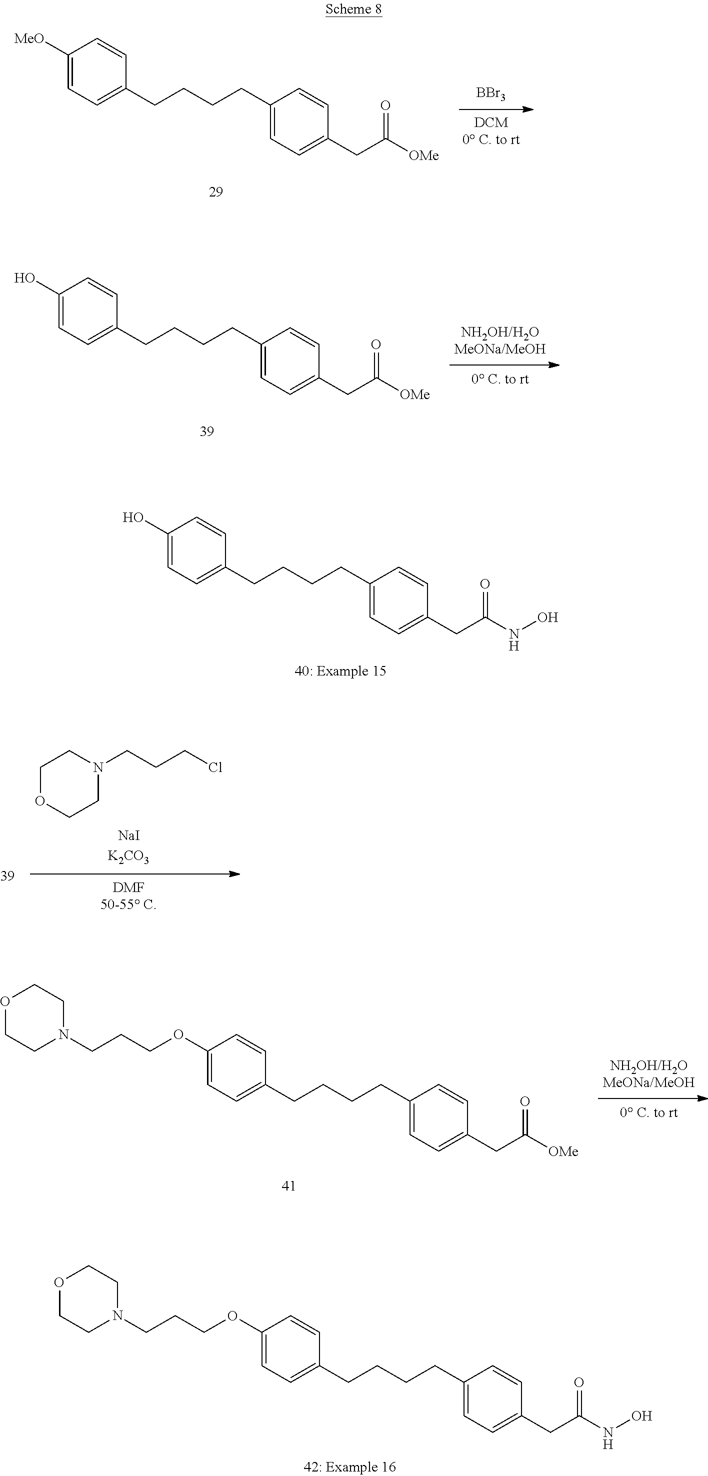

### Example 15

### N-Hydroxy-2-(4-(4-(4-hydroxyphenyl)butyl)phenyl)acetamide (40, Example 15)

### Step 1. Methyl 2-(4-(4-(4-hydroxyphenyl)butyl)phenyl)acetate (39)

To a stirred solution of compound 29 (687 mg, 2.2 mmol) in DCM (20 ml) at 0° C. under nitrogen was added slowly a solution of 1M boron tribromide in DCM (6.62 ml, 6.62 mmol) and the reaction mixture was stirred to rt over 3 hrs, cooled-down to 0° C., quenched by addition of methanol and water, and extracted with DCM. The organic extract was dried over anhydrous magnesium sulfate, filtered and concentrated. The residue was purified by Biotage (SiliaFlash 80 g cartridge; MeOH/DCM: 0/100 to 02/98 over 20 CV) to afford the title compound 39 (465 mg, 1.56 mmol, 70% yield) as a pale yellow sticky oil. MS (m/z): 321.4 (M+Na).

### Step 2. N-Hydroxy-2-(4-(4-(4-hydroxyphenyl)butyl)phenyl)acetamide (40, Example 15)

To a stirred solution of compound 39 (100 mg, 0.335 mmol) in MeOH (5 ml) under nitrogen at 0° C. were added a 50% aqueous solution of hydroxylamine (205 μl, 3.35 mmol) and a 25% wt/wt solution of sodium methoxide in MeOH (383 μl, 1.68 mmol). The reaction mixture was stirred from 0° C. to rt over 3 hrs, cooled-down to 0° C., diluted with water, and neutralyzed to pH 7-8 with 1 N HCl. The solid was collected by filtration, rinsed with water, dried and purified by Biotage (Snap 25 g cartridge; MeOH/DCM: 1/99 to 20/80 over 30 CV) to afford the title compound 40 (47 mg, 0.157 mmol, 46% yield) as an off-white fluffy solid. ¹H NMR (400 MHz, DMSO-d₆) δ (ppm): 10.60 (bs, 1H), 9.08 (s, 1H), 8.77 (bs, 1H), AB system (δ_{A}=7.13, δ_{B}=7.08, J_{AB}=8.0 Hz, 4H), A′B′ system (δ_{A′}=6.94, δ_{B′}=6.64, J_{A′B′}=8.4 Hz, 4H), 3.22 (s, 2H), 2.54 (t, J=7.1 Hz, 2H), 2.46 (t, J=7.0 Hz, 2H), 1.59-1.42 (m, 4H). MS (m/z): 300.2 (M+H).

### Example 16

### N-Hydroxy-2-(4-(4-(4-(3-morpholinopropoxy)phenyl)butyl)phenyl)acetamide (42, Example 16)

### Step 1. Methyl 2-(4-(4-(4-(3-morpholinopropoxy)phenyl)butyl)phenyl)acetate (41)

To a stirred solution of compound 39 (100 mg, 0.33 mmol) and 4-(3-chloropropyl)morpholine (82 mg, 0.50 mmol) in DMF (5 ml) under nitrogen at rt were added sodium iodide (10 mg, 0.07 mmol) and potassium carbonate (232 mg, 1.68 mmol). The reaction mixture was stirred at 50-55° C. overnight, cooled-down to rt, and partitioned between AcOEt and water. After separation the organic layer was successively washed with a saturated aqueous solution of sodium bicarbonate, a saturated aqueous solution of ammonium chloride, water and brine, dried over anhydrous MgSO₄, filtered and concentrated. The residue was purified by Biotage (Snap 25 g cartridge; MeOH/DCM: 0/100 to 5/95 over 20 CV) to afford the title compound 41 (125 mg, 0.29 mmol, 88% yield) as a colorless sticky oil. MS (m/z): 426.34 (M+H). The material was used in the next step with no further purification.

### Step 2. N-Hydroxy-2-(4-(4-(4-(3-morpholinopropoxy)phenyl)butyl)phenyl) acetamide (42, Example 16)

To a stirred solution of compound 41 (465 mg, crude, ca 66% purity) in MeOH (15 ml) under nitrogen at 0° C. were added a solution of hydroxylamine (884 μl, 14.42 mmol, 50% in water) and a solution of sodium methoxide (1.65 ml, 7.21 mmol, 25% in MeOH). The reaction mixture was allowed to warm from 0° C. to rt over 2 h and then stirred at rt for an additional 2.5 h, concentrated (not to dryness), diluted with water, cooled-down to 0° C. and neutralized to pH around 7-8 with 1 N HCl. The solid precipitate was collected by filtration, rinsed with water and air-dried. The dry material was purified by Biotage (reverse phase chromatography: Snap 30 g cartridge KP-C18-HS; MeOH/water: 20/80 to 95/5 over 50 CV, 40 ml/min). The desired fractions were combined, concentrated and dried to afford the desired product 42 (227 mg, 0.53 mmol, 73% yield) as a white sticky solid. ¹H NMR (400 MHz, DMSO-d₆) δ (ppm): mixture of rotamers, 10.61 (bs, 1H), 8.79 (bs, 1H), 7.21-6.97 (m, 6H), 6.80 (d, J=8.4 Hz, 2H), 3.94 (t, J=6.4 Hz, 2H), 3.60-3.52 (m, 4H), 3.21 (s, 2H), 2.59-2.50 (m, 4H are partially hidden by DMSO), 2.40 (t, J=7.2 Hz, 2H), 2.39-2.29 (m, 4H), 1.84 (quint, J=6.8 Hz, 2H), 1.59-1.46 (m, 4H). MS (m/z): 427.3 (M+H).

Compounds 43 and 44 (examples 17 and 18) were prepared in two steps by alkylatling compound 39 (Scheme 8) with the appropriate alkylating reagent similarly to compound 42 (Scheme 8).

TABLE 7
Characterization of compounds 43 and 44 (examples 17 and 18).

| Cpd | Ex. | Structure | Characterization |
|---|---|---|---|
| 43 | 17 | 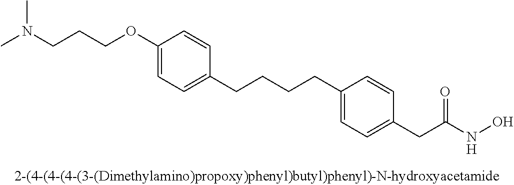 [SMILES: CN(C)CCCOc1ccc(CCCCc2ccc(CC(=O)NO)cc2)cc1] | ¹H NMR (400 MHz, MeOH-d₄) δ (ppm): mixture of rotamers, 1NH and 1OH are missing, 7.25 and 7.20 (2d, J = 8.2 Hz, 2H), 7.12- 6.98 (m, 4H), 6.85-6.78 (m, 2H), 4.07 (t, J = 5.9 Hz, 2H), 3.54-3.40 (m, 4H), 3.20 (s, 6H), 2.68-2.24 (m, 6H), 1.88-1.50 (m, 4H). MS (m/z): 386.2 (M + H). |
| 44 | 18 | 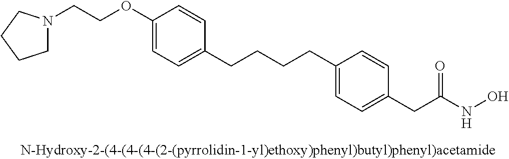 [SMILES: O=C(Cc1ccc(CCCCc2ccc(OCCN3CCCC3)cc2)cc1)NO] | ¹H NMR (400 MHz, MeOH-d₄) δ (ppm): mixture of rotamers, 1NH and 1OH are missing, AB system (δ_{A} = 7.20, δ_{B} = 7.04, J_{AB} = 8.1 Hz, 4H), A′B′ system (δ_{A′} = 7.09, δ_{B′} = 6.87, J_{A′B′} = 8.7 Hz, 4H), 4.55-4.50 (m, 2H), 3.75-3.69 (m, 2H), 3.59- 3.42 (m, 4H), 3.42 (s, 2H), 2.64-2.50 (m, 4H), 2.39- 2.25 (m, 2H), 2.12-2.00 (m, 2H), 1.66-1.51 (m, 4H). MS (m/z): 398.2 (M + H). |

### Example 19

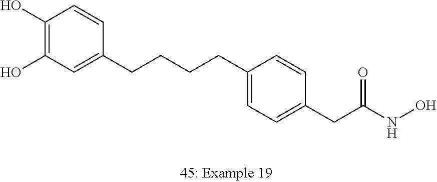 [SMILES: O=C(Cc1ccc(CCCCc2ccc(O)c(O)c2)cc1)NO]

### 2-(4-(4-(3,4-Dihydroxyphenyl)butyl)phenyl)-N-hydroxyacetamide 45 Example 19)

Compound 45 was prepared in four steps by following the procedures similar to the ones described above for the synthesis of compound 40 (Schemes 6 and 8). ¹H NMR (400 MHz, DMSO-d₆) δ (ppm):10.80-10.40 (m, 1H), 9.00-8.40 (m, 3H), AB system (δ_{A}=7.14, δ_{B}=7.08, J_{AB}=8.2 Hz, 4H), ABX system (δ_{A}=6.59, δ_{B}=6.39, δ_{X}=6.52, J_{AB}=8.0 Hz, J_{BX}=2.0 Hz, J_{AX}=0 Hz, 3H), 3.21 (s, 2H), 2.53 (t, J=7.1 Hz, 2H), 2.39 (t, J=7.1 Hz, 2H), 1.58-1.40 (m, 4H). MS (m/z): 316.1 (M+H) and 338.1 (M+Na).

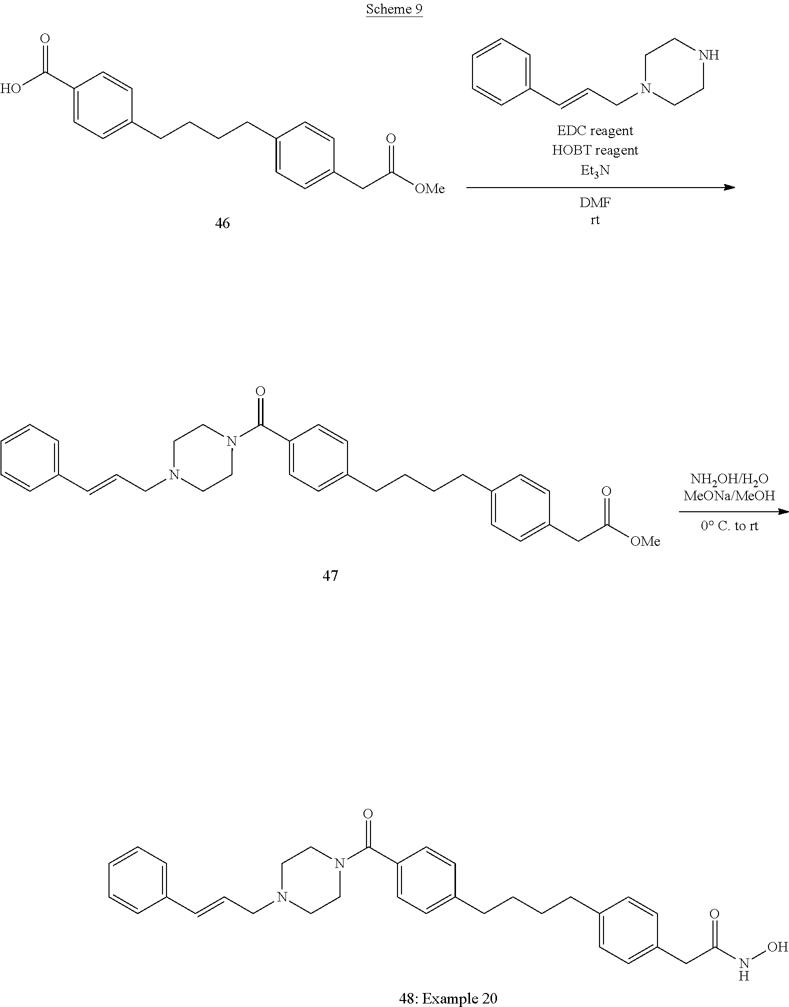 [SMILES: COC(=O)Cc1ccc(CCCCc2ccc(C(=O)O)cc2)cc1 ; C(=C\c1ccccc1)/CN1CCNCC1 ; COC(=O)Cc1ccc(CCCCc2ccc(C(=O)N3CCN(C/C=C/c4ccccc4)CC3)cc2)cc1 ; O=C(Cc1ccc(CCCCc2ccc(C(=O)N3CCN(C/C=C/c4ccccc4)CC3)cc2)cc1)NO]

### Example 20

### (E)-2-(4-(4-(4-(4-cinnamylpiperazine-1-carbonyl)phenyl)butyl)phenyl)-N-hydroxyacetamide (48, Example 20)

### Step 1. (E)-Methyl 2-(4-(4-(4-(4-cinnamylpiperazine-1-carbonyl)phenyl)butyl) phenyl)acetate (47)

To a stirred solution of compound 46 (115 mg, 0.35 mmol) [prepared in two steps by following the procedures similar to the ones described for the synthesis of compound 29 (Scheme 6) but using 4-iodobenzoic acid instead of 4-iodoanisole in the first step] in DMF (5 mL) under nitrogen were added trans-1-cinnamyl-piperazine (86 mg, 0.42 mmol), triethylamine (195 μl, 1.41 mmol), HOBt-monohydrate (59 mg, 0.39 mmol) and EDC-hydrochloride (203 mg, 1.06 mmol). The reaction mixture was stirred at rt overnight. The reaction mixture was then partitioned between AcOEt and a saturated aqueous solution of sodium bicarbonate. After separation, the organic layer was successively washed with a saturated aqueous solution of sodium bicarbonate, water, a saturated aqueous solution of ammonium chloride and brine, dried over anhydrous MgSO₄, filtered, and concentrated. The residue was purified by Biotage (Snap 25 g cartridge; MeOH/DCM: 0/100 to 5/95 over 30 CV), to afford the title compound 47 (145 mg, 0.28 mmol, 81% yield) as a pale yellow sticky solid. MS (m/z): 511.2 (M+H).

### Step 2. (E)-2-(4-(4-(4-(4-Cinnamylpiperazine-1-carbonyl)phenyl)butyl)phenyl)-N-hydroxyacetamide (48, Example 20)

To a stirred solution of compound 47 (145 mg, 0.28 mmol) in MeOH (20 ml) u at 0° C. were added a solution of 50% aqueous solution of hydroxylamine (348 μl, 5.68 mmol) and a solution of 25% wt/wt NaOMe solution in methanol (0.65 ml, 2.84 mmol). The reaction mixture was stirred at 0° C. for 1 hr, then rt for 1.5 hrs, concentrated, cooled-down to 0° C., diluted with water, neutralyzed to pH 7-8 with 1 N HCl. The solid was collected by filtration, rinsed with water and dried. The dry material was purified by Biotage (reverse phase: Snap 30 g cartridge KP-C18-HS: MeOH/water: 20/80 to 95/05 over 40 CV) to afford the title compound 48 (94 mg, 0.18 mmol, 64% yield) as a white fluffy solid. ¹H NMR (400 MHz, DMSO-d₆) δ (ppm): 10.61 (bs, 1H), 8.79 (bs, 1H), 7.46-7.40 (m, 2H), 7.35-7.19 (m, 7H), AB system (δ_{A}=7.14, δ_{B}=7.09, J_{AB}=8.2 Hz, 4H), 6.54 (d, J=15.8 Hz, 1H), 6.30 (dt, J=15.8, 6.6 Hz, 1H), 3.76-3.30 (m, 4H), 3.21 (s, 2H), 3.13 (d, J=6.7 Hz, 2H), 2.70-2.51 (m, 4H), 2.50-2.30 (m, 4H), 1.66-1.50 (m, 4H). MS (m/z): 512.3 (M+H).

Compounds 49-52 (examples 21-24) were prepared in two steps by following the procedures similar to the ones described above for the synthesis of compound 48 (Scheme 9) by using compound 46 as the key intermediate to couple with the appropriate amines.

TABLE 8
Characterization of compounds 49-52 (examples 21-24).

| Cpd | Ex. | Structure | Characterization |
|---|---|---|---|
| 49 | 21 | 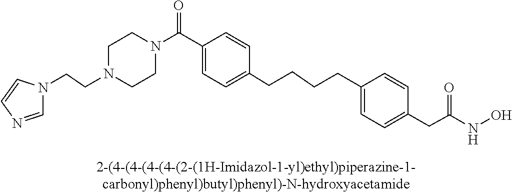 [SMILES: O=C(Cc1ccc(CCCCc2ccc(C(=O)N3CCN(CCn4ccnc4)CC3)cc2)cc1)NO] | ¹H NMR (400 MHz, DMSO-d₆) δ (ppm): 10.62 (bs, 1H), 8.80 (bs, 1H), 7.65-7.60 (m, 1H), AB system (δ_{A} = 7.28, δ_{B} = 7.23, J_{AB} = 8.2 Hz, 4H), 7.18 (t, J = 1.2 Hz, 1H), A′B′ system (δ_{A′} = 7.14, δ_{B′} = 7.09, J_{A′B′} = 8.2 Hz, 4H), 6.85 (t, J = 1.1 Hz, 1H), 4.07 (t, J = 6.4 Hz, 2H), 3.70-3.40 (m, 2H), 2H are hidden by water's peak, 3.22 (s, 2H), 2.70-2.51 (m, 6H), 2.50-2.30 (m, 4H), 1.65- 1.48 (m, 4H). MS (m/z): 490.3 (M + H). |
| 50 | 22 | 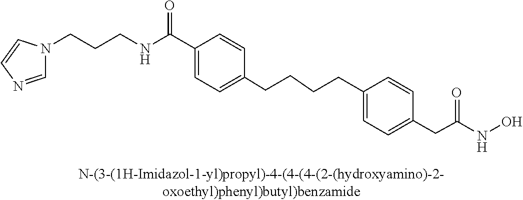 [SMILES: O=C(Cc1ccc(CCCCc2ccc(C(=O)NCCCn3ccnc3)cc2)cc1)NO] | ¹H NMR (400 MHz, DMSO-d₆) δ (ppm): 10.62 (bs, 1H), 8.81 (bs, 1H), 8.42 (t, J = 5.7 Hz, 1H), AB system (δ_{A} = 7.74, δ_{B} = 7.26, J_{AB} = 8.2 Hz, 4H), 7.67-7.63 (m, 1H), 7.20 (t, J = 1.2 Hz, 1H), A′B′ system (δ_{A′} = 7.14, δ_{B′} = 7.09, J_{A′B′} = 8.2 Hz, 4H), 6.89 (t, J = 1.1 Hz, 1H), 4.01 (t, J = 6.9 Hz, 2H), 3.26-3.16 (m, 4H), 2.64 (t, J = 7.0 Hz, 2H), 2.56 (t, J = 6.9 Hz, 2H), 1.94 (quint, J = 6.8 Hz, 2H), 1.64- 1.47 (m, 4H). MS (m/z): 435.2 (M + H). |
| 51 | 23 | 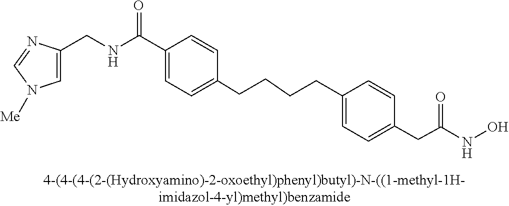 [SMILES: Cn1cnc(CNC(=O)c2ccc(CCCCc3ccc(CC(=O)NO)cc3)cc2)c1] | ¹H NMR (400 MHz, DMSO-d₆) δ (ppm): 10.61 (bs, 1H), 8.80 (bs, 1H), 8.70 (t, J = 5.7 Hz, 1H), AB system (δ_{A} = 7.77, δ_{B} = 7.24, J_{AB} = 8.3 Hz, 4H), 7.47 (bd, J = 1.0 Hz, 1H), A′B′ system (δ_{A′} = 7.14, δ_{B′} = 7.09, J_{A′B′} = 8.0 Hz, 4H), 6.92 (bd, J = 1.2 Hz, 1H), 4.30 (d, J = 5.5 Hz, 2H), 3.58 (s, 3H), 3.21 (s, 2H), 2.63 (t, J = 7.0 Hz, 2H), 2.56 (t, J = 6.8 Hz, 2H), 1.64-1.48 (m, 4H). MS (m/z): 421.1 (M + H). |
| 52 | 24 | 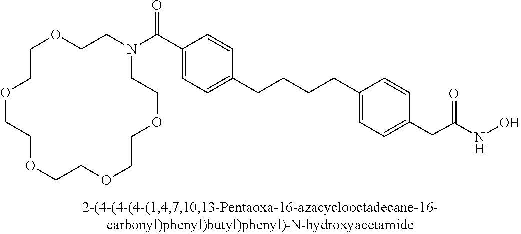 [SMILES: O=C(Cc1ccc(CCCCc2ccc(C(=O)N3CCOCCOCCOCCOCCOCC3)cc2)cc1)NO] | ¹H NMR (400 MHz, DMSO-d₆) δ (ppm): 10.60 (bs, 1H), 8.78 (bs, 1H), AB system (δ_{A} = 7.25, δ_{B} = 7.21, J_{AB} = 8.2 Hz, 4H), A′B′ system (δ_{A′} = 7.13, δ_{B′} = 7.08, J_{A′B′} = 8.2 Hz, 4H), 3.70-3.34 (m, 24H), 3.20 (s, 2H), 2.66-2.51 (m, 4H), 1.64-1.48 (m, 4H). MS (m/z): 573.4 (M + H). |

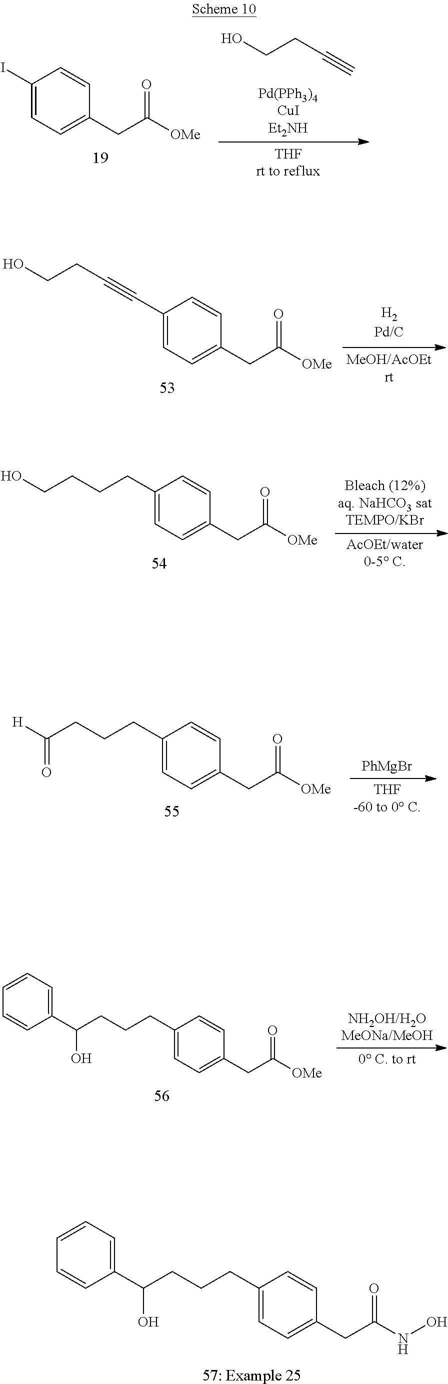

### Example 25

### N-Hydroxy-2-(4-(4-hydroxy-4-phenylbutyl)phenyl)acetamide (57, Example 25)

### Step 1. Methyl 2-(4-(4-hydroxybut-1-ynyl)phenyl)acetate (53)

To a stirred degassed solution of 3-butyn-1-ol (1.587 g, 22.64 mmol) and methyl 2-(4-iodophenyl)acetate (19) (5.00 g, 18.11 mmol) in THF (50 mL) at rt under nitrogen were added CuI (172 mg, 0.91 mmol), Pd(PPh₃)₄ (523 mg, 0.45 mmol) and diethylamine (5.64 ml, 54.3 mmol). The reaction mixture was stirred at rt for 3 h, heated to reflux overnight, then cooled to rt, diluted with AcOEt, successively washed with water, a saturated aqueous solution of ammonium chloride, water and brine, dried over anhydrous MgSO₄, filtered and concentrated. The residue was purified by Biotage (Snap 100 g cartridge; AcOEt/hexanes: 5/95 to 40/60 over 30 CV, 254 nm for wavelength collection), to afford the title compound 53 (3.23 g, 14.80 mmol, 82%) as an orange sticky oil.

### Step 2. Methyl 2-(4-(4-hydroxybutyl)phenyl)acetate (54)

To a stirred degassed solution of compound 53 (3.23 g, 14.80 mmol) in methanol/ethyl acetate (50/25 mL) was added wet 10% palladium on carbon Degussa type (3.15 g, 2.96 mmol) and the reaction mixture was stirred overnight under was atmosphere of hydrogen, filtered through celite, rinsed with ethyl acetate and concentrated to afford the title compound 54 (3.10 g, 13.95 mmol, 94% yield) as a colorless oily liquid. The crude product was used in the next step without any further purification. MS (m/z): 245.0 (M+Na).

### Step 3. Methyl 2-(4-(4-oxobutyl)phenyl)acetate (55)

To a stirred solution of compound 54 (3.10 g, 13.95 mmol) in AcOEt (30 ml) at 0° C. were added a solution of potassium bromide in water (166 mg, 1.4 mmol, in 0.37 mL), a solution of TEMPO in ethyl acetate (44 mg, 0.28 mmol, in 1 mL), and dropwise, a mixture of commercial bleach (8.61 ml) and a saturated aqueous solution of sodium bicarbonate (3.5 mL), respectively. The oxidation reaction was monitored by TLC. More saturated aqueous solution of sodium bicarbonate (10 mL) and commercial bleach (55 mL) were added in order to complete the conversion into the desired product. Then, the reaction mixture was quenched with an aqueous solution of 1.05 M sodium thiosulfate, and diluted with ethyl acetate. After separation, the organic layer was successively washed with an aqueous solution of 1.05 M sodium thiosulfate, water and brine, dried over anhydrous MgSO₄, filtered and concentrated. The residue was purified by Biotage (SiliaFlash 80 g cartridge; AcOEt/hexanes: 1/99 to 20/80 over 30 CV, 254 nm for wavelength collection), to afford the title compound 55 (1.41 g, 6.40 mmol, 46% yield) as a colorless oily liquid.

### Step 4. Methyl 2-(4-(4-hydroxy-4-phenylbutyl)phenyl)acetate (56)

To a stirred solution of compound 55 (1.41 g, 6.40 mmol) in anhydrous THF (30 mL) under nitrogen atmosphere at −60° C. was added a solution of 1M phenylmagnesium bromide in THF (8.32 mL, 8.32 mmol). The reaction mixture was allowed to warm-up to −20° C. over 1.5 h, cooled-down to −40° C., and 2 ml of phenylmagnesium bromide in THF (2 mmol) were added again. The reaction mixture was allowed to warm-up to 0° C. over 1.5 h, quenched with a saturated aqueous solution of ammonium chloride, and extracted with ethyl acetate. The organic layer was successively washed with water and brine, dried over anhydrous MgSO₄, filtered and concentrated. The residue was purified by Biotage (Snap 50 g cartridge; AcOEt/hexanes: 1/99 to 30/70 over 30 CV, 220 nm for wavelength collection), to afford the title compound 55 (1.519 g, 5.09 mmol, 80% yield) as a colorless oily liquid.

### Step 5. N-Hydroxy-2-(4-(4-hydroxy-4-phenylbutyl)phenyl)acetamide (57, Example 25)

To a stirred solution of compound 56 (110 mg, 0.37 mmol) in MeOH (15 ml) at 0° C. were added a 50% aqueous solution of hydroxylamine (678 μl, 11.06 mmol) and a wt/wt 25% solution of sodium methoxide in MeOH (1.69 ml, 7.37 mmol). The reaction mixture was stirred at 0° C. for 2.5 h, concentrated, cooled-down to 0° C., diluted with water, neutralyzed to pH around 7-8 with 1 N HCl. The solid was collected by filtration, rinsed with water and dried. The dry material was purified by Biotage (reverse phase: Snap 30 g cartridge KP-C18-HS: MeOH/water: 10/90 to 95/05 over 50 CV, 220 nm for the wavelength collection), to afford the title compound 57 (27 mg, 0.09 mmol, 24% yield) as a white sticky solid. ¹H NMR (400 MHz, DMSO-d₆) δ (ppm): 10.62 (s, 1H), 8.80 (s, 1H), 7.33-7.25 (m, 4H), 7.24-7.16 (m, 1H), AB system (δ_{A}=7.13, δ_{B}=7.06, J_{AB}=8.0 Hz, 4H), 5.14 (d, J=4.3 Hz, 1H), 4.56-4.47 (m, 1H), 3.21 (s, 2H), 2.58-2.50 (m, 2H), 1.68-1.40 (m, 4H). MS (m/z): 322.1 (M+Na).

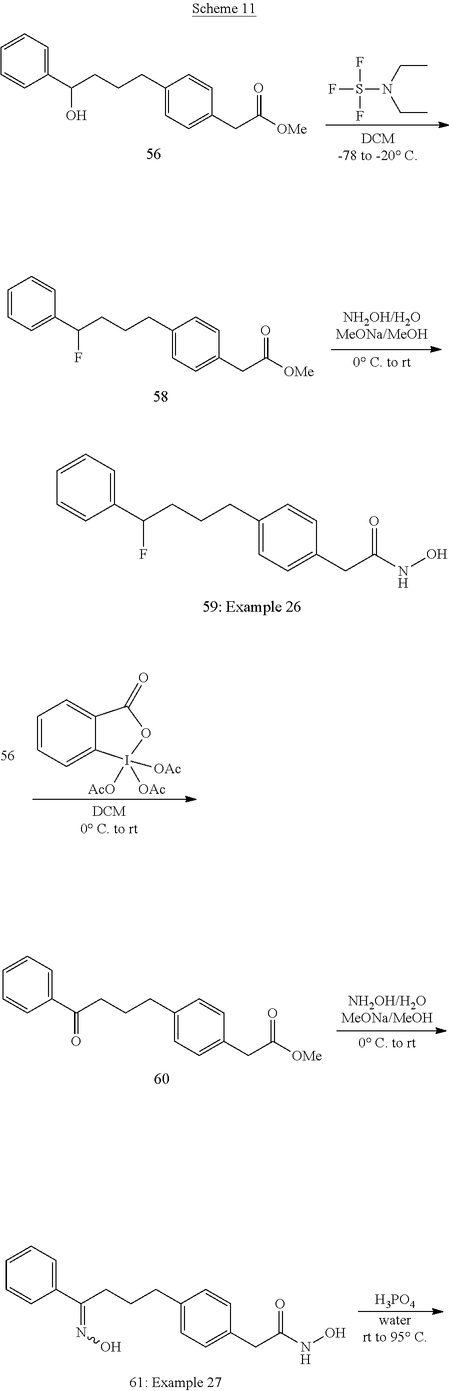

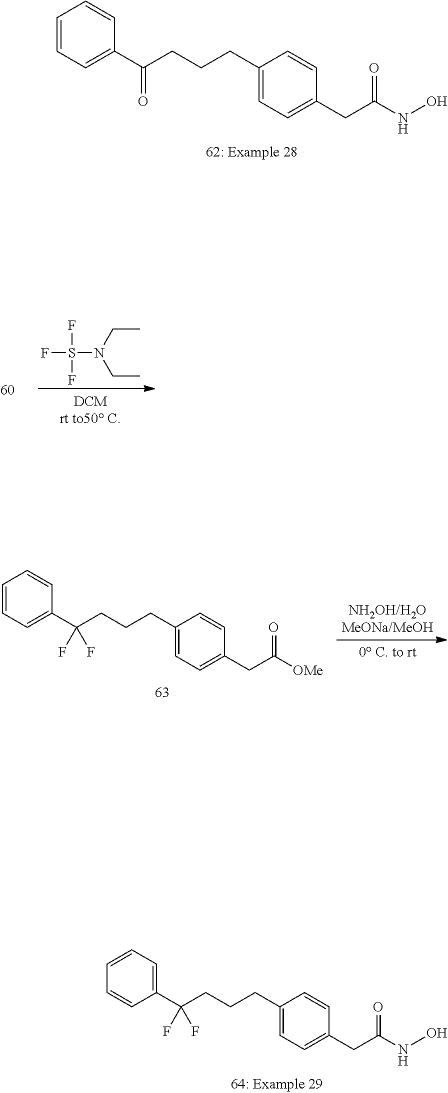

### Example 26

### 2-(4-(4-Fluoro-4-phenylbutyl)phenyl)-N-hydroxyacetamide (59, Example 26)

### Step 1. Methyl 2-(4-(4-fluoro-4-phenylbutyl)phenyl)acetate (58)

To a stirred solution in a plastic bottle of compound 56 (144 mg, 0.48 mmol) in DCM (10 ml) under nitrogen at −78° C. was added DAST (83 μl, 0.63 mmol). The reaction mixture was allowed to warm-up to −20° C. over 2 h, quenched by addition of saturated NH₄Cl, and diluted with DCM. After separation, the aqueous layer was extracted with DCM, and the combined organic layer was dried over anhydrous MgSO₄, filtered and concentrated. The residue was purified by Biotage (Snap 25 g cartridge, eluted with AcOEt/hexanes: 0/100 to 10/90 over 30 CV, 220 nm for the wavelength collection), to afford the title compound 58 (73 mg, 0.24 mmol, 50% yield) as a colorless sticky film/oil. MS (m/z): 323.15 (M+Na).

### Step 2. 2-(4-(4-Fluoro-4-phenylbutyl)phenyl)-N-hydroxyacetamide (59, Example 26)

Compound 59 was prepared in one step from compound 58 similarly to compound 57 (Scheme 10). ¹H NMR (400 MHz, DMSO-d₆) δ (ppm):10.62 (bs, 1H), 8.80 (bs, 1H), 7.45-7.27 (m, 5H), AB system (δ_{A}=7.15, δ_{B}=7.09, J_{AB}=8.1 Hz, 4H), 5.55 (ddd, J=47.7, 8.1, 4.8 Hz, 1H), 3.22 (s, 2H), 2.58 (t, J=7.5 Hz, 2H), 2.00-1.50 (m, 4H). MS (m/z): 282.1 (M+H−HF) and 324.1 (M+Na).

### Example 27

### N-Hydroxy-2-(4-(4-(hydroxyimino)-4-phenylbutyl)phenyl)acetamide (61, Example 27)

### Step 1. Methyl 2-(4-(4-oxo-4-phenylbutyl)phenyl)acetate (60)

To a stirred solution of compound 56 (1.21 g, 4.06 mmol) in DCM (40 ml) at 0° C. under nitrogen was added Dess-Martin periodinane (1.892 g, 4.46 mmol) in one portion and the reaction mixture was stirred at 0° C. for 2 h then at rt for 3 h. The reaction mixture was cooled-down to 0° C. and poured into 1N NaOH. After separation, the aqueous layer was extracted with DCM. The combined organic layer was dried over anhydrous MgSO₄, filtered, and concentrated. The residue was purified by Biotage (SiliaFlash 80 g cartridge; AcOEt/hexanes: 1/99 to 15/85 over 30 CV, 254 nm for wavelength collection), to afford the title compound 60 (952 mg, 3.21 mmol, 79% yield) as a colorless oily liquid. MS (m/z): 297.04 (M+NH).

### Step 2. N-Hydroxy-2-(4-(4-(hydroxyimino)-4-phenylbutyl)phenyl)acetamide (61, Example 27)

Compound 61 was prepared in one step from compound 60 similarly to compound 57 (Scheme 10). ¹H NMR (400 MHz, DMSO-d₆) δ (ppm): 10.62 (bs, 1H), 8.80 (bs, 1H), 7.45-7.27 (m, 5H), AB system (δ_{A}=7.15, δ_{B}=7.09, J_{AB}=8.1 Hz, 4H), 5.55 (ddd, J=47.7, 8.1, 4.8 Hz, 1H), 3.22 (s, 2H), 2.58 (t, J=7.5 Hz, 2H), 2.00-1.50 (m, 4H). MS (m/z): 313.13 (M+H).

### Example 28

### N-Hydroxy-2-(4-(4-oxo-4-phenylbutyl)phenyl)acetamide (62, Example 28)

To a stirred suspension of compound 61 (40 mg, 0.128 mmol) in water (10 ml) at rt was added a solution of 85% orthophosphoric acid (2 mL). The reaction mixture (a suspension) was heated at 95° C. for 30 min then cooled to rt. The solid was collected by filtration, rinsed with water and dried. The dry material was purified by reverse phase chromatography using Biotage (Snap 30 g cartridge KP-C18-HS: MeOH/water: 20/80 to 95/05 over 50 CV, 220 nm for the wavelength collection), to afford the title compound 62 (13.4 mg, 0.045 mmol, 35% yield) as an off-white fluffy solid. ¹H NMR (400 MHz, DMSO-d₆) δ (ppm): 10.62 (bs, 1H), 8.80 (bs, 1H), 7.94 (d, J=7.2 Hz, 2H), 7.63 (t, J=7.3 Hz, 1H), 7.52 (t, J=7.7 Hz, 2H), AB system (δ_{A}=7.17, δ_{B}=7.14, J_{AB}=8.1 Hz, 4H), 3.23 (s, 2H), 3.03 (t, J=7.1 Hz, 2H), 2.61 (t, J=7.6 Hz, 2H), 1.89 (quint, J=7.4 Hz, 2H). MS (m/z): 298.0 (M+H) and 320.0 (M+Na).

### Example 29

### 2-(4-(4,4-Difluoro-4-phenylbutyl)phenyl)-N-hydroxyacetamide (64, Example 29)

### Step 1. Methyl 2-(4-(4,4-difluoro-4-phenylbutyl)phenyl)acetate (63)

To a stirred solution of compound 60 (280 mg, 0.945 mmol) in DCM (1 mL) in a plastic bottle under nitrogen at rt was added DAST (1.04 ml, 7.56 mmol). The reaction mixture was heated at 45-50° C. overnight then cooled to rt. More DAST (1.04 ml, 7.56 mmol) was added, and the reaction mixture was heated at 50° C. for 3 days, then cooled-down to 0° C., poured dropwise into a mixture of water/ice (gas evolution) and extracted with DCM. The combined organic layer was dried over anhydrous MgSO₄, filtered, and concentrated. The residue was purified by Biotage (Snap 25 g cartridge: AcOEt/hexanes: 0/100 to 05/95 over 30 CV, 220 nm for the wavelength collection), to afford the title compound 64 (156 mg, 0.49 mmol, 51% yield) as a colorless oily liquid. MS (m/z): 319.0 (M+H).

### Step 2. 2-(4-(4,4-Difluoro-4-phenylbutyl)phenyl)-N-hydroxyacetamide (64, Example 29)

Compound 64 was prepared in one step from compound 63 similarly to compound 57 (Scheme 10). ¹H NMR (400 MHz, DMSO-d₆) δ (ppm): 10.62 (bs, 1H), 8.80 (bs, 1H), 7.48 (bs, 5H), AB system (δ_{A}=7.15, δ_{B}=7.07, J_{AB}=8.2 Hz, 4H), 3.22 (s, 2H), 2.56 (t, J=7.6 Hz, 2H), 2.27-2.09 (m, 2H), 1.66-1.53 (m, 2H). MS (m/z): 313.13 (M+H). MS (m/z): 280.03 (M+H-2HF) and 342.04 (M+Na).

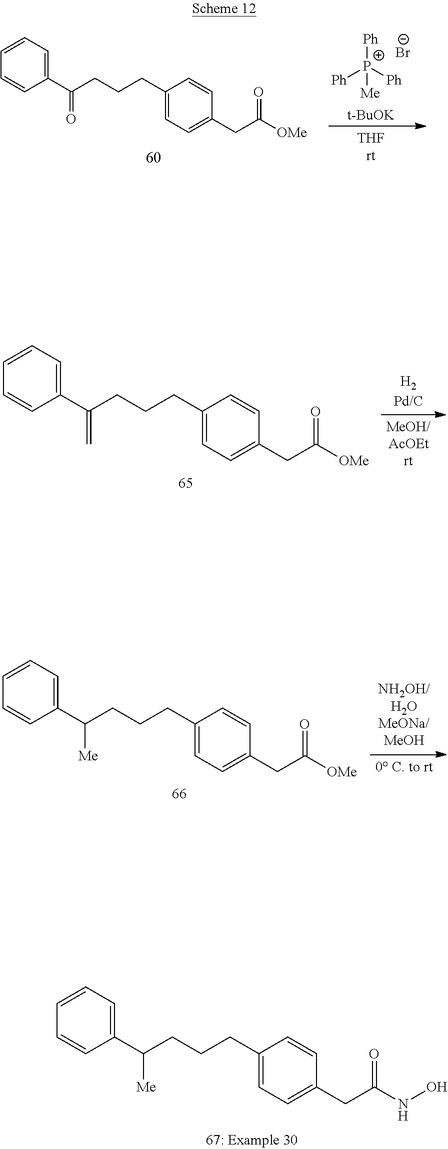

### Example 30

### N-Hydroxy-2-(4-(4-phenylpentyl)phenyl)acetamide (67, Example 30)

### Step 1. Methyl 2-(4-(4-phenylpent-4-enyl)phenyl)acetate (65)

To a stirred suspension of methyltriphenylphosphonium bromide (443 mg, 1.215 mmol) in annhydrous THF (10 ml) at rt under nitrogen was added potassium tert-butoxide (155 mg, 1.32 mmol) in one portion and the reaction mixture was stirred at rt for 30 min, before compound 60 (300 mg, 1.01 mmol) in anhydrous THF (10 ml) was added. The reaction mixture was stirred at rt overnight; more methyltriphenylphosphonium bromide (200 mg) and potassium tert-butoxide (120 mg) were added. The reaction mixture was stirred at rt for 24 hrs, cooled-down to 0° C., quenched with a saturated aqueous solution of ammonium chloride and extracted with AcOEt. The organic layer was successively washed with saturated NH₄Cl, saturated NaHCO₃, water and brine, dried over anhydrous MgSO₄, filtered, and concentrated. The residue was purified twice by Biotage (Snap 25 g cartridge; AcOEt/hexanes: 0/100 to 5/95 over 30 CV, 254 nm for wavelength collection), to afford the title compound 65 (70 mg, 0.238 mmol, 23% yield) as a colorless oily liquid. MS (m/z): 317.15 (M+Na).

### Step 2. Methyl 2-(4-(4-phenylpentyl)phenyl)acetate (66)

To a degassed stirred solution of compound 65 (70 mg, 0.238 mmol) in a mixture of methanol/AcOEt (5 ml/5 ml) was added wet 10% palladium on carbon Degussa type (51 mg, 0.048 mmol) and the reaction mixture was stirred in an atmosphere of hydrogen overnight. The reaction mixture was then filtered through a celite pad, rinsed with ethyl acetate and concentrated to afford the title compound 66 as a colorless oily liquid. The crude product was used in the next step without any further purification. MS (m/z): 319.08 (M+Na).

### Step 3. N-Hydroxy-2-(4-(4-phenylpentyl)phenyl)acetamide (67, Example 30)

Compound 67 was prepared in one step from compound 66 similarly to compound 57 (Scheme 10). ¹H NMR (400 MHz, DMSO-d₆) δ (ppm): 10.61 (bs, 1H), 8.80 (bs, 1H), 7.31-7.22 (m, 2H), 7.21-7.12 (m, 3H), AB system (δ_{A}=7.12, δ_{B}=7.04, J_{AB}=8.1 Hz, 4H), 3.20 (s, 2H), 2.69 (hex, J=7.0 Hz, 1H), 2H are hidden by DMSO, 1.52 (quint, J=7.0 Hz, 2H), 1.49-1.28 (m, 2H), 1.17 (d, J=7.0 Hz, 3H). MS (m/z): 298.2 (M+H) and 320.2 (M+Na).

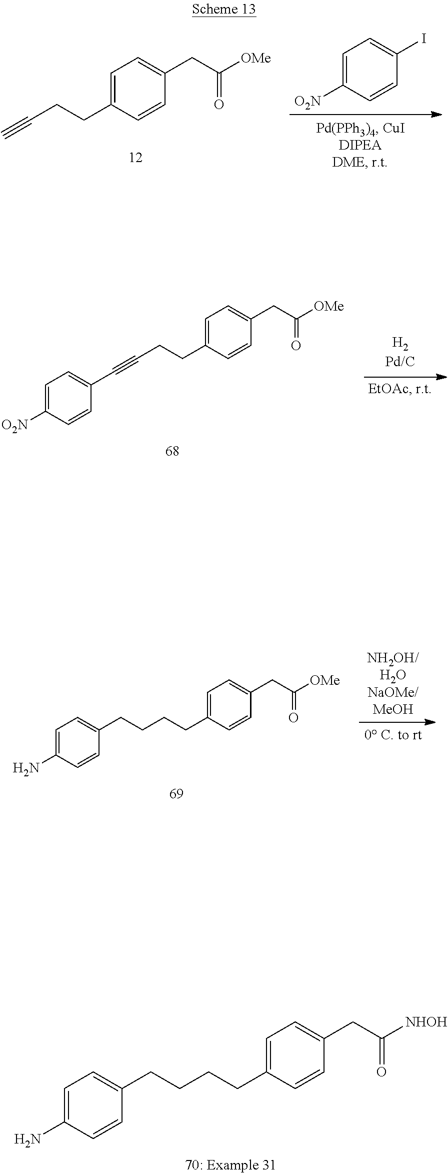

### Example 31

### 2-(4-(4-(4-Aminophenyl)butyl)phenyl)-N-hydroxyacetamide (70, Example 31)

### Step 1: Methyl 2-(4-(4-(4-nitrophenyl)but-3-ynyl)phenyl)acetate (68)

To a degassed solution of 1-iodo-4-nitrobenzene (718 mg, 2.88 mmol), Pd(PPh₃)₄ (167 mg, 0.14 mmol), CuI (82 mg, 0.43 mmol) and DIPEA (1.51 mL, 8.65 mmol) in DME (40 mL) was added methyl 2-(4-(but-3-ynyl)phenyl)acetate (12) (0.70 g, 3.46 mmol, WO 2008/074132 A1). The reaction mixture was stirred at room temperature for 2 hrs then concentrated. The residue was partitioned between HCl 1N and EtOAc. The organic phase was collected, washed with brine, dried over sodium sulfate, filtered and concentrated. The residue was purified by Biotage (SNAP 25 g cartridge; 0 to 30% of EtOAc in hexanes over 20 CV) to afford the title compound 68 (873 mg, 2.70 mmol, 94% yield) as a yellow solid. ¹H NMR (400 MHz, DMSO-d₆) δ (ppm): 8.22-8.16 (m, 2H), 7.62-7.56 (m, 2H), 7.27 (d, J=7.2 Hz, 2H), 7.20 (d, J=7.2 Hz, 2H), 3.64 (s, 2H), 3.60 (s, 3H), 2.86 (d, J=6.8 Hz, 2H), 2.77 (d, J=6.8 Hz, 2H).

### Step 2: Methyl 2-(4-(4-(4-aminophenyl)butyl)phenyl)acetate (69)

A solution of the nitro compound 68 (873 mg, 2.70 mmol) in EtOAc (50 mL) was hydrogenated (1 atm pressure) over Pd/C Degussa type 101 (287 mg, 0.27 mmol) for 21 hrs. The reaction mixture was then filtered through a Celite pad, washed with MeOH and concentrated to afford the title compound 69 (747 mg, 2.51 mmol, 93% yield) as a brown oil. ¹H NMR (400 MHz, DMSO-d₆) δ (ppm): 7.14 (d, J=8.0 Hz, 2H), 7.10 (d, J=8.0 Hz, 2H), 6.80 (d, J=8.0 Hz, 2H), 6.46 (d, J=8.0 Hz, 2H), 4.82 (bs, 2H), 3.61 (s, 2H), 3.59 (s, 3H), 2.54 (t, J=7.2 Hz, 2H), 2.40 (t, J=7.2 Hz, 2H), 1.56-1.45 (m, 4H).

### Step 3: 2-(4-(4-(4-Aminophenyl)butyl)phenyl)-N-hydroxyacetamide (70, Example 31)

To a solution of the amine 69 (113 mg, 0.38 mmol) in MeOH (5 mL) was added a 50% aqueous hydroxylamine solution (0.233 mL, 3.80 mmol) and a 25% w/w solution of sodium methoxide in MeOH (0.43 mL, 1.90 mmol) at 0° C. The reaction mixture was stirred for 1 hr at 0° C., at room temperature for 1 h then concentrated. To the residue were added water and HCl 1N. The acidified solution was extracted with DCM. The organic phase was washed with brine, dried over sodium sulfate, filtered and concentrated. The residue was purified by Biotage (SNAP 10 g cartridge; 0 to 10% of MeOH in DCM over 20 CV), to afford the title compound 70 (38.4 mg, 0.13 mmol, 34%) as a white solid. ¹H NMR (400 MHz, DMSO-d₆) δ (ppm): 10.6 (s, 1H), 8.79 (s, 1H), 7.13 (d, J=8.0 Hz, 2H), 7.08 (d, J=8.0 Hz, 2H), 6.80 (d, J=8.0 Hz, 2H), 6.45 (d, J=8.0 Hz, 2H), 4.79 (bs, 2H), 3.21 (s, 2H), 2.60-2.51 (m, 2H), 2.40 (t, J=8.0 Hz, 2H), 1-58-1.42 (m, 4H). MS (m/z): 299.1 (M+H).

Compounds 71-72 (examples-32-33) were prepared in three steps by following the procedures similar to the ones described above for the synthesis of compound 70 (Scheme 13) by using compound 12 as the starting material and replacing the 1-iodo-4-nitrobenzene with the 1-iodo-3-nitro- or 1-iodo-2-nitrobenzenes, respectively.

TABLE 9
Characterization of compounds 71-72 (examples 32-33).

| Cpd | Ex. | Structure | Characterization |
|---|---|---|---|
| 71 | 32 | 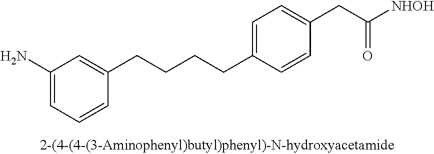 | ¹H NMR (400 MHz, DMSO-d₆) δ (ppm): 10.62 (s, 1H), 8.80 (s, 1H), 7.14 (d, J = 8.0 Hz, 2H), 7.08 (d, J = 8.0 Hz, 2H), 6.87 (td, J = 1.2 and 8.0 Hz, 1H), 6.37- 6.28 (m, 3H), 4.90 (bs, 2H), 3.21 (s, 2H), 2.54 (t, J = 7.2 Hz, 2H), 2.41 (t, J = 7.2 Hz, 2H), 2.10-1.97 (m, 4H). MS (m/z): 299.0 (M + 1). |
| 72 | 33 | 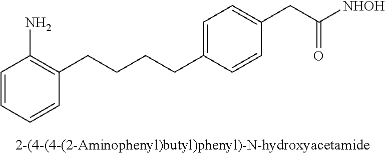 | ¹H NMR (400 MHz, DMSO-d₆) δ (ppm): 10.62 (s, 1H), 8.80 (s, 1H), 7.16-7.12 (m, 2H), 7.11-7.08 (m, 2H), 6.89-6.83 (m, 2H), 6.60-6.56 (m, 1H), 6.48-6.44 (m, 1H), 4.76 (bs, 2H), 3.21 (s, 2H), 2.60-2.51 (m, 2H), 2.44-2.38 (m, 2H), 1.62-1.55 (m, 2H), 1.55-1.47 (m, 2H). MS (m/z): 299.1 (M + 1). |

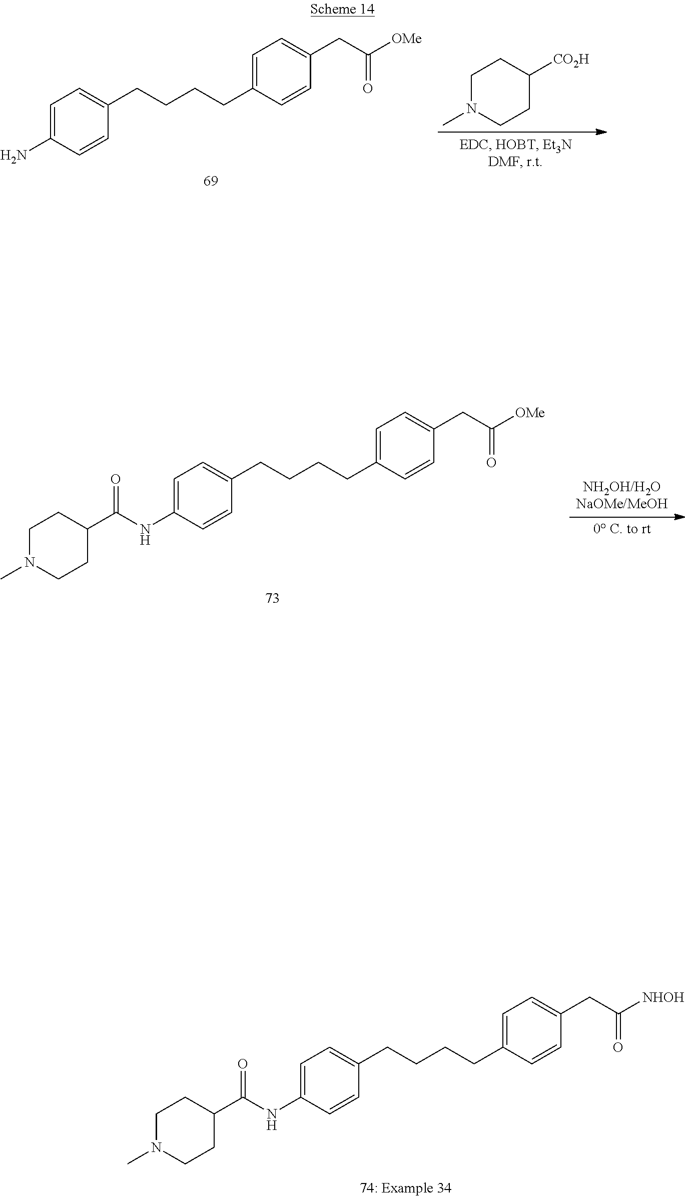

### Example 34

### N-(4-(4-(4-(2-(Hydroxyamino)-2-oxoethyl)phenyl)butyl)phenyl)-1-methylpiperidine-4-carboxamide (74, Example 34)

### Step 1. Methyl 2-(4-(4-(4-(1-methylpiperidine-4-carboxamido)phenyl)butyl)phenyl)acetate (73)

EDC xHCl (195 mg, 1.01 mmol) was added to a solution of the amine 69 (110 mg, 0.37 mmol), 1-methylpiperidine-4-carboxylic acid x HCl (80 mg, 0.44 mmol), Et₃N (0.20 mL, 1.48 mmol) and HOBt xH₂O (56.6 mg, 0.37 mmol) in DMF (10 mL). The reaction mixture was stirred at room temperature for 22 hrs, quenched by addition of water and saturated solution of ammonium chloride and extracted with EtOAc. The organic layer was washed with water, brine, dried over anhydrous sodium sulfate, filtered and concentrated. The residue was purified by Biotage (SNAP 10 g cartridge; MeOH/DCM: 0/100 to 15/85 over 20 CV), to afford the title compound 73 (106 mg, 0.25 mmol, 68%) as a yellow solid. ¹H NMR (400 MHz, DMSO-d₆) δ (ppm): 9.75 (s, 1H), 7.47 (d, J=8.0 Hz, 2H), 7.14 (d, J=8.0 Hz, 2H), 7.10 (d, J=8.0 Hz, 2H), 7.07 (d, J=8.0 Hz, 2H), 3.61 (s, 1H), 3.58 (s, 3H), 2.87-2.80 (m, 2H), 2.58-2.51 (m, 4H), 2.30-2.20 (m, 1H), 2.18 (s, 3H), 1-96-1.86 (m, 2H), 1.76-1.58 (m, 4H), 1.56-1.49 (m, 4H).

### Step 2. N-(4-(4-(4-(2-(Hydroxyamino)-2-oxoethyl)phenyl)butyl)phenyl)-1-methylpiperidine-4-carboxamide (74, Example 34)

A 25% w/w solution of sodium methoxide (0.29 ml, 1.25 mmol) was added to a solution of 73 (106 mg, 0.25 mmol) and 50% aqueous hydroxylamine solution (0.154 mL, 2.51 mmol) in MeOH (5 mL) at 0° C. The reaction was stirred for 1 hr at 0° C., for another 1 hr at room temperature then concentrated. The residue was partitioned between HCl 1N and DCM. The organic phase was collected, washed with brine, dried over anhydrous sodium sulfate, filtered and concentrated. The remaining material was purified by Biotage [SNAP 10 g cartridge; eluent 0 to 30% of MeOH (MeOH contained 2% of aqueous ammonia) in DCM over 20 CV]. The isolated solid was triturated with DCM to afford the title compound 74 (15.9 mg, 0.03 mmol, 14%) as a white solid. ¹H NMR (400 MHz, DMSO-d₆) δ (ppm): 10.64 (s, 1H), 10.03 (s, 1H), 9.93 (bs, 1H), 8.79 (s, 1H), 7.48 (d, J=8.4 Hz, 2H), 7.18-7.03 (m, 6H), 3.48-2.86 (m, 3H), 3.21 (s, 2H), 2.73 (s, 3H), 2.60-2.48 (m, 4H), 2.04-1.78 (m, 4H), 1.58-1.48 (m, 4H). MS (m/z): 424.2 (M+H).

Compounds 75-86 (examples 35-46) were prepared starting from the compound 69 or its two amino-isomers by following the procedures similar to the ones described above for the synthesis of compound 74 (Scheme 14), and replacing 1-methylpiperidine-4-carboxylic acid in the first step with the corresponding carboxylic acids.

TABLE 10
Characterization of compounds 75-86 (examples 35-46).

| Cpd | Ex. | Structure | Characterization |
|---|---|---|---|
| 75 | 35 | 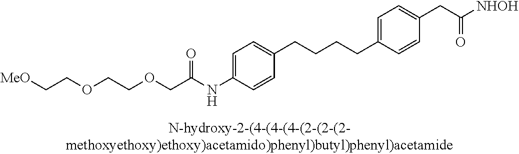 | ¹H NMR (400 MHz, DMSO-d₆) δ (ppm): 10.63 (bs, 1H), 9.50 (s, 1H), 8.81 (bs, 1H), 7.51 (d, J = 8.0 Hz, 2H), 7.18-7.03 (m, 6H), 4.04 (s, 2H), 3.68- 3.62 (m, 2H), 3.62-3.57 (m, 2H), 3.57- 3.53 (m, 2H), 3.47-3.43 (m, 2H), 3.22 (s, 3H), 3.21 (s, 2H), 2.58-2.49 (m, 4H), 1.58-1.48 (m, 4H). MS (m/z): 459.2 (M + 1). |
| 76 | 36 | 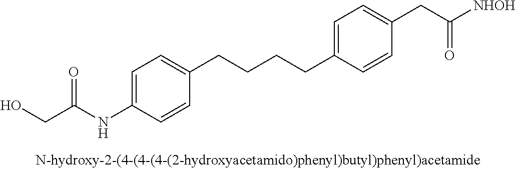 | ¹H NMR (400 MHz, DMSO-d₆) δ (ppm): 10.61 (s, 1H), 9.54 (s, 1H), 8.78 (s, 1H), 7.56 (d, J = 8.4 Hz, 2H), 7.18- 7.05 (m, 6H), 5.63 (t, J = 6.0 Hz, 1H), 3.96 (d, J = 6.0 Hz, 2H), 3.21 (s, 2H), 2.61-2.49 (m, 4H), 1.60-1.49 (m, 4H). MS (m/z): 357.1 (M + 1). |
| 77 | 37 | 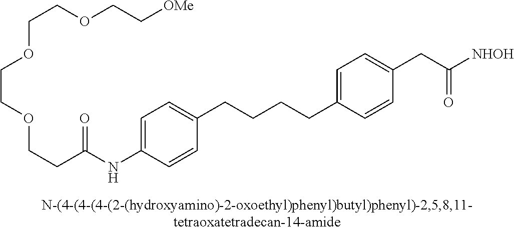 | ¹H NMR (400 MHz, DMSO-d₆) δ (ppm): 10.61 (s, 1H), 9.83 (s, 1H), 8.79 (s, 1H), 7.47 (d, J = 8.4 Hz, 2H), 7.17- 7.05 (m, 6H), 3.67 (t, J = 6.4 Hz, 2H), 3.53-3.44 (m, 10H), 3.42-3.37 (m, 2H), 3.21 (s, 5H), 2.57-2.50 (m, 6H), 1.56- 1.50 (m, 4H). MS (m/z): 517.2 (M + 1). |
| 78 | 38 | 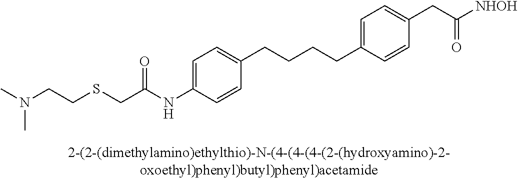 | ¹H NMR (400 MHz, DMSO-d₆) δ (ppm): 10.61 (s, 1H), 9.99 (s, 1H), 8.79 (s, 1H), 7.45 (d, J = 8.4 Hz, 2H), 7.19- 7.07 (m, 6H), 3.27 (s, 2H), 3.21 (s, 2H), 2.77-2.69 (m, 2H), 2.58-2.52 (m, 4H), 2.47-2.41 (m, 2H), 2.12 (s, 6H), 1.58- 1.50 (m, 4H). MS (m/z): 444.1 (M + 1). |
| 79 | 39 | 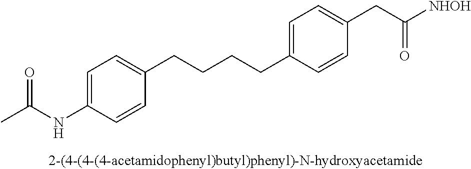 | ¹H NMR (400 MHz, DMSO-d₆) δ (ppm): 10.61 (s, 1H), 9.82 (s, 1H), 8.78 (s, 1H), 7.47-7.42 (m, 2H), 7.16-7.05 (m, 6H), 3.21 (s, 2H), 2.58-2.50 (m, 4H), 2.00 (s, 3H), 1.58-1.48 (m, 4H). MS (m/z): 341.2 (M + 1). |
| 80 | 40 | 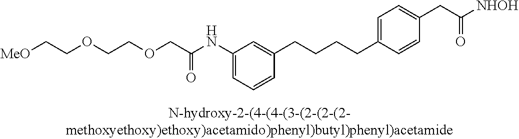 | ¹H NMR (400 MHz, DMSO-d₆) δ (ppm): 10.61 (bs, 1H), 9.50 (s, 1H), 8.79 (bs, 1H), 7.48-7.42 (m, 2H), 7.22- 7.07 (m, 5H), 6.91-6.87 (m, 1H), 4.05 (s, 2H), 3.68-3.64 (m, 2H), 3.62-3.57 (m, 2H), 3.57-3.54 (m, 2H), 3.47-3.43 (m, 2H), 3.22 (s, 3H), 3.21 (s, 2H), 2.60-2.52 (m, 4H), 1.60-1.52 (m, 4H). MS (m/z): 459.2 (M + 1). |
| 81 | 41 | 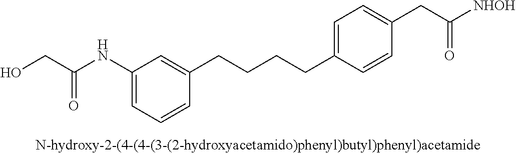 | ¹H NMR (400 MHz, DMSO-d₆) δ (ppm): 10.61 (s, 1H), 9.53 (s, 1H), 8.79 (s, 1H), 7.53-7.48 (m, 2H), 7.20-7.08 (m, 5H), 6.90-6.85 (m, 1H), 5.62 (t, J = 6.0 Hz, 1H), 3.96 (d, J = 6.0 Hz, 2H), 3.21 (s, 2H), 2.60-2.52 (m, 4H), 1.62- 1.50 (m, 4H). MS (m/z): 357.1 (M + 1). |
| 82 | 42 | 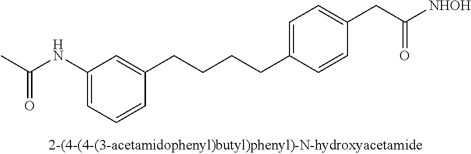 | ¹H NMR (400 MHz, DMSO-d₆) δ (ppm): 10.61 (s, 1H), 9.83 (s, 1H), 8.79 (s, 1H), 7.42-7.35 (m, 2H), 7.20-7.06 (m, 5H), 6.83 (d, J = 8.0 Hz, 1H), 3.21 (s, 2H), 2.60-2.51 (m, 4H), 2.01 (s, 3H), 1.61-1.48 (m, 4H). MS (m/z): 341.2 (M + 1). |
| 83 | 43 | 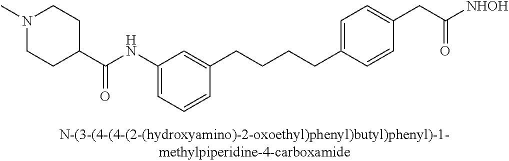 | ¹H NMR (400 MHz, DMSO-d₆) δ (ppm): 10.66 (s, 1H), 10.04 (s, 1H), 10.03 (bs, 1H), 8.80 (s, 1H), 7.45-7.38 (m, 2H), 7.20-7.08 (m, 5H), 6.85 (d, J = 7.6 Hz, 1H), 3.48-3.34 (m, 2H), 3.22 (s, 2H), 3.10-2.84 (m, 2H), 2.72 (s, 3H), 2.64-2.52 (m, 5H), 2.22-1.80 (m, 4H), 160-1.50 (m, 4H). MS (m/z): 424.1 (M + 1). |
| 84 | 44 | 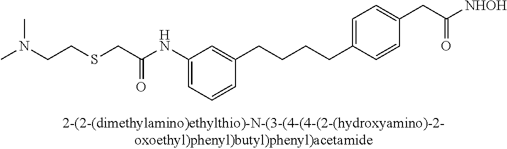 | ¹H NMR (400 MHz, DMSO-d₆) δ (ppm): 10.61 (s, 1H), 10.00 (s, 1H), 8.80 (s, 1H), 7.40-7.36 (m, 2H), 7.21- 7.07 (m, 5H), 6.86 (d, J = 7.6 Hz, 1H), 3.27 (s, 2H), 3.21 (s, 2H), 2.75-2.59 (m, 2H), 2.59-2.52 (m, 4H), 2.46-2.41 (m, 2H), 2.11 (s, 6H), 1.59-1.52 (m, 4H). MS (m/z): 444.1 (M + 1). |
| 85 | 45 | 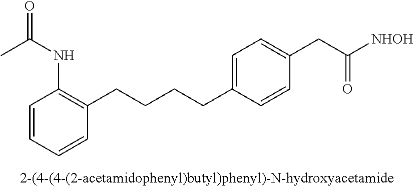 | ¹H NMR (400 MHz, DMSO-d₆) δ (ppm): 10.61 (s, 1H), 9.27 (s, 1H), 8.79 (s, 1H), 7.31 (d, J = 7.6 Hz, 1H), 7.22- 7.06 (m, 7H), 3.21 (s, 2H), 2.62-2.51 (m, 4H), 2.01 (s, 3H), 1.61-1.46 (m, 4H). MS (m/z): 341.1 (M + 1). |
| 86 | 46 | 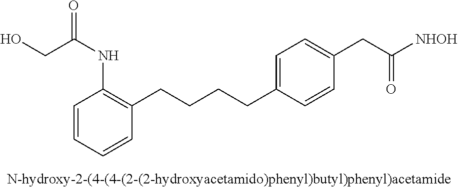 | ¹H NMR (400 MHz, DMSO-d₆) δ (ppm): 9.07 (s, 1H), 7.63 (d, J = 8.0 Hz, 1H), 7.23-7.06 (m, 7H), 3.99 (s, 2H), 3.21 (s, 2H), 2.64-2.52 (m, 4H), 1.62- 1.48 (m, 4H). MS (m/z): 357.0 (M + 1). |

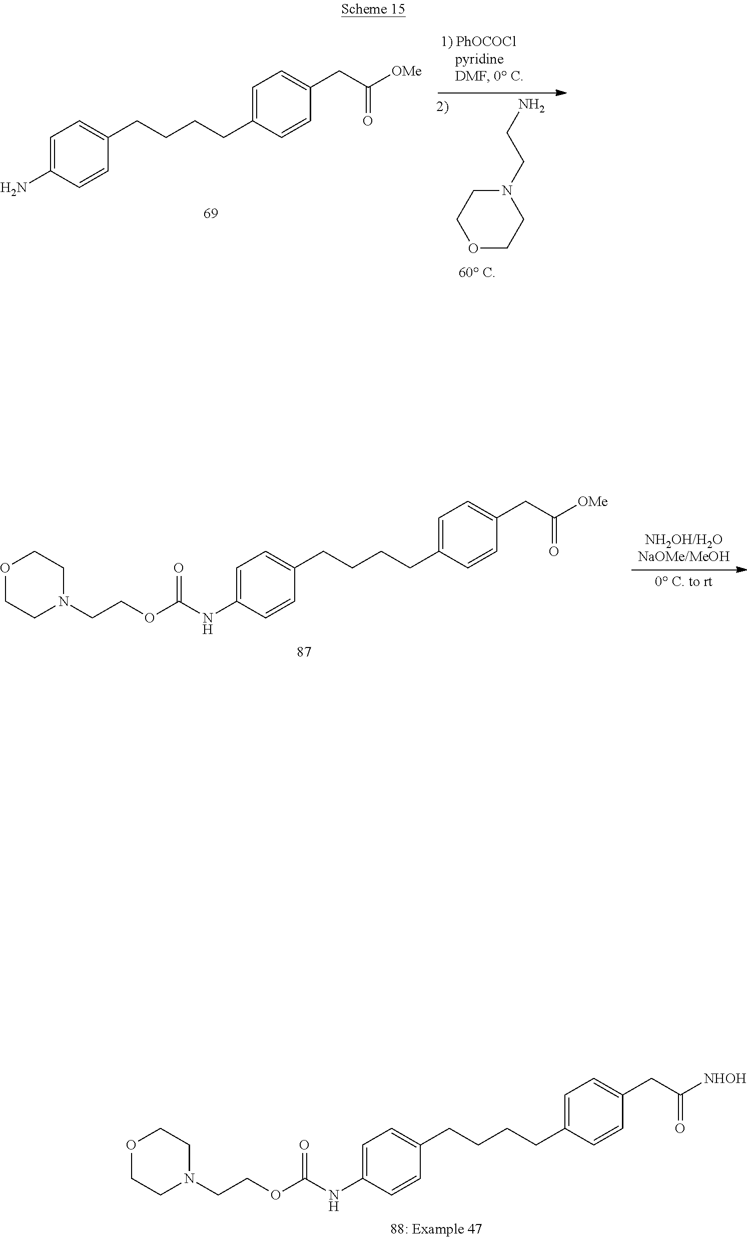

### Example 47

### 2-Morpholinoethyl 4-(4-(4-(2-(hydroxyamino)-2-oxoethyl)phenyl)butyl) phenylcarbamate (88, Example 47)

### Step 1.2-Morpholinoethyl 4-(4-(4-(2-(hydroxyamino)-2-oxoethyl)phenyl)butyl)phenylcarbamate (87)

To a solution of the amine 69 (100 mg, 0.34 mmol) and pyridine (54 μl, 0.67 mmol) in DMF (10 mL) at 0° C. was added phenyl chloroformate (51 μl, 0.40 mmol). The reaction mixture was stirred for 1 h at 0° C. before 4-(2-aminoethyl)-morpholine (110 μl, 0.84 mmol) was added. The combined reaction mixture was heated at 60° C. for 4 hrs. More 4-(2-aminoethyl)-morpholine (110 μl, 0.84 mmol) was added and the reaction mixture was heated at 60° C. for 2 days. After cooling to room temperature, the reaction was quenched by addition of water and saturated solution of ammonium chloride and extracted with EtOAc. The organic layer was washed with water, brine, dried over sodium sulfate, filtered and concentrated. The residue was purified by Biotage (SNAP 10 g cartridge; MeOH/DCM: 0/100 to 10/90 over 20 CV), to afford the title compound 87 (100 mg, 0.22 mmol, 66%) as a white solid. MS (m/z): 454.3 (M+H)

### Step 2.2-Morpholinoethyl 4-(4-(4-(2-(hydroxyamino)-2-oxoethyl)phenyl)butyl)phenylcarbamate (88, Example 47)

Sodium methoxide 25% w/w solution in MeOH (0.25 ml, 1.10 mmol) was added to a solution of the carbamate 87 (100 mg, 0.22 mmol) and 50% aqueous hydroxylamine (0.135 mL, 2.20 mmol) in MeOH (5 mL) at 0° C. The reaction mixture was stirred for 1 hr at 0° C., and at room temperature for 1 hr then concentrated. The residue was treated with water and HCl 1N to form a precipitate which was collected by filtration, washed with water and dried. The dry precipitate was purified by Biotage [SNAP 10 g cartridge; 0 to 20% of MeOH (MeOH contained 2% of aqueous ammonia) in DCM over 20 CV]. A solid material was isolated that was further triturated with MeOH to afford the title compound 88 (3.0 mg, 0.006 mmol, 3%) as a white solid. ¹H NMR (400 MHz, DMSO-d₆) δ (ppm): 10.61 (d, J=1.2 Hz, 1H), 8.79 (d, J=1.6 Hz, 1H), 8.48 (s, 1H), 7.28-7.23 (m, 2H), 7.30 (d, J=8.0 Hz, 2H), 7.08 (d, J=8.0 Hz, 2H), 7.00 (d, J=8.0 Hz, 2H), 6.00 (t, J=5.2 Hz, 1H), 3.58 (t, J=4.4 Hz, 4H), 3.21 (s, 2H), 3.18 (q, J=6.4 Hz, 2H), 2.57-2.47 (m, 4H), 2.41-2.34 (m, 4H), 2.36 (t, J=6.4 Hz, 2H), 1.56-1.48 (m, 4H). MS (m/z): 455.3 (M+H).

Compounds 89-91 (examples 48-50) were prepared starting from the compounds 69 or its amino-isomer by following the procedures similar to the ones described above for the synthesis of compound 88 (Scheme 15).

TABLE 11
Characterization of compounds 89-91 (examples 48-50).

| Cpd | Ex. | Structure | Characterization |
|---|---|---|---|
| 89 | 48 | 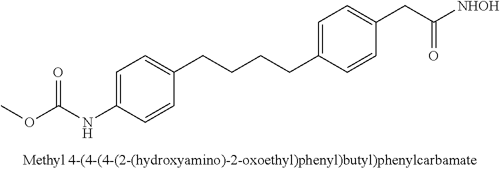 | ¹H NMR (400 MHz, DMSO-d₆) δ (ppm): 10.60 (s, 1H), 9.50 (s, 1H), 8.78 (s, 1H), 7.31 (d, J = 8.0 Hz, 2H), 7.17-7.01 (m, 6H), 3.62 (s, 3H), 3.20 (s, 2H), 2.58-2.51 (m, 4H), 1.57-1.48 (m, 4H). MS (m/z): 357.1 (M + 1). |
| 90 | 49 | 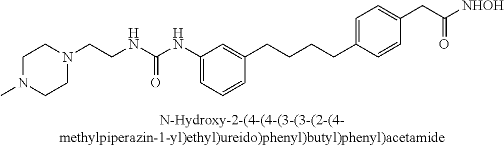 | ¹H NMR (400 MHz, DMSO-d₆) δ (ppm): 10.61 (s, 1H), 8.80 (s, 1H), 8.54 (s, 1H), 7.21-7.02 (m, 7H), 6.68 (d, J = 7.6 Hz, 1H), 6.00 (t, J = 4.4 Hz, 1H), 3.21 (s, 2H), 3.16 (q, J = 6.0 Hz, 2H), 2.62-2.50 (m, 4H), 2.48-2.24 (m, 8H), 2.35 (t, J = 6.0 Hz, 2H), 2.14 (s, 3H), 1.61-1.48 (m, 4H). MS (m/z): 468.4 (M + 1). |
| 91 | 50 | 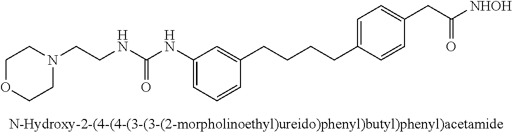 | ¹H NMR (400 MHz, DMSO-d₆) δ (ppm): 10.62 (s, 1H), 8.80 (s, 1H), 8.53 (s, 1H), 7.22-7.03 (m, 7H), 6.68 (d, J = 7.6 Hz, 1H), 6.04 (t, J = 5.6 Hz, 1H), 3.62-3.55 (m, 4H), 3.21 (s, 2H), 3.19 (q, J = 6.0 Hz, 2H), 2.59-2.50 (m, 4H), 2.42-2.34 (m, 6H), 1.60-1.48 (m, 4H). MS (m/z): 455.3 (M + 1). |

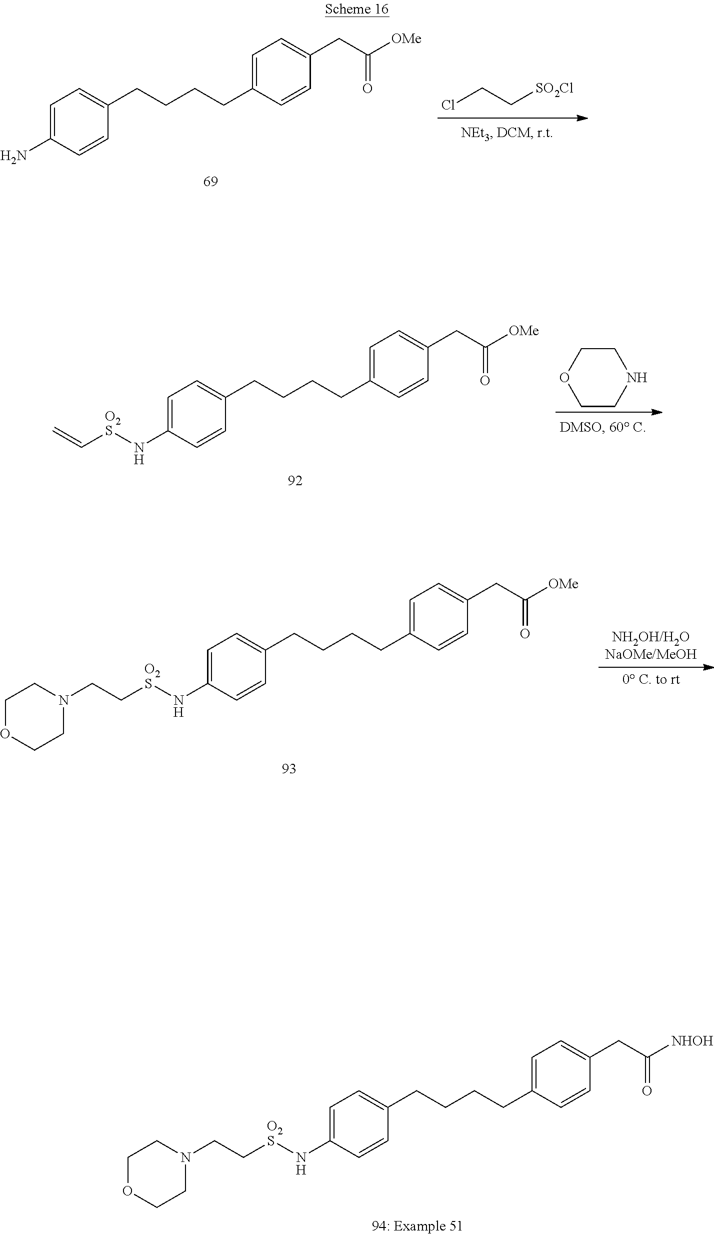

### Example 51

### N-Hydroxy-2-(4-(4-(4-(2-morpholinoethylsulfonamido)phenyl)butyl)phenyl) acetamide (94, Example 51)

### Step 1. Methyl 2-(4-(4-(4-(vinylsulfonamido)phenyl)butyl)phenyl)acetate (92)

2-Chloro-1-ethanesulfonyl chloride (132 μl, 1.26 mmol) was added to a solution of the amine 69 (250 mg, 0.84 mmol) and NEt₃ (352 μl, 2.52 mmol) in DCM (30 mL) and the reaction mixture was stirred for 1.5 hrs before 2-chloro-1-ethanesulfonyl chloride (65 μl, 0.63 mmol) was added. The combined reaction mixture was stirred at room temperature for an additional 1 hr. The reaction was then quenched by addition of water and saturated solution of ammonium chloride and extracted with EtOAc. The organic layer was washed with brine, dried over sodium sulfate, filtered and concentrated. The residue was purified by Biotage (SNAP 25 g cartridge; EtOAc/hex: 0/100 to 30/70 over 20 CV), to afford the title compound 92 (243 mg, 0.63 mmol, 75% yield) as a pinkish solid. ¹H NMR (400 MHz, DMSO-d₆) δ (ppm): 9.83 (s, 1H), 7.18-7.02 (m, 8H), 6.73 (dd, J=10.0 and 16.8 Hz, 1H), 6.05 (d, J=16.8 Hz, 1H), 6.00 (d, J=10.0 Hz, 1H), 3.61 (s, 2H), 3.59 (s, 3H), 2.59-2.51 (m, 4H), 1.58-1.48 (m, 4H).

### Step 2. Methyl methyl 2-(4-(4-(4-(2-morpholinoethylsulfonamido)phenyl)butyl)phenyl)acetate (93)

Morpholine (81 μl, 0.93 mmol) was added to a solution of the vinylsulfonamide 92 (120 mg, 0.31 mmol) and in DMSO (15 mL). The reaction mixture was heated at 60° C. for 20 h. The reaction was quenched by addition of water and saturated solution of ammonium chloride and extracted with EtOAc. The organic layer was washed with water, brine, dried over anhydrous sodium sulfate, filtered and concentrated. The residue was purified by Biotage (SNAP 12 g cartridge; EtOAc/hexanes: 20/80 to 100/0 over 20 CV), to afford the title compound 93 (116 mg, 0.24 mmol, 79% yield) as a colorless oil. MS (m/z): 475.2 (M+H).

Step 3. N-Hydroxy-2-(4-(4-(4-(2-morpholinoethylsulfonamido)phenyl)butyl)phenyl) acetamide (94, Example 51)

Sodium methoxide 25% w/w solution in MeOH (112 μl, 0.52 mmol) was added to a solution of the sulfonamide 93 (116 mg, 0.24 mmol) and 50% aqueous hydroxylamine solution (0.30 mL, 4.88 mmol) in MeOH (5 mL) at 0° C. The reaction mixture was stirred for 1 hr at 0° C. and for 1 hr at room temperature then concentrated. The reaction was quenched by addition of water and HCl 1N, extracted with DCM/MeOH. The organic phases was collected, washed with brine, dried over anhydrous sodium sulfate, filtered and concentrated. The residue was triturated with DCM to afford the title compound 94 (73.5 mg, 0.15 mmol, 63% yield) as a white solid. ¹H NMR (400 MHz, DMSO-d₆) δ (ppm): 10.61 (s, 1H), 9.65 (s, 1H), 8.79 (s, 1H), 7.18-7.07 (m, 8H), 3.50-3.44 (m, 4H), 3.30-3.18 (m, 2H), 3.21 (s, 2H), 2.68-2.61 (m, 2H), 2.59-2.50 (m, 4H), 2.31-2.24 (m, 4H), 1.60-1.48 (m, 4H). MS (m/z): 476.2 (M+H).

Compounds 95-97 (examples 52-53) were prepared starting from the compound 69 or its amino-isomer by following the procedures similar to the ones described above for the synthesis of compound 94 (Scheme 16).

TABLE 12
Characterization of compounds 95-97 (examples 52-54).

| Cpd | Ex. | Structure | Characterization |
|---|---|---|---|
| 95 | 52 | 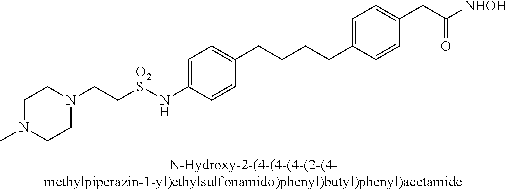 | ¹H NMR (400 MHz, DMSO-d₆) δ (ppm): 10.61 (s, 1H), 9.64 (s, 1H), 8.81 (s, 1H), 7.17-7.05 (m, 8H), 3.21 (s, 2H), 2.21-3.14 (m, 2H), 2.27-2.05 (m, 2H), 2.58-2.50 (m, 4H), 2.38-2.12 (m, 8H), 2.08 (s, 3H), 1.58-1.48 (m, 4H). MS (m/z): 489.2 (M + 1). |
| 96 | 53 | 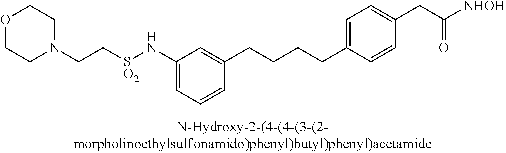 | ¹H NMR (400 MHz, DMSO-d₆) δ (ppm): 10.61 (s, 1H), 9.71 (s, 1H), 8.79 (s, 1H), 7.24-7.19 (m, 1H), 7.14 (d, J = 8.0 Hz, 2H), 7.09 (d, J = 8.0 Hz, 2H), 7.06-7.00 (m, 2H), 6.90 (d, J = 8.0 Hz, 1H), 3.48-3.42 (m, 4H), 3.26-3.22 (m, 2H), 3.21 (s, 2H), 2.67-2.61 (m, 2H), 2.59-2.52 (m, 4H), 2.29-2.22 (m, 4H), 1.60-1.49 (m, 4H). MS (m/z): 476.1 (M + 1). |
| 97 | 54 | 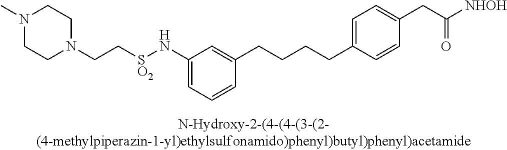 | ¹H NMR (400 MHz, DMSO-d₆) δ (ppm): 10.61 (s, 1H), 7.24-7.19 (m, 1H), 7.14 (d, J = 8.0 Hz, 2H), 7.08 (d, J = 8.0 Hz, 2H), 7.06-7.00 (m, 2H), 6.90 (d, J = 8.0 Hz, 1H), 3.21 (s, 2H), 3.21-3.15 (m, 2H), 2.65-2.60 (m, 2H), 2.59-2.52 (m, 4H), 2.36-2.12 (m, 8H), 2.09 (s, 3H), 1.60-1.49 (m, 4H). MS (m/z): 489.1 (M + 1). |

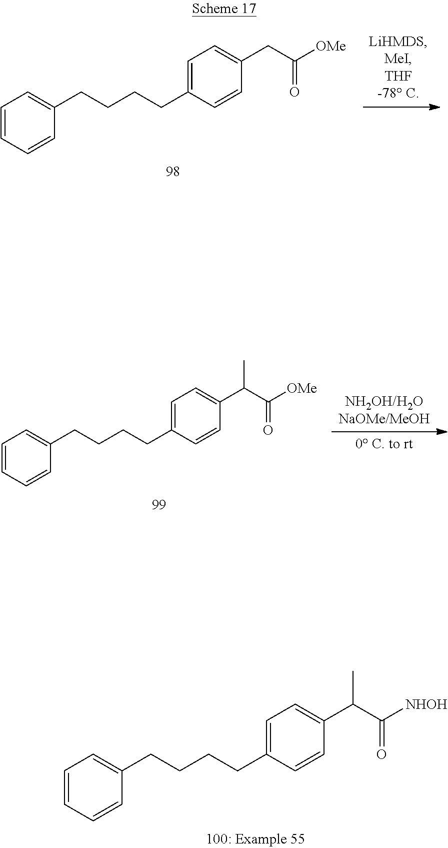

### Example 55

### N-Hydroxy-2-(4-(4-phenylbutyl)phenyl)propanamide (100, Example 55)

### Step 1: Methyl 2-(4-(4-phenylbutyl)phenyl)propanoate (99)

To a solution of methyl 2-(4-(4-phenylbutyl)phenyl)acetate (98) (0.40 g, 1.42 mmol, WO 2008/074132 A1) in THF (40 mL) at −78° C. was added a 1M solution of LiHMDS in toluene (1.70 mL, 1.70 mmol). After 15 min at −78° C., methyl iodide (106 μl, 1.70 mmol) was added and the reaction mixture was stirred for 45 min. The reaction was quenched by addition of water and saturated solution of ammonium chloride and extracted with EtOAc. The organic layer was washed with brine, dried over sodium sulfate, filtered and concentrated. The residue was purified by Biotage (SNAP 25 g cartridge; EtOAc/hex: 0/100 to 10/90 over 20 CV), to afford the title compound 99 (328 mg, 1.11 mmol, 78% yield) as a colorless oil. MS (m/z): 297.1 (M+H).

### Step 2: 2-(4-(4-(4-Aminophenyl)butyl)phenyl)-N-hydroxyacetamide (100, Example 55)

To a solution of the ester 99 (164 mg, 0.55 mmol) and 50% aqueous solution of hydroxylamine (0.678 mL, 11.1 mmol) in MeOH (10 mL) at 0° C. was added 25% w/w sodium methoxide solution in MeOH (1.26 ml, 5.54 mmol). The reaction mixture was stirred for 1 hr at 0° C. and for 1 hr at room temperature then concentrated. The reaction was quenched by addition of water and HCl 1N to form a precipitate that was collected by filtration, washed with water and dried to afford the title compound 100 (123.1 mg, 0.14 mmol, 75% yield) as a white solid. ¹H NMR (400 MHz, DMSO-d₆) δ (ppm): 10.59 (d, J=1.2 Hz, 1H), 8.75 (d, J=1.2 Hz, 1H), 7.30-7.22 (m, 2H), 7.22-7.11 (m, 5H), 7.08 (d, J=8.0 Hz, 2H), 3.37 (q, J=7.2 Hz, 1H), 2.62-2.51 (m, 4H), 1.62-1.50 (m, 4H), 1.29 (d, J=7.2 Hz, 3H). MS (m/z): 298.1 (M+H).

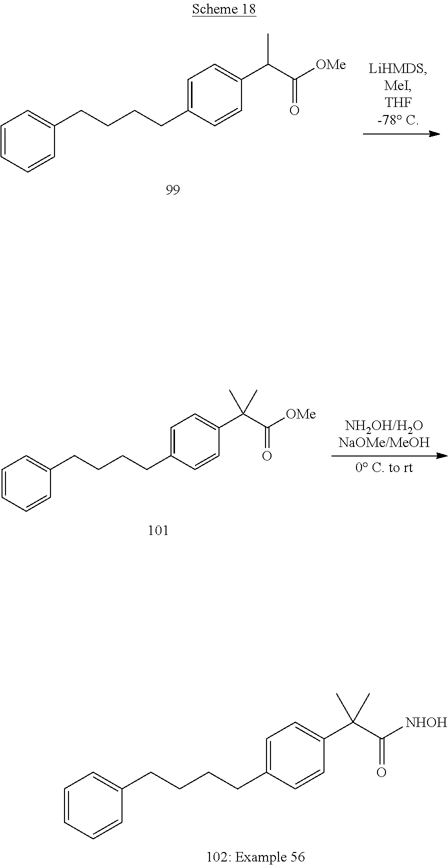

### Example 56

### N-Hydroxy-2-methyl-2-(4-(4-phenylbutyl)phenyl)propanamide (102, Example 56)

### Step 1: Methyl 2-methyl-2-(4-(4-phenylbutyl)phenyl)propanoate (101)

To a solution of the ester 99 (164 mg, 0.55 mmol) in THF (15 mL) at −78° C. was added a 1M solution of LiHMDS in toluene (0.66 mL, 0.66 mmol). After 15 min methyl iodide (42 μl, 0.66 mmol) was added to the reaction mixture at the same temperature. The mixture was allowed to warm to room temperature over 2 hrs. More LiHMDS 1M in toluene (0.66 mL, 0.66 mmol) and methyl iodide (42 μl, 0.66 mmol) were added and the reaction mixture was stirred at room temperature for another 30 min. More LiHMDS 1M in toluene (0.66 mL, 0.66 mmol) and methyl iodide (42 μl, 0.66 mmol) were added and the reaction was stirred at room temperature for an additional 30 min. The reaction was finally quenched by addition of water and saturated solution of ammonium chloride and extracted with EtOAc. The organic layer was washed with brine, dried over anhydrous sodium sulfate, filtered and concentrated. The residue was purified by Biotage (SNAP 25 g cartridge; EtOAc/hexanes: 0/100 to 4/96 over 20 CV), to afford the title compound 101 (130 mg, 0.42 mmol, 76% yield) as a colorless oil. MS (m/z): 297.1 (M+H).

### Step 2: N-Hydroxy-2-methyl-2-(4-(4-phenylbutyl)phenyl)propanamide (102, Example 56)

To a solution of the ester 101 (130 mg, 0.42 mmol) and 50% aqueous hydroxylamine (0.51 mL, 8.38 mmol) in MeOH (10 mL) at 0° C. was added 25% w/w sodium methoxide solution in MeOH (0.96 ml, 4.19 mmol). The reaction mixture was stirred for 1 hr at 0° C. and 1 hr at room temperature then heated at reflux for 1 h. After cooling to room temperature, the reaction mixture was concentrated and diluted with water and HCl 1N. The white precipitate was collected by filtration, washed with water and dried. The dry material was purified by Biotage (SNAP 30 g cartridge KP-C18-HS; MeOH/H₂O: 10/90 to 95/5 over 60 CV), to afford the title compound 102 (18.8 mg, 0.06 mmol, 14% yield) as a white solid. ¹H NMR (400 MHz, DMSO-d₆) δ (ppm): 10.29 (s, 1H), 8.62 (s, 1H), 7.30-7.13 (m, 7H), 7.10 (d, J=8.4 Hz, 2H), 2.59 (t, J=7.2 Hz, 2H), 2.55 (t, J=7.2 Hz, 2H), 1.63-1.51 (m, 4H), 1.41 (s, 6H). MS (m/z): 312.2 (M+H).

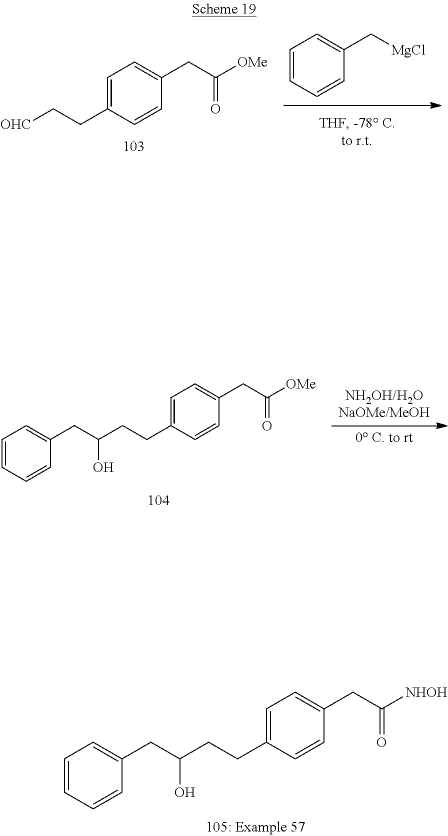

### Example 57

### N-Hydroxy-2-(4-(3-hydroxy-4-phenylbutyl)phenyl)acetamide (105, Example 57)

### Step 1: Methyl 2-(4-(3-hydroxy-4-phenylbutyl)phenyl)acetate (104)

To a solution of methyl 2-(4-(3-oxopropyl)phenyl)acetate acetate (103) (500 mg, 2.42 mmol, WO 2008/074132 A1) in THF (20 mL) at −78° C. was added benzylmagnesium chloride 1M solution in MTBE (2.91 mL, 2.91 mmol). The reaction mixture was stirred at the same temperature for 1 hr at −78° C.; then the mixture was allowed to warm to room temperature over 2 hr period. The reaction was quenched by addition of water and saturated solution of ammonium chloride and extracted with EtOAc. The organic layer was washed with brine, dried over anhydrous sodium sulfate, filtered and concentrated. The residue was purified by Biotage (SNAP 50 g cartridge; EtOAc/hex: 0/100 to 20/80 over 20 CV), to afford the title compound 104 (76 mg, 0.25 mmol, 10% yield) as a colorless oil. MS (m/z): 321.1 (M+Na).

### Step 2: N-Hydroxy-2-methyl-2-(4-(4-phenylbutyl)phenyl)propanamide (105, Example 57)

To a solution of the hydroxy ester 104 (75 mg, 0.25 mmol) and 50% aqueous hydroxylamine solution (0.15 mL, 2.51 mmol) in MeOH (5 mL) at 0° C. was added 25% w/w sodium methoxide solution in MeOH (0.29 ml, 1.26 mmol). The reaction mixture was stirred for 1 hr at 0° C. and 1 hr at room temperature then concentrated. The reaction was quenched by addition of water and HCl 1N to form a precipitate which was collected by filtration, washed with water and dried. The dry material was purified by Biotage (SNAP 30 g cartridge KP-C18-HS; MeOH/H₂O: 10/90 to 95/5 over 40 CV), to afford the title compound 105 (10.6 mg, 0.03 mmol, 14% yield) as a white solid. ¹H NMR (400 MHz, DMSO-d₆) δ (ppm): 10.61 (s, 1H), 8.78 (s, 1H), 7.29-7.22 (m, 2H), 7.21-7.16 (m, 3H), 7.12 (d, J=8.0 Hz, 2H), 7.06 (d, J=8.0 Hz, 2H), 4.63 (d, J=5.6 Hz, 1H), 3.68-3.59 (m, 1H), 3.21 (s, 2H), 2.74-2.66 (m, 1H), 2.66 (d, J=6.4 Hz, 2H), 2.58-2.52 (m, 1H), 1.68-1.48 (m, 2H). MS (m/z): 300.1 (M+H).

Compound 106 (example 58) was prepared starting from the compound 105 by following the procedures similar to the ones described above for the synthesis of compound 100 (Scheme 17). Compound 107 (example 59) was prepared starting from methyl 2-(4-formylphenyl)acetate and (3-phenylpropyl)magnesium chloride by following the procedures similar to the ones described above for the synthesis of compound 105 (Scheme 19).

TABLE 13
Characterization of compounds 106-107 (examples 58-59).

| Cpd | Ex. | Structure | Characterization |
|---|---|---|---|
| 106 | 58 | 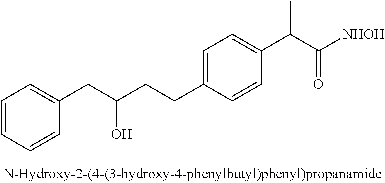 | ¹H NMR (400 MHz, DMSO-d₆) δ (ppm): 10.58 (s, 1H), 8.74 (s, 1H), 7.28-7.22 (m, 2H), 7.22-7.12 (m, 5H), 7.10-7.04 (m, 2H), 4.63 (d, J = 6.0 Hz, 1H), 3.68-3.49 (m, 1H), 3.36 (q, J = 7.2 Hz, 1H), 2.73-2.65 (m, 1H), 2.66 (d, J = 6.0 Hz, 2H), 1.68-1.48 (m, 2H), 1.29 (d, J = 7.2 Hz, 3H). MS (m/z): 314.1 (M + 1). |
| 107 | 59 | 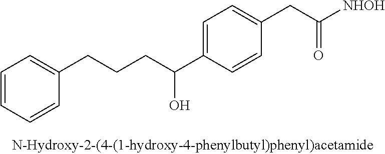 | ¹H NMR (400 MHz, DMSO-d₆) δ (ppm): 10.62 (s, 1H), 8.79 (s, 1H), 7.30-7.11 (m, 9H), 5.09 (d, J = 4.0 Hz, 1H), 4.53-4.46 (m, 1H), 3.23 (s, 2H), 2.59-2.52 (m, 2H), 1.68-1.46 (m, 4H). MS (m/z): 300.1 (M + 1). |

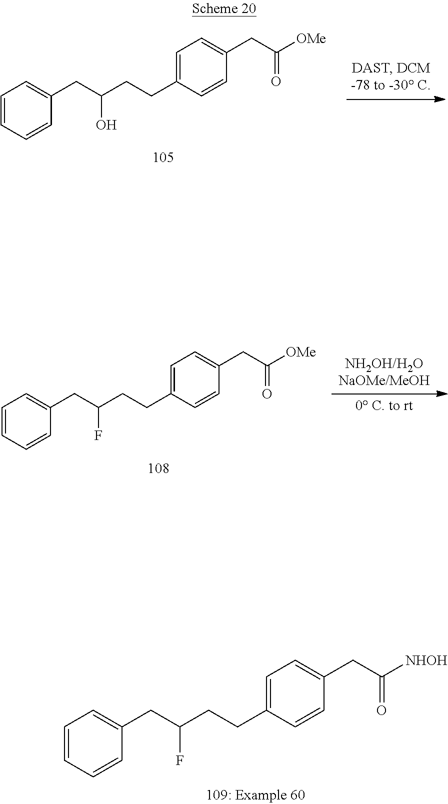

### Example 60

### 2-(4-(3-Fluoro-4-phenylbutyl)phenyl)-N-hydroxyacetamide (109, Example 60)

### Step 1: Methyl 2-(4-(3-fluoro-4-phenylbutyl)phenyl)acetate (108)

To a solution of the ester 105 (75 mg, 0.25 mmol) in DCM (10 mL) at −78° C. was added DAST (43 μl, 0.33 mmol). The reaction mixture was stirred for 1 h at −78° C. before more DAST (22 μl, 0.16 mmol) was added. After 30 min of stirring, the reaction mixture was warmed to −30° C. over 30 min. The reaction was quenched by addition of water and saturated solution of ammonium chloride, and extracted with DCM. The organic layer was washed with brine, dried over anhydrous sodium sulfate, filtered and concentrated. The residue was purified by Biotage (SNAP 25 g cartridge; EtOAc/hex: 0/100 to 20/80 over 20 CV), to afford the title compound 108 (58 mg, 0.19 mmol, 77% yield) as a light yellow solid. MS (m/z): 323.1 (M+Na).

### Step 2: 2-(4-(3-Fluoro-4-phenylbutyl)phenyl)-N-hydroxyacetamide (109, Example 60)

To a solution of the ester 108 (58 mg, 0.19 mmol) and 50% aqueous hydroxylamine (0.12 mL, 1.93 mmol) in MeOH (5 mL) at 0° C. was added 25% w/w sodium methoxide in MeOH (0.22 ml, 0.96 mmol). The reaction mixture was stirred for 1 hr at 0° C., for 1 hr at room temperature then concentrated. The reaction was quenched by addition of water and HCl 1N to form a precipitate that was collected by filtration washed with water and dried. The dry material was purified by Biotage (SNAP 30 g cartridge KP-C18-HS; MeOH/H₂O: 10/90 to 95/5 over 60 CV), to afford the title compound 109 (30.3 mg, 0.10 mmol, 52% yield) as a white solid. ¹H NMR (400 MHz, DMSO-d₆) δ (ppm): 10.61 (s, 1H), 8.79 (s, 1H), 7.33-7.26 (m, 2H), 7.26-7.18 (m, 3H), 7.15 (d, J=7.6 Hz, 2H), 7.11 (d, J=7.6 Hz, 2H), 4.75 and 4.63 (m, J=49.2 Hz, 1H), 3.22 (s, 2H), 3.02-2.83 (m, 2H), 2.78-2.56 (m, 2H), 1.92-1.78 (m, 2H). MS (m/z): 302.1 (M+H).

Compound 110 (example 61) was prepared by following the procedures similar to the ones described above for the synthesis of compounds 107 (table 9) and 109 (Scheme 20).

TABLE 14
Characterization of compound 110 (example 61).

| Cpd | Ex. | Structure | Characterization |
|---|---|---|---|
| 110 | 61 | 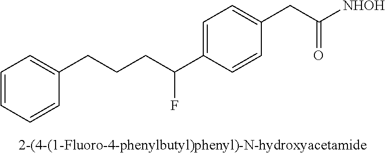 | ¹H NMR (400 MHz, DMSO-d₆) δ (ppm): 10.65 (s, 1H), 8.83 (s, 1H), 7.30-7.23 (m, 6H), 7.20-7.14 (m, 3H), 5.51 (ddd, J = 4.8, 8.0 and 48.0 Hz, 1H), 3.27 (s, 2H), 2.61 (t, J = 7.2 Hz, 2H), 1.99-1.52 (m, 4H). MS (m/z): 302.2 (M + 1). |

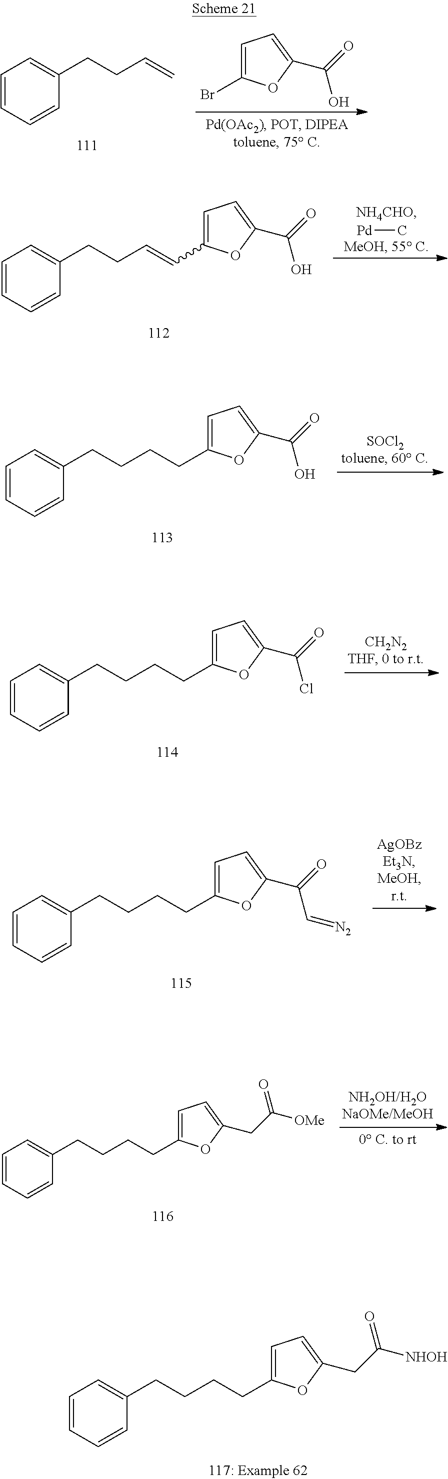

### Example 62

### N-Hydroxy-2-(5-(4-phenylbutyl)furan-2-yl)acetamide (117, Example 62)

### Step 1: 5-(4-Phenylbut-1-enyl)furan-2-carboxylic acid (112)

To a degassed suspension of 4-phenyl-1-butene 111 (4.15 g, 31.4 mmol), 5-bromo-2-furoic acid (3 g, 15.71 mmol), Pd(OAc)₂ (176 mg, 0.78 mmol) and POT (478 mg, 1.57 mmol) in toluene (30 mL) was added DIPEA (5.49 mL, 31.4 mmol). The reaction mixture was heated at 75° C. for 3.5 h. After cooling to room temperature, the reaction was quenched by addition of water and 5% HCl. The organic layer was separated and the aqueous layer was discarded. The organic layer was then extracted with 1M NaOH solution and the basic extract was acidified with 10% HCl solution. The acidified solution was extracted with EtOAc. The organic extract was washed with water and brine, dried over anhydrous sodium sulfate, filtered and concentrated. The residue was purified by Biotage (SNAP 50 g cartridge; EtOAc/hex: 40/60 to 100/0 over 20 CV), to afford compound 112 (3.66 g, 15.1 mmol, 96% yield, mixture of E- and Z-isomers) as a yellow oil. MS (m/z): 242.9 (M+H).

### Step 2: 5-(4-Phenylbutyl)furan-2-carboxylic acid (113)

Ammonium formate (2.38 g, 37.8 mmol) was added to a suspension of the acid 112 (3.66 g, 15.1 mmol) and Pd—C 10% Degussa type (1.28 g, 1.21 mmol) in MeOH (40 mL). The reaction mixture was stirred at 55° C. for 20 hrs. More ammonium formate (2.38 g, 37.8 mmol) was added and the reaction mixture was stirred at 55° C. for an additional 2 h then concentrated. The residue was diluted with 2N HCl solution and extracted with EtOAc. The organic layer was washed with brine, dried over anhydrous sodium sulfate, filtered and concentrated. The remained material was purified by Biotage (SNAP 25 g; EtOAc/Hex: 0/100 to 10/60 over 20 CV), to afford the title compound 113 (2.1 g, 8.60 mmol, 57% yield) as a light yellow solid. MS (m/z): 245.2 (M+H).

### Step 3: 5-(4-Phenylbutyl)furan-2-carbonyl chloride (114)

SOCl₂ (2.52 mL, 34.5 mmol) was added to a solution of the acid 113 (2.1 g, 8.64 mmol) in toluene (20 mL). The reaction mixture was heated at 60° C. for 4 hr and concentrated. The resulting material (114, 2.09 g, 7.95 mmol, 92% yield) was used as is in the next step.

### Step 4: 2-Diazo-1-(5-(4-phenylbutyl)furan-2-yl)ethanone (115)

A 1.1 M solution of diazomethane in Et₂O (18.08 mL, 19.89 mmol) was added to a solution of carbonyl chloride 114 (2.09 g, 7.95 mmol) in THF (20 mL) at 0° C. The reaction mixture was stirred at 0° C. for 5 min then at room temperature for 10 min. More 1.1 M solution of diazomethane in Et₂O (18.08 mL, 19.89 mmol) was added and the reaction mixture was stirred at room temperature for and additional 20 hrs then concentrated. The residue was purified by Biotage (SNAP 100 g; EtOAc/Hex: 0/100 to 20/80 over 20 CV), to afford the title compound 115 (194 mg, 0.72 mmol, 9% yield) as a brown oil. MS (m/z): 269.3 (M+H).

### Step 5: Methyl 2-(5-(4-phenylbutyl)furan-2-yl)acetate (116)

A solution of silver benzoate (93 mg, 0.40 mmol) in Et₃N (4 ml) was added to a solution of the diazo-compound 115 (194 mg, 0.72 mmol) in MeOH (15 mL). The reaction mixture was stirred at room temperature for 3.5 hrs then concentrated. The residue was purified by Biotage (SNAP 25 g; EtOAc/Hex: 0/100 to 20/80 over 20 CV), to afford the title compound 116 (56 mg, 0.21 mmol, 28% yield) as a yellow solid. ¹H NMR (400 MHz, DMSO-d₆) δ (ppm): 7.30-7.14 (m, 5H), 6.09 (d, J=3.2 Hz, 1H), 5.90 (d, J=3.2 Hz, 1H), 3.71 (s, 3H), 3.64 (s, 2H), 2.67-2.58 (m, 4H), 1.70-1.63 (m, 4H).

### Step 6: N-Hydroxy-2-(5-(4-phenylbutyl)furan-2-yl)acetamide (117, Example 62)

To a solution of the ester 116 (56 mg, 0.21 mmol) and 50% aqueous hydroxylamine (63 μl, 2.05 mmol) in MeOH (5 mL) at 0° C. was added 25% w/w sodium methoxide in MeOH (0.23 ml, 1.03 mmol). The reaction mixture was stirred for 1 h at 0° C., and for 1 hr at room temperature then concentrated. The reaction was quenched by addition of water and HCl 1N to form a precipitate that was collected by filtration, washed with water and dried over anhydrous sodium sulfate. The dry material was purified by Biotage (SNAP 10 g; MeOH/DCM: 0/100 to 15/85 over 20 CV), to afford the title compound 117 (6.4 mg, 0.02 mmol, 11% yield) as a light beige solid. ¹H NMR (400 MHz, DMSO-d₆) δ (ppm): 10.55 (bs, 1H), 8.85 (bs, 1H), 7.30-7.22 (m, 2H), 7.20-7.13 (m, 3H), 6.04 (d, J=2.8 Hz, 1H), 5.95 (m, J=2.8 Hz, 1H), 3.21 (s, 2H), 2.62-2.53 (m, 4H), 1.66-1.51 (m, 4H). MS (m/z): 274.1 (M+H).

Compound 118 (example 63) was obtained by following the procedures similar to the ones described above for the synthesis of compound 27 (Scheme 5) using 3-cyclopentylpropanal instead of 3-cyclohexylpropanal (23). Compounds 119 and 120 (examples 64-65) were prepared in three steps by following the procedures similar to the ones described above for the synthesis of compound 15 (Scheme 3) starting from compound 12 and replacing the 2,4-difluoro-1-iodobenzene with the 2-iodo- or 3-iodothiophene, respectively.

TABLE 15
Characterization of compounds 118-120 (examples 63-65).

| Cpd | Ex. | Structure | Characterization |
|---|---|---|---|
| 118 | 63 | 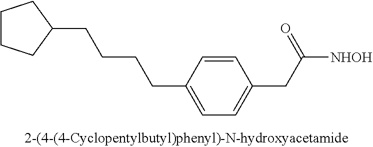 | ¹H NMR (400 MHz, DMSO-d₆) δ (ppm): NH— and OH— signals are not observed; 7.15 (d, J = 8.2 Hz, 2H), 7.09 (d, J = 8.0 Hz, 2H), 3.22 (s, 2H), 2.52 (t, partially overlapped with the residual solvent signal, J = 7.6 Hz, 2H), 1.72-1.67 (m, 3H), 1.57-1.44 (m, 6H), 1.30-1.28 (m, 4H), 1.04-1.02 (m, 2H); MS (m/z): 276.1 (M + 1). |
| 119 | 64 | 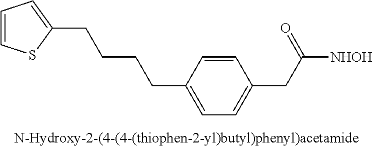 | ¹H NMR (400 MHz, DMSO-d₆) δ (ppm): NH— and OH— signals are not observed; 7.28 (dd, J = 1.2 and 6.1 Hz, 1H), 7.15 (d, J = 8.0 Hz, 2H), 7.09 (d, J = 8.2 Hz, 2H), 6.91 (dd, J = 3.3 and 5.1 Hz, 1H), 6.83- 6.81 (m, 1H), 3.22 (s, 2H), 2.81 (t, J = 6.7 Hz, 2H), 2.57 (t, J = 7.0 Hz, 2H), 1.60 (t, J = 3.5 Hz, 4H); MS (m/z): 290.2 (M + 1). |
| 120 | 65 | 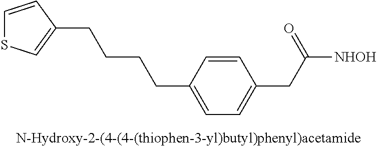 | ¹H NMR (400 MHz, DMSO-d₆) δ (ppm): 10.60 (bs, 1H), 8.78 (bs, 1H), 7.42 (dd, J = 2.9 and 4.9 Hz, 1H), 7.16-7.08 (m, 5H), 6.82 (dd, J = 1.2 and 4.9 Hz, 1H), 3.22 (s, 2H), 2.60 (t, J = 7.0 Hz, 2H), 2.56 (t, J = 7.2 Hz, 2H), 1.57 (t, J = 3.5 Hz, 4H); MS (m/z): 290.1 (M + 1). |

### ro Assay Examples

### Activity of Example Compounds Against Fungi and Yeasts

The data presented herein demonstrate the antifungal agent potentiating effect of the compounds of the invention. These data lead one to reasonably expect that the compounds of the present invention are useful for not only potentiating the effect of an antifungal agent, but also as therapeutic agents for the treatment of a fungal infection, including infection by species such as Candida spp. and Aspergillus spp.

The in vitro activities of the inventive compounds (compound(s) herein) against fungi and/or yeasts were determined with isolates Candida albicans, Candida glabrata and Candida parapsilosis. The comparator compounds included fluconazole, caspofungin and amphotericin B.

Compound Preparation—

The compounds were supplied as a powder, and were dissolved in dimethylsulfoxide (DMSO) the day of assay. Stock solutions were prepared at 10 mg/mL and diluted to a starting stock concentration of 2.56 mg/mL. Amphotericin B was dissolved in DMSO to a stock concentration of 2.56 mg/mL. Fluconazole was dissolved in deionized water to a stock concentration of 2.56 mg/mL. Caspofungin was received as a stock solution of 3.2 mg/mL in DMSO and was diluted to a starting stock concentration of 2.56 mg/mL.

Test Organisms—

The test organisms and reference isolates were streaked for isolation on Sabouraud agar medium (yeasts) or potato dextrose agar (fungi). Colonies were picked by swab from the medium and resuspended in the appropriate broth containing cryoprotectant. The suspensions were aliquoted into cryogenic vials and maintained at −80° C.

Prior to testing, the yeast isolates were streaked from the frozen vials onto Sabouraud dextrose agar. The plates were incubated overnight at 35° C. The fungal isolate was grown on potato dextrose agar slants and was incubated for 7-10 days at room temperature.

Test Medium—

Both yeast and fungal isolates were tested in RPMI medium buffered with 0.165 M MOPS (3-(N-morpholino)propanesulfonic acid). The pH of the medium was adjusted to 7.0 with 1 N NaOH. The medium was sterile filtered using a 0.2 um PES (polyethersulfone) filter and stored at 4° C. until used.

Minimal Inhibitory Concentration (MIC) Assay Procedure—

The MIC assay method followed the procedure described by the Clinical and Laboratory Standards Institute (CLSI; 1, 2, 3) and employed automated liquid handlers to conduct serial dilutions and liquid transfers. Automated liquid handlers included the Multidrop 384 (Labsystems, Helsinki, Finland), Biomek 2000 and Biomek FX (Beckman Coulter, Fullerton Calif.). The wells in columns 2-12 in standard 96-well microdilution plates (Costar 3795) were filled with 150 ul of 100% DMSO. These would become the ‘mother plates’ from which ‘daughter’ or test plates would be prepared. The compounds (300 uL at 40× the desired top concentration in the test plates) were dispensed into the appropriate well in Column 1 of the mother plates. The Biomek 2000 was used to make serial 1:1 dilutions through Column 11 in the “mother plate”. The wells of Column 12 contained no compounds and were the organism growth control wells.

The daughter plates were loaded with 185 uL per well of RPMI described above using the Multidrop 384. The daughter plates were prepared using the Biomek FX which transferred 5 μL of the compound solution from each well of a mother plate to the corresponding well of the daughter plate in a single step.

Standardized inoculum of each organism was prepared per CLSI methods. For yeast isolates, colonies were picked from the streak plate and a suspension was prepared in 0.85% saline. For the fungal isolate, 1 mL of 0.85% saline was dispensed onto the potato dextrose agar slant that had been inoculated 7-10 days previously. Using a swab, a suspension of the fungus was made. After a short time to allow the heavy particles to settle out, a small quantity of the supernatant was dispensed into 0.85% saline and the suspension adjusted to equal a 0.5 McFarland turbidity standard. Both yeast and fungal isolates were diluted 1:100 in RPMI and then transferred to compartments of sterile reservoirs divided by length (Beckman Coulter). The Biomek 2000 was used to inoculate the plates. Daughter plates were placed on the Biomek 2000 work surface reversed so that inoculation took place from low to high compound concentration. The Biomek 2000 delivered 10 μL of standardized inoculum into each well. Thus, the wells of the daughter plates ultimately contained 185 uL of RPMI, 5 uL of the compound solution, and 10 uL of inoculum. For the assay, each well had a final concentration of 2.5% DMSO.

Plates were stacked three high, covered with a lid on the top plate, placed in plastic bags, and incubated at 35° C. for approximately 24-48 hr prior to reading. The microplates were viewed from the bottom using a plate viewer. An un-inoculated solubility control plate was observed for evidence of compound precipitation. The MIC was read and recorded as the lowest concentration of compound that inhibited visible growth of the organism. Per CLSI (1), fluconazole MICs for yeast isolates were recorded where a prominent decrease in visible growth was observed.

For the fungal isolate, both a Minimal Inhibitory Concentration (MIC) and Minimal Effective Concentration (MEC) value were recorded. The MEC value is applied specifically to echinocandins when testing filamentous fungi (CLSI; 3). While the MIC value is the amount of compound that inhibits visible growth of the organism, the MEC value is the lowest concentration of compoundthat leads to the growth of small, rounded, compact hyphal forms as compared to the hyphal growth seen in the growth control well. MEC values, which typically differ from MIC values for this class of antifungal agents, are the measure that should be used for determining susceptibility to echinocandins. MIC and MEC values for all test compounds were reported. MEC and MIC values were also reported for caspofungin. MECs were not reported for fluconazole or amphotericin B, as the MEC reading does not apply to these agents.

TABLE 16
Minimum Inhibitory Concentrations (ug/mL) of Example Compounds
against Yeast Isolates (24 hr/48 hr).

| Compound | Candida | | Candida |
|---|---|---|---|
| Example # | albicans | Candida parapsilosis | glabrata |
| 1 | 32/32 | 32/32 | >64/>64 |
| 2 | 64/64 | 64/64 | >64/>64 |
| 3 | 0.5/0.5 | 0.25/0.5 | 0.5/0.5 |
| 4 | 0.25/0.25 | 0.12/0.25 | 0.25/0.25 |
| 5 | >8/>8 | >8/>8 | 0.5/8 |
| 6 | 0.5/0.5 | >4>4 | 0.25/0.5 |
| 7 | >8/>8 | >8/>8 | 0.5/>8 |
| 8 | 0.25/0.25 | 0.12/0.25 | 0.12/0.12 |
| 9 | 1/1 | 1/1 | 0.5/>4 |
| 10 | >64/>64 | >64/>64 | >64/>64 |
| 11 | >64/>64 | >64/>64 | >64/>64 |
| 12 | >64/>64 | >64/>64 | 4/16 |
| 13 | 8/ 16 | 16/>16 | 2/4 |
| 14 | 16/>64 | 32/>64 | 32/64 |
| 15 | >16/>16 | >16/>16 | >8/>8 |
| 16 | >16/>16 | >16/>16 | >32/>32 |
| 17 | >64/>64 | >64/>64 | >64/>64 |
| 18 | >64/>64 | >64/>64 | >64/>64 |
| 19 | 16/32 | 32/32 | 1/2 |
| 20 | >8/>8 | >8/>8 | 2/2 |
| 21 | >64/>64 | >64/>64 | 16/32 |
| 22 | >8/>8 | >8/>8 | 8/>8 |
| 23 | >8/>8 | >8/>8 | 8/>8 |
| 24 | >64/>64 | >64/>64 | 16/64 |
| 25 | >64/>64 | >64/>64 | 16/64 |
| 26 | 4/8 | 4/8 | 2/8 |
| 27 | 64/64 | >64/>64 | 8/16 |
| 28 | 32/32 | >32/>32 | 8/16 |
| 29 | 1/2 | 1/2 | 0.5/1 |
| 30 | 0.5/0.5 | 0.12/0.25 | 0.25/0.5 |
| 31 | >16/>16 | >16/>16 | >8/>8 |
| 32 | 32/32 | 32/32 | >32/>32 |
| 33 | 8/16 | 8/16 | 4/8 |
| 34 | >64/>64 | >64/>64 | >64/>64 |
| 35 | 64/64 | 64/64 | 64/>64 |
| 36 | >8/>8 | >8/>8 | >8/>8 |
| 37 | >64/>64 | >64/>64 | 64/>64 |
| 38 | >64/>64 | >64/>64 | 16/16 |
| 39 | >8/>8 | >8/>8 | >8/>8 |
| 40 | >64/>64 | >64/>64 | 2/32 |
| 41 | >32/>32 | >32/>32 | 4/16 |
| 42 | >32/>32 | >32/>32 | 8/16 |
| 43 | >64/>64 | >64/>64 | 16/64 |
| 44 | >64/>64 | 64/>64 | 16/64 |
| 45 | >64/>64 | >64/>64 | 32/>64 |
| 46 | >64/>64 | >64/>64 | 64/>64 |
| 47 | >32/>32 | >32/>32 | 4/16 |
| 48 | >4/>4 | >4/>4 | >4/>4 |
| 49 | >64/>64 | >64/>64 | 16/>64 |
| 50 | >64/>64 | >64/>64 | 8/16 |
| 51 | >64/>64 | >64/>64 | 4/16 |
| 52 | >64/>64 | >64/>64 | 8/>64 |
| 53 | >64/>64 | >64/>64 | 16/32 |
| 54 | >64/>64 | >64/>64 | 16/>64 |
| 55 | 2/4 | 0.5/1 | 0.25/0.5 |
| 56 | >2/>2 | >2/>2 | 0.25/0.5 |
| 57 | >64/>64 | >64/>64 | 16/>64 |
| 58 | >64/>64 | >64/>64 | 8/64 |
| 59 | >64/>64 | >64/>64 | 16/64 |
| 60 | 4/4 | 4/8 | 2/2 |
| 61 | 4/8 | 8/16 | 2/4 |
| 62 | 32/32 | 32/32 | 32/32 |
| Fluconazole | 0.25/1 | 2/4 | 4/8 |
| | (NA/0.25-1) | (0.5-4/1-4) | |
| Caspofungin | 0.06/0.06 | 0.5/0.5 | 0.12/0.12 |
| | | (0.25-1/0.5/4) | |
| Amphotericin B | <0.06/0.25 | 0.25/0.5 | <0.06/0.5 |

In Vitro Activity of Example Compounds in Combination with Azoles

The ability of the compounds to synergize with azoles against C. glabrata was evaluated by the checkerboard assay, a common laboratory method used to evaluate synergy, antagonism, and indifference using fractional inhibitory concentrations (FICs) and FIC indices (FICI). The sub-inhibitory concentrations of the compounds exhibit sufficient synergy with fluconazole to result in fluconazole MICs typically associated with susceptible isolates (<8 ug/mL) for isolates where fluconazole alone had MICs>64 ug/mL.

Compound Preparation—

The test compounds were supplied as a powder and dissolved in dimethylsulfoxide (DMSO) on the day of assay. Stock solutions were prepared at 640 ug/mL. Fluconazole was dissolved in deionized water to a stock concentration of 320 ug/mL. All test articles were in solution under these conditions.

Test Organisms—

The test organisms and reference isolates were streaked for isolation on Sabouraud agar medium (yeasts) or potato dextrose agar (fungi). Colonies were picked by swab from the medium and resuspended in the appropriate broth containing cryoprotectant. The suspensions were aliquoted into cryogenic vials and maintained at −80° C.

Prior to testing, the yeast isolates were streaked from the frozen vials onto Sabouraud dextrose agar. The plates were incubated overnight at 35° C. The fungal isolate was grown on potato dextrose agar slants and was incubated for 7-10 days at room temperature.

Test Medium—

Both yeast and fungal isolates were tested in RPMI medium buffered with 0.165 M MOPS (3-(N-morpholino)propanesulfonic acid). The pH of the medium was adjusted to 7.0 with 1 N NaOH. The medium was sterile filtered using a 0.2 um PES (polyethersulfone) filter and stored at 4° C. until used.

FIC Assay Methodology —

FIC values were determined using a broth microdilution method (CLSI; 1, 3). To prepare the test plates, automated liquid handlers (Multidrop 384, Labsystems, Helsinki, Finland; Biomek 2000 and Biomek FX, Beckman Coulter, Fullerton Calif.) were used to conduct serial dilutions and liquid transfers.

The wells of standard 96-well microdilution plates (Costar 3795) were filled with 150 uL of DMSO in columns 2-12. Three hundred microliters of each test compound was added to each well in Column 1 of the plates. The Biomek 2000 was used to make eleven 2-fold serial dilutions in each of these “test compound mother” plates. The wells of the “fluconazole mother plates” were filled with 150 uL of sterile water in rows B-H. Row A of these plates was filled with 300 uL of the fluconazole stock solution. Serial 2-fold dilutions were then prepared from top to bottom by hand, using a multichannel pipette in rows B-G. The “daughter plates” were loaded with 180 uL of RPMI using the Multidrop 384. The Biomek FX was used to transfer 5 uL of compound solution from each well of a fluconazole mother plate to the corresponding well in all of the daughter plates in a single step. Then the Biomek FX was used to transfer 5 uL of compound solution from each well of a test compound mother plate to the corresponding well of the corresponding test compound daughter plate (already containing fluconazole at the appropriate concentrations) in a single step. Row H and Column 12 each contained serial dilutions of the test agent and fluconazole alone, respectively, for determination of the MIC.

Standardized inoculum of each organism was prepared per CLSI methods (CLSI; 1, 3). For yeast isolates, colonies were picked from the primary plate and a suspension was prepared in 0.85% saline to equal a 0.5 McFarland turbidity standard. For the fungal isolate, 1 mL of 0.85% saline was dispensed onto a potato dextrose agar slant onto which the isolate had been inoculated 7-10 days previously. Using a swab, a suspension of the fungus was made. After a short time to allow the heavy particles to settle out, a small quantity of the supernatant was dispensed into 0.85% saline and the suspension was adjusted to equal a 0.5 McFarland turbidity standard. Both yeast and fungal isolates were diluted 1:100 in RPMI and then transferred to compartments of sterile reservoirs divided by length (Beckman Coulter). The Biomek 2000 was used to inoculate the plates. Daughter plates were placed on the Biomek 2000 in reverse orientation so that plates were inoculated from low to high drug concentration. The Biomek 2000 delivered 10 uL of standardized inoculum into each well. Thus, the wells of the daughter plates ultimately contained 180 uL of RPMI, 10 uL of compound solutions, and 10 uL of inoculum. The test format resulted in the creation of an 8×12 checkerboard where each compound was tested alone and in combination at varying ratios of compound concentration.

All organism plates were stacked three high, covered with a lid on the top plate, placed in plastic bags, and incubated at 35° C. for approximately 48 hr. Following incubation, the microplates were removed from the incubators and viewed from the bottom using a ScienceWare plate viewer. Prepared reading sheets were marked for the MIC of fluconazole (column 12), the MIC of the test agent (row H) and the wells of the growth-no growth interface for wells containing combinations of fluconazole and the test agent. An un-inoculated solubility control plate was observed for evidence of compound precipitation. The MIC was read and recorded as the lowest concentration of compound that exhibited a prominent decrease in visible growth of the organism.

FIC/FICI Calculations —

FIC indices (FICI) according to the formula: FIC_{fluconazole} (MIC of fluconazole in combination/MIC of fluconazole alone)+FIC_{testagent} (MIC of test agent in combination/MIC of test agent alone). In instances where an agent alone yielded an off-scale MIC result, the next highest concentration was used as the MIC value in the FIC calculation. In this study, fluconazole had an MIC of >8 μg/mL against both test isolates. As a result, an MIC of 16 μg/mL was used for FIC calculations. Based on a prior study (1), the MIC of fluconazole against both test isolates was >64 μg/mL, so additional FICI values were determined using a fluconazole MIC of 128 μg/mL for FIC calculations.

FICI values have been interpreted in a variety of ways (4, 5). In this study, FICI values have been interpreted as follows: <0.50, synergy; >0.50-4.0, indifference; >4.0, antagonism (5). An interpretation of “synergy” is consistent with inhibition of organism growth by combinations at concentrations significantly below (>4-fold) the MIC of either compound alone, resulting in a low FICI value (<0.50). An interpretation of “indifference” is consistent with growth inhibition at concentrations at or slightly below/above the MICs of the individual compounds alone, resulting in an FICI value of >0.50 but less than or equal to 4.0. An interpretation of “antagonism” results when the concentrations of the compounds in combination that are required to inhibit organism growth are substantially greater (>4-fold) than those for the compounds individually, resulting in an FICI value of >4.0.

TABLE 17
Summary of in vitro Activity of Example Compounds in Combination
with Fluconazole Against C. glabrata: MIC of Compounds Alone
and in Combination.

| Com- | | Concentration of Methylgene compound | | | | | |
|---|---|---|---|---|---|---|---|
| pound | Comppuond | (ug/mL; [multiple of MIC]) | | | | | |
| Exam- | MIC | to synergize fluconazole MIC to: | | | | | |
| ple # | (ug/mL) | 8 ug/mL | | 4 ug/mL | | 2 ug/mL | |
| 1 | 8 | 1 | (⅛x) | 2 | (¼x) | 4 | (½x) |
| 2 | 16 | 2 | (⅛x) | 4 | (¼x) | 4 | (¼x) |
| 3 | 0.5 | 0.12 | (¼x) | 0.25 | (½x) | 0.25 | (½x) |
| 4 | 0.25 | 0.06 | (¼x) | 0.12 | (½x) | 0.25 | (1x) |
| 5 | 4 | 0.12 | ( 1/16x) | 0.12 | ( 1/16x) | 0.25 | ( 1/16x) |
| 6 | 1 | 0.12 | (⅛x) | 0.12 | (⅛x) | 0.25 | (¼x) |
| 7 | >4 | 0.25 | (NA) | 0.5 | (NA) | 0.5 | (NA) |
| 8 | 0.25 | 0.12 | (½x) | 0.12 | (½x) | 0.12 | (½x) |
| 9 | 0.5 | 0.06 | (⅛x) | 0.12 | (¼x) | 0.25 | (½x) |
| 10 | 2 | 0.25 | (⅛x) | 0.5 | (¼x) | 0.5 | (¼x) |
| 11 | 8 | 1 | (⅛x) | 1 | (⅛x) | 2 | (¼x) |
| 12 | 16 | 2 | (⅛x) | 2 | (⅛x) | 4 | (¼x) |
| 13 | 4 | 0.5 | (⅛x) | 1 | (¼x) | 1 | (¼x) |
| 14 | >16 | 16 | (NA) | 16 | (NA) | 16 | (NA) |
| 15 | 8 | 1 | (⅛x) | 2 | (¼x) | 2 | (¼x) |
| 16 | 2 | 0.25 | (⅛x) | 0.5 | (¼x) | 0.5 | (¼x) |
| 19 | 2 | 0.5 | (¼x) | 0.5 | (¼x) | 0.5 | (¼x) |
| 20 | 1 | 0.25 | (¼x) | 0.25 | (¼x) | 0.5 | (½x) |
| 21 | 16 | 2 | (⅛X) | 2 | (⅛X) | 4 | (¼x) |
| 22 | 16 | 1 | ( 1/16x) | 2 | (⅛x) | 2 | (⅛x) |
| 23 | >8 | 2 | (NA) | 2 | (NA) | 4 | (NA) |
| 24 | >16 | 2 | (NA) | 4 | (NA) | 8 | (NA) |
| 25 | >16 | 4 | (NA) | 8 | (NA) | 16 | (NA) |
| 26 | 8 | 0.5 | ( 1/16x) | 1 | (⅛x) | 2 | (¼x) |
| 27 | 16 | 2 | (⅛x) | 4 | (¼x) | 4 | (¼x) |
| 28 | 16 | 1 | ( 1/16x) | 2 | (⅛x) | 4 | (¼x) |
| 29 | 1 | 0.25 | (¼x) | 0.5 | (½x) | 0.5 | (½x) |
| 30 | 0.5 | 0.12 | (¼x) | 0.12 | (¼x) | 0.25 | (½x) |
| 31 | 4 | 0.5 | (⅛x) | 1 | (¼x) | 1 | (¼x) |
| 32 | 2 | 0.25 | (⅛x) | 0.5 | (¼x) | 1 | (½x) |
| 33 | 8 | 2 | (¼x) | 2 | (¼x) | 2 | (¼x) |
| 34 | 8 | 1 | (⅛x) | 1 | (⅛x) | 2 | (¼x) |
| 35 | 4 | 0.5 | (⅛x) | 1 | (¼x) | 1 | (¼x) |
| 36 | 16 | 2 | (⅛x) | 4 | (¼x) | 4 | (¼x) |
| 37 | 4 | 0.5 | (⅛x) | 1 | (¼x) | 1 | (¼x) |
| 38 | 16 | 2 | (⅛x) | 4 | (¼x) | 8 | (½x) |
| 39 | 16 | 1 | ( 1/16x) | 2 | (⅛x) | 4 | (¼x) |
| 40 | 16 | 2 | (⅛x) | 2 | (⅛x) | 4 | (¼x) |
| 41 | 16 | 4 | (¼x) | 4 | (¼x) | 8 | (½x) |
| 42 | 16 | 2 | (⅛x) | 4 | (¼x) | 4 | (¼x) |
| 43 | >16 | 2 | (NA) | 4 | (NA) | 8 | (NA) |
| 44 | >16 | 2 | (NA) | 4 | (NA) | 4 | (NA) |
| 45 | >16 | 16 | (NA) | 16 | (NA) | >16 | (NA) |
| 46 | >16 | 8 | (NA) | 16 | (NA) | 16 | (NA) |
| 47 | 16 | 4 | (¼x) | 4 | (¼x) | 8 | (½x) |
| 48 | >4 | 0.5 | (NA) | 0.5 | (NA) | 1 | (NA) |
| 49 | >16 | 2 | (NA) | 4 | (NA) | 4 | (NA) |
| 50 | 16 | 2 | (⅛x) | 2 | (⅛x) | 4 | (¼x) |
| 51 | 16 | 2 | (⅛x) | 2 | (⅛x) | 4 | (¼x) |
| 52 | >16 | 8 | (NA) | 16 | (NA) | 16 | (NA) |
| 53 | 16 | 2 | (⅛x) | 2 | (⅛x) | 4 | (¼x) |
| 54 | >16 | 2 | (NA) | 4 | (NA) | 8 | (NA) |
| 55 | 0.5 | 0.06 | (⅛x) | 0.12 | (¼x) | 0.12 | (¼x) |
| 56 | 0.5 | 0.12 | (¼x) | 0.25 | (½x) | 0.25 | (½x) |
| 57 | >16 | 8 | (NA) | 16 | (—) | 16 | (NA) |
| 58 | >16 | 4 | (NA) | 4 | (NA) | 4 | (NA) |
| 59 | >16 | 4 | (NA) | 8 | (NA) | 8 | (NA) |
| 60 | 2 | 0.5 | (¼x) | 1 | (½x) | 1 | (½x) |
| 61 | 4 | 0.5 | (⅛x) | 1 | (¼x) | 1 | (¼x) |

TABLE 18
Summary of in vitro Activity of Example Compounds in
Combination with Fluconazole Against C. glabrata: FICI Results.

| | | FICI¹ of combination resulting in fluconazole | | |
|---|---|---|---|---|
| Compound | Methylgene | MIC of: | | |
| Example # | MIC (ug/mL) | 8 ug/mL | 4 ug/mL | 2 ug/mL |
| 1 | 8 | 0.625 (0.188) | 0.500 (0.281) | 0.625 (0.516) |
| 2 | 16 | 0.625 (0.188) | 0.500 (0.281) | 0.375 (0.266) |
| 3 | 0.5 | 0.490 (0.303) | 0.625 (0.531) | 0.563 (0.516) |
| 4 | 0.25 | 0.490 (0.303) | 0.605 (0.511) | 1.063 (1.016) |
| 5 | 4 | 0.280 (0.093) | 0.155 (0.061) | 0.125 (0.078) |
| 6 | 1 | 0.370 (0.183) | 0.245 (0.151) | 0.313 (0.266) |
| 7 | >4 | >0.281 (>0.094) | >0.188 (>0.094) | >0.125 (>0.078) |
| 8 | 0.25 | 0.730 (0.543) | 0.605 (0.511) | 0.543 (0.496) |
| 9 | 0.5 | 0.620 (0.183) | 0.490 (0.271) | 0.625 (0.516) |
| 10 | 2 | 0.625 (0.188) | 0.500 (0.281) | 0.375 (0.266) |
| 11 | 8 | 0.625 (0.188) | 0.375 (0.156) | 0.375 (0.266) |
| 12 | 16 | 0.375 (0.188) | 0.250 (0.156) | 0.313 (0.266) |
| 13 | 4 | 0.375 (0.188) | 0.375 (0.281) | 0.313 (0.266) |
| 14 | >16 | >0.750 (>0.563) | >0.625 (>0.531) | >0.563 (>0.516) |
| 15 | 8 | 0.625 (0.188) | 0.500 (0.281) | 0.375 (0.266) |
| 16 | 2 | 0.625 (0.188) | 0.500 (0.281) | 0.375 (0.266) |
| 19 | 2 | 0.500 (0.313) | 0.375 (0.281) | 0.313 (0.266) |
| 20 | 1 | 0.500 (0.313) | 0.375 (0.281) | 0.563 (0.516) |
| 21 | 16 | 0.375 (0.188) | 0.250 (0.156) | 0.313 (0.266) |
| 22 | 16 | 0.313 (0.125) | 0.250 (0.156) | 0.188 (0.141) |
| 23 | >8 | 0.375 (0.188) | 0.250 (0.156) | 0.313 (0.266) |
| 24 | >16 | 0.313 (0.125) | 0.250 (0.156) | 0.313 (0.266) |
| 25 | >16 | 0.375 (0.188) | 0.375 (0.281) | 0.563 (0.516) |
| 26 | 8 | 0.313 (0.125) | 0.250 (0.156) | 0.313 (0.266) |
| 27 | 16 | 0.375 (0.188) | 0.375 (0.281) | 0.313 (0.266) |
| 28 | 16 | 0.313 (0.125) | 0.250 (0.156) | 0.313 (0.266) |
| 29 | 1 | 0.500 (0.313) | 0.625 (0.531) | 0.563 (0.516) |
| 30 | 0.5 | 0.490 (0.303) | 0.365 (0.271) | 0.563 (0.516) |
| 31 | 4 | 0.625 (0.188) | 0.500 (0.281) | 0.375 (0.266) |
| 32 | 2 | 0.625 (0.188) | 0.500 (0.281) | 0.625 (0.516) |
| 33 | 8 | 0.500 (0.313) | 0.375 (0.281) | 0.313 (0.266) |
| 34 | 8 | 0.625 (0.188) | 0.375 (0.156) | 0.375 (0.266) |
| 35 | 4 | 0.625 (0.188) | 0.500 (0.281) | 0.375 (0.266) |
| 36 | 16 | 0.625 (0.188) | 0.500 (0.281) | 0.375 (0.266) |
| 37 | 4 | 0.625 (0.188) | 0.500 (0.281) | 0.375 (0.266) |
| 38 | 16 | 0.375 (0.188) | 0.375 (0.281) | 0.563 (0.516) |
| 39 | 16 | 0.563 (0.125) | 0.375 (0.156) | 0.375 (0.266) |
| 40 | 16 | 0.375 (0.188) | 0.250 (0.156) | 0.313 (0.266) |
| 41 | 16 | 0.500 (0.313) | 0.375 (0.281) | 0.563 (0.516) |
| 42 | 16 | 0.375 (0.188) | 0.375 (0.281) | 0.313 (0.266) |
| 43 | >16 | 0.313 (0.125) | 0.250 (0.156) | 0.313 (0.266) |
| 44 | >16 | 0.313 (0.125) | 0.250 (0.156) | 0.188 (0.141) |
| 45 | >16 | >0.750 (>0.563) | >0.625 (>0.531) | >1.063 (>1.016) |
| 46 | >16 | 0.500 (0.313) | 0.625 (0.531) | 0.563 (0.516) |
| 47 | 16 | 0.500 (0.313) | 0.375 (0.281) | 0.563 (0.516) |
| 48 | >4 | 0.313 (0.125) | 0.188 (0.094) | 0.188 (0.141) |
| 49 | >16 | 0.313 (0.125) | 0.250 (0.156) | 0.188 (0.141) |
| 50 | 16 | 0.375 (0.188) | 0.250 (0.156) | 0.313 (0.266) |
| 51 | 16 | 0.375 (0.188) | 0.250 (0.156) | 0.313 (0.266) |
| 52 | >16 | >0.500 (>0.313) | >0.625 (>0.531) | >0.563 (>0.516) |
| 53 | 16 | 0.375 (0.188) | 0.250 (0.156) | 0.313 (0.266) |
| 54 | >16 | 0.313 (0.125) | 0.250 (0.156) | 0.313 (0.266) |
| 55 | 0.5 | 0.370 (0.183) | 0.365 (0.271) | 0.303 (0.256) |
| 56 | 0.5 | 0.490 (0.303) | 0.625 (0.531) | 0.563 (0.516) |
| 57 | >16 | 0.500 (0.313) | 0.625 (0.531) | 0.563 (0.516) |
| 58 | >16 | 0.375 (0.188) | 0.250 (0.156) | 0.188 (0.141) |
| 59 | >16 | 0.375 (0.188) | 0.375 (0.281) | 0.313 (0.266) |
| 60 | 2 | 0.500 (0.313) | 0.625 (0.531) | 0.563 (0.516) |
| 61 | 4 | 0.375 (0.188) | 0.375 (0.281) | 0.313 (0.266) |

¹FICI calculated using a fluconazole MIC of 16 μg/mL based on an observed MIC of >8 μg/mL.
Value in parenthesis is the FICI using a fluconazole MIC of 128 μg/mL based on a previously observed MIC of >64 μg/mL.

### REFERENCES

Clinical and Laboratory Standards Institute (CLSI). Reference Method for Broth Dilution Antifungal Susceptibility Testing of Yeasts; Approved Standard—Third Edition. CLSI document M27-A3 [ISBN 1-56238-666-2]. Clinical and Laboratory Standards Institute, 940 West Valley Road, Suite 1400, Wayne, Pa. 19087-1898 USA, 2008.
Clinical and Laboratory Standards Institute (CLSI). Reference Method for Broth Dilution Antifungal Susceptibility Testing of Yeasts; Third Informational Supplement. CLSI document M27-S3 [ISBN 1-56238-667-0]. Clinical and Laboratory Standards Institute, 940 West Valley Road, Suite 1400, Wayne, Pa. 19087-1898 USA, 2008.
Clinical and Laboratory Standards Institute (CLSI). Reference Method for Broth Dilution Antifungal Susceptibility Testing of Filamentous Fungi; Approved Standard—Second Edition. CLSI document M38-A2 [ISBN 1-56238-668-9]. Clinical and Laboratory Standards Institute, 940 West Valley Road, Suite 1400, Wayne, Pa. 19087-1898 USA, 2008.
Eliopoulos G and R Moellering. 1991. Antimicrobial combinations. In Antibiotics in Laboratory Medicine, Third Edition, edited by V Lorian. Williams and Wilkins, Baltimore, Md., pp. 432-492.
Odds FC. 2003. Synergy, antagonism, and what the chequerboard puts between them. J. Antimicrob. Chemother. 52(1):1.

1.-19. (canceled)

20. A histone deacetylase inhibitor of Formula (II): 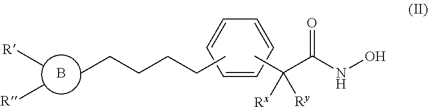
or an N-oxide, hydrate, solvate, pharmaceutically acceptable salt, agricultural formulation, prodrug or complex thereof, wherein
B is aryl, heterocyclic or cycloalkyl;
R′ and R″ are each independently hydrogen, alkoxy, hydroxyl, alkyl, amino, halogen, polyether, —C(O)NR¹R², —O-alkyl-NR¹R², or CH₂C(O)NHOH where
R¹ and R² combine with the nitrogen to which they are attached to form an optionally substituted heterocyclic ring; or
R′ and R″ occur on adjacent carbon atoms and combine to form a fused 1-methyl-2,3-dihydro-1H-pyrrole;
the butyl group is optionally and independently substituted at one or more positions with one or more alkyl, halo or hydroxyl groups, or one oxo, amino or imino group; and
R^{x} and R^{y} are each independently hydrogen or alkyl;
provided that when R^{x} and R^{y} are hydrogen and the butyl group is unsubstituted, B is not 1-H-indole; and
when B is phenyl and the butyl group is unsubstituted, R^{x}, R^{y}, R′ and R″ are not all hydrogen.

21. The inhibitor of claim 20, where B is phenyl.

22. The inhibitor of claim 20, where R¹ and R² are independently methyl, ethyl, isopropyl, 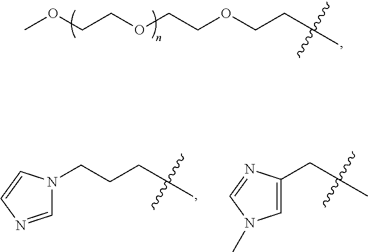
or combine with the nitrogen to which they are attached to form morpholine, pyrrolidine, piperazine, piperidine, 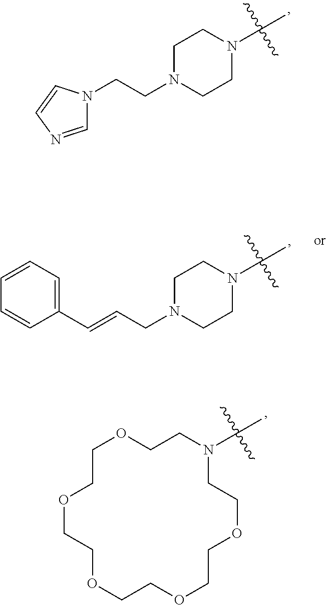
where n is 1-20.

23. The inhibitor of claim 1, where R′ and R″ are independently hydrogen, methoxy, hydroxyl, methyl, fluoro, chloro, bromo, or 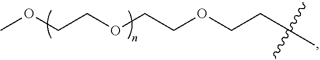
where n is 1-20.

24. The inhibitor of claim 20, where the butyl group is substituted at one or more positions with one or two methyl, ethyl, fluoro, chloro, bromo, or hydroxyl groups, or one oxo, amino, or oxime group.

25. The inhibitor of claim 24, where the butyl group is substituted at one position with one methyl, ethyl, fluoro, chloro, bromo, oxo, amino, oxime or hydroxyl group.

26. The inhibitor of claim 24, where the butyl group is substituted at the same position with two methyl, ethyl, fluoro, chloro, bromo or hydroxyl groups.

27. The inhibitor of claim 20, where the butyl group is unsubstituted.

28. The inhibitor of claim 20, where R^{x} and R^{y} are independently hydrogen or methyl.

29. (canceled)

30. (canceled)

31. The inhibitor of claim 20, wherein the histone deacetylase inhibitor is selected from the group consisting of: 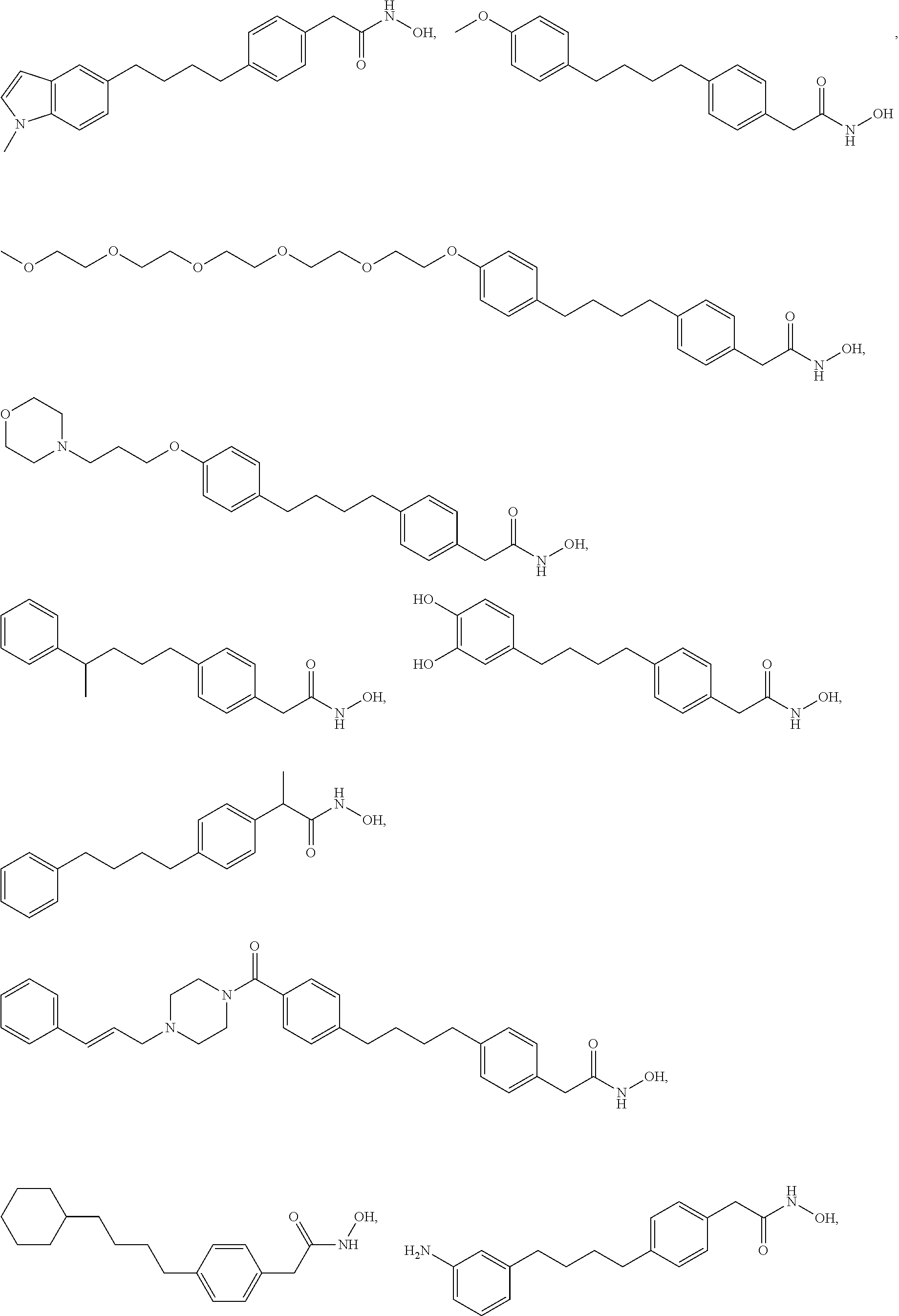 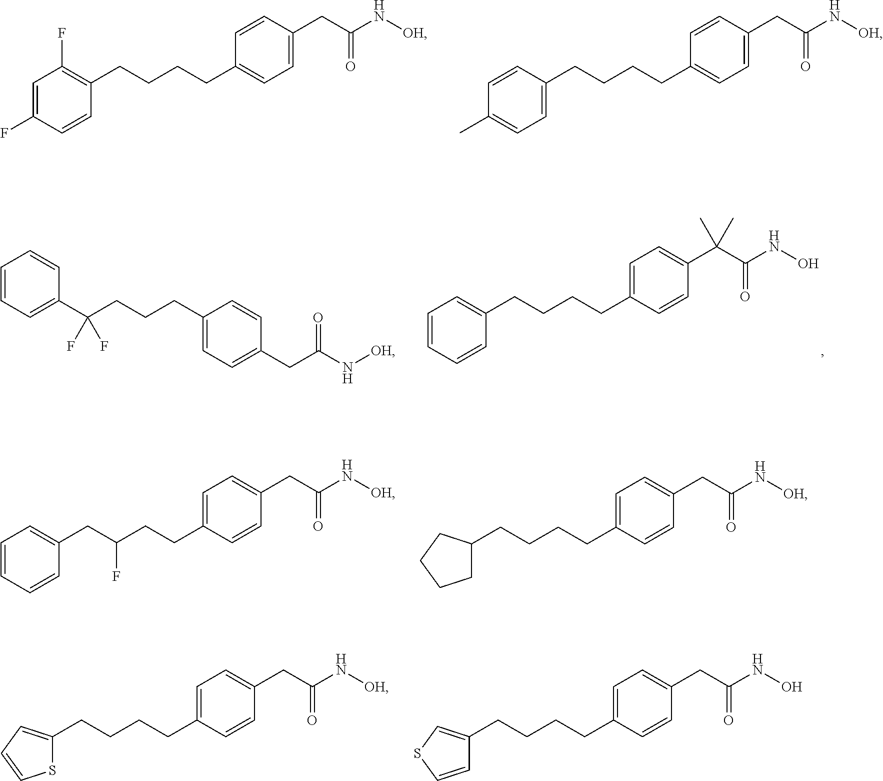
or an N-oxide, hydrate, solvate, pharmaceutically acceptable salt, agricultural formulation, prodrug or complex thereof.

32. (canceled)

33. A histone deacetylase inhibitor of Formula (A-II): 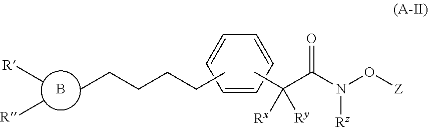
wherein
B is aryl, heterocyclic or cycloalkyl;
R′ and R″ are each independently hydrogen, alkoxy, hydroxyl, alkyl, amino, halogen, polyether, —C(O)NR¹R², —O-alkyl-NR¹R², or CH₂C(O)NHOH where
R¹ and R² combine with the nitrogen to which they are attached to form an optionally substituted heterocyclic ring; or
R′ and R″ occur on adjacent carbon atoms and combine to form a fused 1-methyl-2,3-dihydro-1H-pyrrole;
the butyl group is optionally and independently substituted at one or more positions with one or more alkyl, halo or hydroxyl groups, or one oxo, amino or imino group;
R^{x} and R^{y} are each independently hydrogen or alkyl;
R^{z} is absent and R²⁰ forms an optionally substituted heterocyclic ring with the N to which it is attached;
Z is R²⁰, —OR²⁰, R²¹, —O—C(O)—R¹⁰, —O—C(O)—[C(R¹⁰)(R^{10′})]₁₋₄—NH(R¹³), —OR¹¹ or 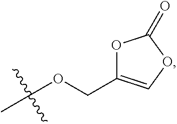
wherein
R²⁰ is selected from the group consisting of —C(O)R¹⁰, —C(O)OR¹⁰, R¹¹, —CH(R¹²)OC(O)R¹⁰, —C(O)[C(R¹⁰)(R^{10′})]₁₋₄—NH(R^{13′}), —S(O₂)R¹⁰, —P(O)(OR¹⁰)(OR¹⁰), —C(O)(CH₂)ₙCH(OH)CH₂OR¹⁰, —C(O)O(CH₂)ₙCH(OH)CH₂OR¹⁰ and —C(O)(CH₂)ₙC(O)OR¹⁰, provided that the N to which Z is bound is not directly bound to two oxygen atoms; or
n is 1-4;
R¹⁰ is selected from the group consisting of hydrogen, optionally substituted C₁-C₂₀ alkyl, optionally substituted C₂-C₂₀ alkenyl, optionally substituted C₂-C₂₀ alkynyl, optionally substituted C₁-C₂₀ alkoxycarbonyl, optionally substituted cycloalkyl, optionally substituted heterocycloalkyl, optionally substituted aryl, optionally substituted heteroaryl, optionally substituted cycloalkylalkyl, optionally substituted heterocycloalkylalkyl, optionally substituted arylalkyl, optionally substituted heteroarylalkyl, optionally substituted cycloalkylalkenyl, optionally substituted heterocycloalkylalkenyl, optionally substituted arylalkenyl, optionally substituted heteroarylalkenyl, optionally substituted cycloalkylalkynyl, optionally substituted heterocycloalkylalkynyl, optionally substituted arylalkynl, optionally substituted heteroarylalkynyl, a sugar residue and an amino acid residue (preferably bonded through the carboxy terminus of the amino acid); or
R^{10′} is hydrogen; or
R¹⁰ and R^{10′} together with the carbon atom to which they are attached form an optionally substituted spirocycloalkyl;
R²¹ is -(amino acid)-R¹³, wherein R¹³ is covalently bound to the N-terminus;
R¹¹ is selected from the group consisting of hydrogen, optionally substituted alkyl, optionally substituted cycloalkyl, optionally substituted heterocycloalkyl, optionally substituted aryl, and optionally substituted heteroaryl;
R¹² is selected from hydrogen or alkyl; and
R¹³ is selected from the group consisting of hydrogen, an amino protecting group and R¹⁰;
with the provisos that when x is 4, n is not 2, and when x is 3, n is not 3.

34. A histone deacetylase inhibitor selected from the group consisting of:
N-hydroxy-2-(2-(4-phenylbutyl)thiazol-4-yl)acetamide,
N-hydroxy-2-(2-(4-phenylbutyl)thiazol-5-yl)acetamide,
2-(4-(4-(2,4-difluorophenyl)butyl)phenyl)-N-hydroxyacetamide,
N-hydroxy-2-(4-(4-p-tolylbutyl)phenyl)acetamide,
2-(4-(4-(biphenyl-4-yl)butyl)phenyl)-N-hydroxyacetamide,
N-hydroxy-2-(4-(4-(1-methyl-1H-indol-5-yl)butyl)phenyl)acetamide,
2,2′-(4,4′-(butane-1,4-diyl)bis(4,1-phenylene))bis(N-hydroxyacetamide),
2-(4-(4-cyclohexylbutyl)phenyl)-N-hydroxyacetamide,
N-hydroxy-2-(4-(4-(4-methoxyphenyl)butyl)phenyl)acetamide,
2-(4-(4-(4-(2,5,8,11,14-pentaoxahexadecan-16-yloxy)phenyl)butyl)phenyl)-N-hydroxyacetamide,
4-(4-(4-(2-(hydroxyamino)-2-oxoethyl)phenyl)butyl)-N-(2-(2-(2-methoxyethoxy)ethoxy)ethyl)benzamide,
N-hydroxy-2-(4-(4-(4-(N-(2-(2-(2-methoxyethoxy)ethoxy)ethyl)sulfamoyl)phenyl)butyl)phenyl)acetamide,
2-(4-(4-(3,4-dimethoxyphenyl)butyl)phenyl)-N-hydroxyacetamide,
4-(4-(4-(2-(hydroxyamino)-2-oxoethyl)phenyl)butyl)benzoic acid,
N-hydroxy-2-(4-(4-(4-hydroxyphenyl)butyl)phenyl)acetamide,
N-hydroxy-2-(4-(4-(4-(3-morpholinopropoxy)phenyl)butyl)phenyl)acetamide,
2-(4-(4-(4-(3-(dimethylamino)propoxy)phenyl)butyl)phenyl)-N-hydroxyacetamide,
N-hydroxy-2-(4-(4-(4-(2-(pyrrolidin-1-yl)ethoxy)phenyl)butyl)phenyl)acetamide,
2-(4-(4-(3,4-dihydroxyphenyl)butyl)phenyl)-N-hydroxyacetamide,
(E)-2-(4-(4-(4-(4-cinnamylpiperazine-1-carbonyl)phenyl)butyl)phenyl)-N-hydroxyacetamide,
2-(4-(4-(4-(4-(2-(1H-imidazol-1-yl)ethyl)piperazine-1-carbonyl)phenyl)butyl)phenyl)-N-hydroxyacetamide,
N-(3-(1H-imidazol-1-yl)propyl)-4-(4-(4-(2-(hydroxyamino)-2-oxoethyl)phenyl)butyl)benzamide,
4-(4-(4-(2-(hydroxyamino)-2-oxoethyl)phenyl)butyl)-N-((1-methyl-1H-imidazol-4-yl)methyl)benzamide,
2-(4-(4-(4-(1,4,7,10,13-pentaoxa-16-azacyclooctadecane-16-carbonyl)phenyl)butyl)phenyl)-N-hydroxyacetamide,
N-hydroxy-2-(4-(4-hydroxy-4-phenylbutyl)phenyl)acetamide,
2-(4-(4-fluoro-4-phenylbutyl)phenyl)-N-hydroxyacetamide,
(E)-N-hydroxy-2-(4-(4-(hydroxyimino)-4-phenylbutyl)phenyl)acetamide,
N-hydroxy-2-(4-(4-oxo-4-phenylbutyl)phenyl)acetamide,
2-(4-(4,4-difluoro-4-phenylbutyl)phenyl)-N-hydroxyacetamide,
N-hydroxy-2-(4-(4-phenylpentyl)phenyl)acetamide,
2-(4-(4-(4-aminophenyl)butyl)phenyl)-N-hydroxyacetamide,
2-(4-(4-(3-aminophenyl)butyl)phenyl)-N-hydroxyacetamide,
2-(4-(4-(2-aminophenyl)butyl)phenyl)-N-hydroxyacetamide,
N-(4-(4-(4-(2-(hydroxyamino)-2-oxoethyl)phenyl)butyl)phenyl)-1-methylpiperidine-4-carboxamide hydrochloride,
N-hydroxy-2-(4-(4-(4-(2-(2-(2-methoxyethoxy)ethoxy)acetamido)phenyl)butyl)phenyl)acetamide,
N-hydroxy-2-(4-(4-(4-(2-hydroxyacetamido)phenyl)butyl)phenyl)acetamide,
N-(4-(4-(4-(2-(hydroxyamino)-2-oxoethyl)phenyl)butyl)phenyl)-2,5,8,11-tetraoxatetradecan-14-amide,
2-(2-(dimethylamino)ethylthio)-N-(4-(4-(4-(2-(hydroxyamino)-2-oxoethyl)phenyl)butyl)phenyl)acetamide,
2-(4-(4-(4-acetamidophenyl)butyl)phenyl)-N-hydroxyacetamide,
N-hydroxy-2-(4-(4-(3-(2-(2-(2-methoxyethoxy)ethoxy)acetamido)phenyl)butyl)phenyl)acetamide,
N-hydroxy-2-(4-(4-(3-(2-hydroxyacetamido)phenyl)butyl)phenyl)acetamide,
2-(4-(4-(3-acetamidophenyl)butyl)phenyl)-N-hydroxyacetamide,
N-(3-(4-(4-(2-(hydroxyamino)-2-oxoethyl)phenyl)butyl)phenyl)-1-methylpiperidine-4-carboxamide,
2-(2-(dimethylamino)ethylthio)-N-(3-(4-(4-(2-(hydroxyamino)-2-oxoethyl)phenyl)butyl)phenyl)acetamide,
2-(4-(4-(2-acetamidophenyl)butyl)phenyl)-N-hydroxyacetamide,
N-hydroxy-2-(4-(4-(2-(2-hydroxyacetamido)phenyl)butyl)phenyl)acetamide,
N-hydroxy-2-(4-(4-(4-(3-(2-morpholinoethyl)ureido)phenyl)butyl)phenyl)acetamide,
methyl 4-(4-(4-(2-(hydroxyamino)-2-oxoethyl)phenyl)butyl)phenylcarbamate,
N-hydroxy-2-(4-(4-(3-(3-(2-(4-methylpiperazin-1-yl)ethyl)ureido)phenyl)butyl)phenyl)acetamide,
N-hydroxy-2-(4-(4-(3-(3-(2-morpholinoethyl)ureido)phenyl)butyl)phenyl)acetamide,
N-hydroxy-2-(4-(4-(4-(2-morpholinoethylsulfonamido)phenyl)butyl)phenyl)acetamide,
N-hydroxy-2-(4-(4-(4-(2-(4-methylpiperazin-1-yl)ethylsulfonamido)phenyl)butyl)phenyl)acetamide,
N-hydroxy-2-(4-(4-(3-(2-morpholinoethylsulfonamido)phenyl)butyl)phenyl)acetamide,
N-hydroxy-2-(4-(4-(3-(2-(4-methylpiperazin-1-yl)ethylsulfonamido)phenyl)butyl)phenyl)acetamide,
N-hydroxy-2-(4-(4-phenylbutyl)phenyl)propanamide,
N-hydroxy-2-methyl-2-(4-(4-phenylbutyl)phenyl)propanamide,
N-hydroxy-2-(4-(3-hydroxy-4-phenylbutyl)phenyl)acetamide,
N-hydroxy-2-(4-(3-hydroxy-4-phenylbutyl)phenyl)propanamide,
N-hydroxy-2-(4-(1-hydroxy-4-phenylbutyl)phenyl)acetamide,
2-(4-(3-fluoro-4-phenylbutyl)phenyl)-N-hydroxyacetamide,
2-(4-(1-fluoro-4-phenylbutyl)phenyl)-N-hydroxyacetamide,
N-hydroxy-2-(5-(4-phenylbutyl)furan-2-yl)acetamide,
2-(4-(4-Cyclopentylbutyl)phenyl)-N-hydroxyacetamide,
N-Hydroxy-2-(4-(4-(thiophen-2-yl)butyl)phenyl)acetamide,
N-Hydroxy-2-(4-(4-(thiophen-3-yl)butyl)phenyl)acetamide,
and an N-oxide, hydrate, solvate, pharmaceutically acceptable salt, agricultural formulation, prodrug or complex thereof.

35. The inhibitor of claim 20, wherein the histone deacetylase inhibitor is selected from the group consisting of:
2-(4-(4-(2,4-difluorophenyl)butyl)phenyl)-N-hydroxyacetamide,
N-hydroxy-2-(4-(4-p-tolylbutyl)phenyl)acetamide,
N-hydroxy-2-(4-(4-(1-methyl-1H-indol-5-yl)butyl)phenyl)acetamide,
2-(4-(4-cyclohexylbutyl)phenyl)-N-hydroxyacetamide,
N-hydroxy-2-(4-(4-(4-methoxyphenyl)butyl)phenyl)acetamide,
2-(4-(4-(4-(2,5,8,11,14-pentaoxahexadecan-16-yloxy)phenyl)butyl)phenyl)-N-hydroxyacetamide,
N-hydroxy-2-(4-(4-(4-(3-morpholinopropoxy)phenyl)butyl)phenyl)acetamide,
2-(4-(4-(3,4-dihydroxyphenyl)butyl)phenyl)-N-hydroxyacetamide,
(E)-2-(4-(4-(4-(4-cinnamylpiperazine-1-carbonyl)phenyl)butyl)phenyl)-N-hydroxyacetamide,
2-(4-(4,4-difluoro-4-phenylbutyl)phenyl)-N-hydroxyacetamide,
N-hydroxy-2-(4-(4-phenylpentyl)phenyl)acetamide,
2-(4-(4-(3-aminophenyl)butyl)phenyl)-N-hydroxyacetamide,
N-hydroxy-2-(4-(4-phenylbutyl)phenyl)propanamide,
N-hydroxy-2-methyl-2-(4-(4-phenylbutyl)phenyl)propanamide,
2-(4-(3-fluoro-4-phenylbutyl)phenyl)-N-hydroxyacetamide,
2-(4-(4-Cyclopentylbutyl)phenyl)-N-hydroxyacetamide,
N-Hydroxy-2-(4-(4-(thiophen-2-yl)butyl)phenyl)acetamide,
N-Hydroxy-2-(4-(4-(thiophen-3-yl)butyl)phenyl)acetamide,
and an N-oxide, hydrate, solvate, pharmaceutically acceptable salt, agricultural formulation, prodrug or complex thereof.

36.-60. (canceled)
